(19) United States
(12) Patent Application Publication
(10) Pub. No.: US 2024/0207224 A1
(43) Pub. Date: Jun. 27, 2024

(54) MACROCYCLIC CHALCONE-AMIDE DERIVED ANTIVIRAL AGENTS

(71) Applicant: ENANTA PHARMACEUTICALS, INC., WATERTOWN, MA (US)

(72) Inventors: Jiajun Zhang, Cambridge, MA (US); Xuri Gao, Newtonville, MA (US); Hui Cao, Belmont, MA (US); Yuk Ming Siu, Watertown, MA (US); Bin Wang, Newton, MA (US); Jiang Long, Wayland, MA (US); Wei Li, Lexington, MA (US); Matthew C. Rhodes, Boston, MA (US); Xuechao Xing, Wilmington, MA (US); Scott Mitchell, Woburn, MA (US); Yat Sun Or, Waltham, MA (US)

(21) Appl. No.: 18/509,403

(22) Filed: Nov. 15, 2023

Related U.S. Application Data
(60) Provisional application No. 63/425,421, filed on Nov. 15, 2022.

(51) Int. Cl. A61K 31/395; A61K 31/4192; A61K 31/4995; A61P 31/14; C07D 225/04; C07D 245/04; C07D 255/04; C07D 273/02; C07D 281/00; C07D 285/00; C07D 487/08

(52) U.S. Cl. CPC A61K 31/395; A61K 31/4192; A61K 31/4995; A61P 31/14; C07D 225/04; C07D 245/04; C07D 255/04; C07D 273/02; C07D 281/00; C07D 285/00; C07D 487/08

(57) ABSTRACT

The present invention discloses compounds of Formula (I), and pharmaceutically acceptable salts, thereof:

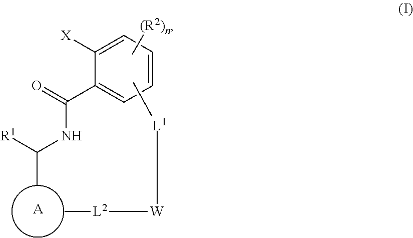

which inhibit coronavirus replication activity. The invention further relates to pharmaceutical compositions comprising a compound of Formula (I) or a pharmaceutically acceptable salt thereof, and methods of treating or preventing a coronavirus infection in a subject in need thereof, comprising administering to the subject a therapeutically effective amount of a compound of Formula (I) or a pharmaceutically acceptable salt thereof.

### RELATED APPLICATION

This application claims the benefit of U.S. Provisional Application No. 63/425,421, filed on Nov. 15, 2022. The entire teachings of the above application are incorporated herein by reference.

### TECHNICAL FIELD

The invention relates to compounds and methods for treating or preventing a coronavirus infection. The invention further relates to pharmaceutical compositions comprising a compound of the invention.

### BACKGROUND OF THE INVENTION

Coronaviruses are enveloped, positive-sense, single-stranded RNA viruses. The genomic RNA of CoVs has a 5′-cap structure and 3′-poly-A tail and contains at least 6 open reading frames (ORFs). The first ORF (ORF 1a/b) directly translates two polyproteins: pp1a and pp1ab.

These polyproteins are processed by two essential proteases 3C-Like protease (3CLpro), also known as the main protease (Mpro), and Papain-Like protease (PLpro) into 16 non-structural proteins. These non-structural proteins engage in the production of subgenomic RNAs that encode four structural proteins, namely envelope, membrane, spike, and nucleocapsid proteins, among other accessory proteins. As a result, it is understood that both 3CLpro and PLpro have critical roles in the coronavirus life cycle.

In addition, PLpro is involved in antagonizing the host's immune response upon viral infection. PLpro has deubiquitinating and deISGylating activities and removes ubiquitin and ISG15 modifications from host proteins, leading to suppression of the innate immune response and promotion of viral replication. The deubiquitinating and deISGylating activities of PLpro are indispensable in antagonizing the host's immune response. Recent studies showed that SARS-CoV-2 infection of human macrophages triggers the release of extracellular free ISG15 through the viral PLpro, leading to the subsequent secretion of proinflammatory cytokines and chemokines, which recapitulates the cytokine storm of COVID-19. This finding suggests that inhibiting the PLpro activity might alleviate the hyper-inflammation in COVID patients. Thus, targeting PLpro is expected to not only suppress viral replication but also restore antiviral immunity in the host.

There are two types of PLpros: PL1pro and PL2pro. They have distinct substrate specificities in different coronaviruses. The coronaviruses HCoV-220E, HCoV-NL63, HCoV-HKU1, and HCoV-OC43 encode both PL1pro and PL2pro. In contrast, SARS-CoV, MERS-CoV, and SARS-CoV-2 comprise only one functional PL2pro. The SARS-CoV-2 PLpro is part of nsp3, a 215-kDa multidomain viral protein. It specifically recognizes a consensus cleavage motif, LXGG↓(N/L/X), which is present in between nsp1/2, nsp2/3, and nsp3/4 at the viral polyprotein as well as the C-terminal sequences of ubiquitin and ISG15 with an isopeptide bond.

PLpro is a cysteine protease, containing four domains: the thumb, palm, zinc-finger domain, and an N-terminal ubiquitin-like domain. The catalytic triad consists of Cys, His and Asp, which are located at the interface of the palm and thumb domains. The zinc-finger motif comprises four cysteines coordinating with a zinc ion and is vital for the structural integrity and protease activity of PLpro. The flexible BL2 loop undergoes conformational changes from open to closed upon substrate binding. This site is also the drug binding site for GRL-0617 and its analogs which have been reported to inhibit PLpro activity (refer to WO2021189046, WO2022070048, WO2022072975, WO2022169891, WO2022189810, WO2022192665). However, no PLpro inhibitor has been advanced to the clinic.

Although Paxlovid (3CLpro inhibitor) and Lagevrio (RdRp inhibitor) have been approved as the first-generation oral antiviral therapies via EUA, more effective oral therapies with different MOAs for coronavirus infections are needed because the new therapies could overcome the potential drug resistance of current therapies. This invention describes the methods to prepare and methods for use of compounds that are believed to inhibit the coronavirus lifecycle. Compounds of this type might be used to treat coronavirus infections and decrease the occurrence of disease complications such as organ failure or death.

There is a need in the art for novel therapeutic agents that treat, ameliorate or prevent coronavirus infection. Administration of these therapeutic agents to a coronavirus infected patient, either as monotherapy or in combination with other coronavirus treatments or ancillary treatments, will lead to significantly improved prognosis, diminished progression of the disease, and enhanced seroconversion rates.

### SUMMARY OF THE INVENTION

The present invention relates to novel antiviral compounds, pharmaceutical compositions comprising such compounds, and methods for treating or preventing a viral (particularly coronavirus) infection in a subject in need of such therapy with said compounds. In addition, the present invention provides processes for the preparation of said compounds.

Compounds of the present invention inhibit the coronavirus Papain-Like protease (PLpro), thus interfering with the life cycle of the coronavirus and restoring host antiviral immunity.

The present invention provides compounds represented by Formula (I), and pharmaceutically acceptable salts, esters and prodrugs thereof,

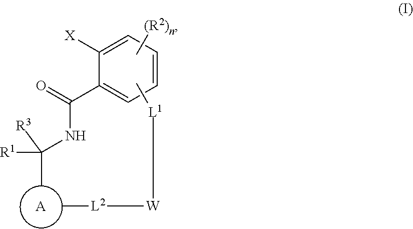

wherein:
A is selected from the group consisting of optionally substituted aryl, optionally substituted heteroaryl, optionally substituted —C₃-C₈ cycloalkyl, and optionally substituted 3- to 8-membered heterocycloalkyl;
R¹ and R³ are each independently selected from the group consisting of hydrogen, optionally substituted —C₁-C₄ alkyl, optionally substituted —C₂-C₄ alkenyl, optionally substituted —C₂-C₄ alkynyl, and optionally substituted —C₃-C₆ cycloalkyl; preferably R¹ is optionally substituted methyl; more preferably R¹ is methyl;
alternatively, R¹ and R³ are taken together with the carbon atom to which they are attached to form an optionally substituted 3- to 8-membered carbocyclic or 3- to 8-membered heterocyclic ring;
R² is selected from the group consisting of halogen, —OR¹¹, —OC(O)R¹¹, —C(O)OR¹¹, —OC(O)NR¹²R¹³, —NR¹²R¹³, —NR¹²C(O)R¹¹, —NR¹²C(O)OR¹³, —NR¹²C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
each R¹¹ is independently selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
R¹² and R¹³ at each occurrence are independently selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; alternatively R¹² and R¹³ are taken together with the nitrogen atom to which they are attached to form an optionally substituted 3- to 8-membered heterocyclic ring;
n is 0, 1, 2, or 3;
L¹ and L² are independently selected from the group consisting of —CR²¹═CR²²—, —CR²¹R²³—CR²²R²⁴—, —(CR²¹R²³)ₘ—O—, —(CR²¹R²³)ₘ—S—, —(CR²¹R²³)ₘ—NR¹²—,

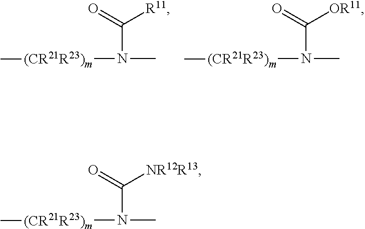

—(CR²¹R²³)ₘ—NR¹²C(O)—, —(CR²¹R²³)ₘ—NR¹²C(O)O—, —(CR²¹R²³)ₘ—NR¹²C(O)NR¹³—, —(CR²¹R²³)ₘ—C(O)N(R¹²)—, —(CR²¹R²³)ₘ—C(O)—, —(CR²¹R²³)ₘ—S(O)₂—, —(CR²¹R²³)ₘ—S(O)(NH)—, —(CR²¹R²³)ₘ—S(O)₂NR¹²—, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
m is 0, 1, 2, 3 or 4.

R²¹ and R²² at each occurrence are independently selected from the group consisting of hydrogen, halogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; in certain embodiments, R²¹ and R²² are both hydrogen;
R²³ and R²⁴ at each occurrence are independently selected from the group consisting of hydrogen, halogen, —OR¹¹, —OC(O)R¹¹, —OC(O)OR¹¹, —OC(O)NR¹²R¹³, —NR¹²R¹³, —NR¹²C(O)R¹¹, —NR¹²C(O)OR¹¹, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; in certain embodiments, R²³ and R²⁴ are both hydrogen;
W is selected from the group consisting of optionally substituted —C₁-C₈ alkyl, optionally substituted —C₃-C₈ cycloalkyl, optionally substituted 3- to 8-membered heterocycloalkyl, optionally substituted aryl, optionally substituted arylalkyl, optionally substituted heteroaryl, optionally substituted heteroarylalkyl; —(CR²¹R²³)_{q1}—O—(CR²²R²⁴)_{q2}—, —(CR²¹R²³)_{q1}—NR¹²—(CR²²R²⁴)_{q2}—, and —(CR²¹R²³)_{q1}—S—(CR²²R²⁴)_{q2}—;
q1 is 0, 1, 2, 3, 4, 5, 6, 7, or 8; preferably q1 is 0, 1, 2, 3 or 4;
q2 is 0, 1, 2, 3, 4, 5, 6, 7, or 8; preferably q1 is 0, 1, 2, 3 or 4;
X is selected from the group consisting of halogen, optionally substituted —C₁-C₆ alkyl, optionally substituted —C₂-C₆ alkenyl, optionally substituted —C₃-C₆ cycloalkyl,

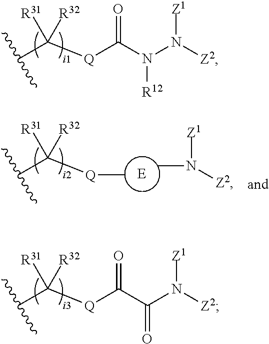

Z¹ and Z² are each independently an amino protecting group, R¹³,

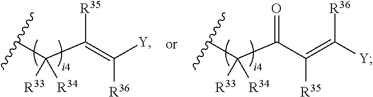

R³¹, R³², R³³ and R³⁴ at each occurrence are independently selected from the group consisting of hydrogen, optionally substituted —CH₃, and halogen; in certain embodiments, each R³¹, R³², R³³ and R³⁴ is hydrogen;
R³⁵ and R³⁶ are each independently selected from the group consisting of hydrogen, optionally substituted —CH₃, halogen, and —CN; in certain embodiments, R³⁵ and R³⁶ are each hydrogen;
i1, i2, i3, or i4 are each independently is 0, 1, 2, 3, or 4, preferably 0, 1, or 2.
Q is selected from absent, —O—, and —NR¹²—.
E is selected from optionally substituted aryl, optionally substituted heteroaryl; and
Y is selected from the group consisting of hydrogen, halogen, —CF₃; —CN, —C(O)R¹¹, —C(O)OR¹², —C(O)NR¹²R¹³, —S(O)₂R¹¹, —S(O)₂NR¹²R¹³, optionally substituted aryl, and optionally substituted heteroaryl.

### DETAILED DESCRIPTION OF THE INVENTION

In one embodiment of the present invention is a compound of Formula (I) as described above, or a pharmaceutically acceptable salt thereof.

In certain embodiments of the compounds of Formula (I), R¹ is optionally substituted —C₁-C₄ alkyl or optionally substituted —C₃-C₅ cycloalkyl.

In certain embodiments of the compounds of Formula (I), R¹ is methyl or —CD₃.

In certain embodiments of the compounds of Formula (I), R³ is hydrogen.

In certain embodiments of the compounds of Formula (I), R¹ is methyl or —CD₃, and R³ is hydrogen.

In certain embodiments of the compounds of Formula (I), R¹ and R³ are taken together with the carbon atom to which they are attached to form an optionally substituted cyclopropyl.

In certain embodiments of the compounds of Formula (I), n is 1 or 2 and each R² is halogen.

In certain embodiments of the compounds of Formula (I), n is 0.

In certain embodiments of the compounds of Formula (I), X is halogen, methyl, —CD₃, ethyl, cyclopropyl, —CH₂F, —CHF₂, —CF₃, or —CH═CH₂.

In certain embodiments of the compounds of Formula (I), X is

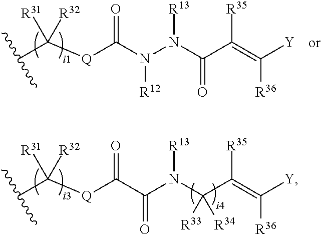

wherein Q, Y, i1, i3, i4, R¹², R¹³, R³¹, R³², R³³, R³⁴, R³⁵, and R³⁶ are as previously defined.

In certain embodiments of the compounds of Formula (I), X is

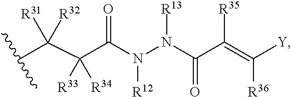

wherein Y, R³¹, R³², R³³, R³⁴, R³⁵ and R³⁶ are as previously defined.

In certain embodiments of the compounds of Formula (I), X is

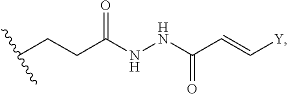

wherein Y is as previously defined.

In certain embodiments of the compounds of Formula (I), X is

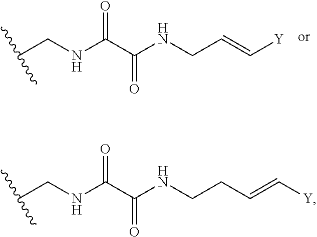

wherein Y is as previously defined.

In certain embodiments of the compounds of Formula (I), X is methyl, —CD₃,

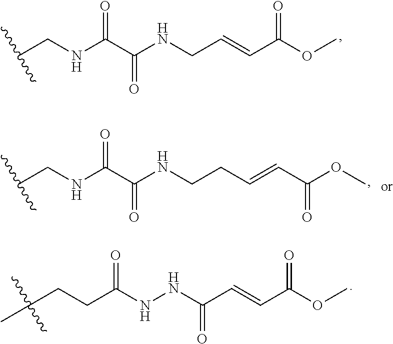

In certain embodiments of the compounds of Formula (I), A is optionally substituted aryl or optionally substituted heteroaryl.

In certain embodiments of the compounds of Formula (I), A is derived from one of the following by removal of two hydrogen atoms and is optionally substituted:

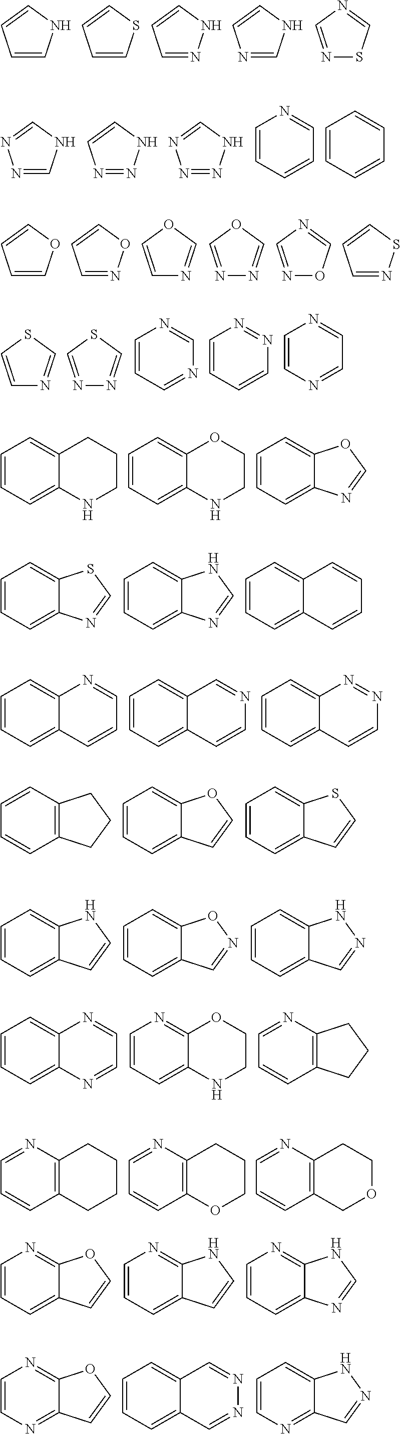

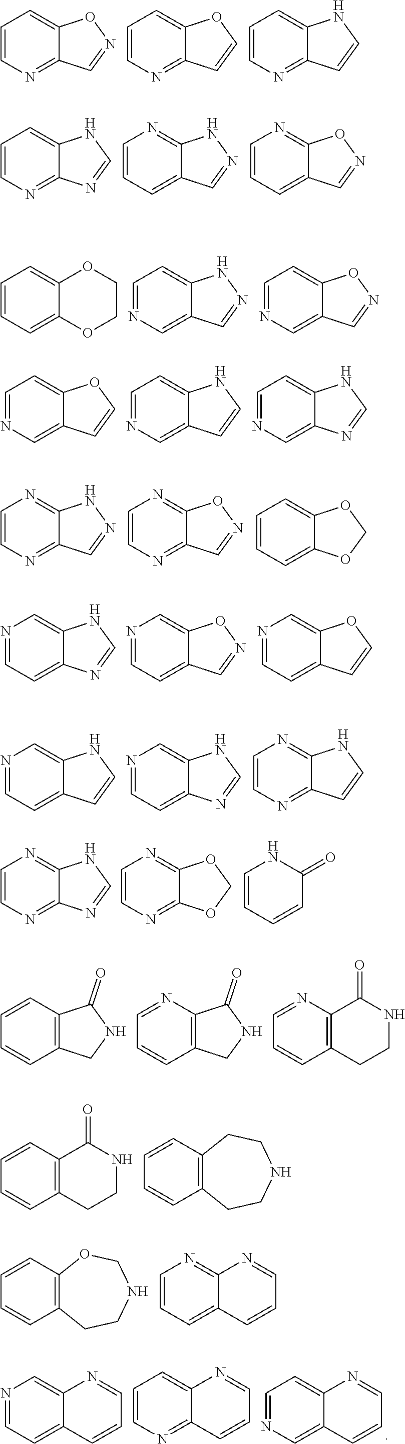

In certain embodiments of the compounds of Formula (I), A is optionally substituted phenyl or optionally substituted naphthyl.

In certain embodiments of the compounds of Formula (I), L¹ is —CH₂—CH₂—, —CH═CH—, —C(O)NR¹²—, —CH₂—NR¹²—, —CH₂—NR¹²C(O)—, or —O—CH₂—. In one embodiment L¹ connects to phenyl and W in Formula (I) independently through carbon, nitrogen, or oxygen atom.

In certain embodiments of the compounds of Formula (I), L² is —CH₂—CH₂—, —CH═CH—, —C(O)NR¹²—, —O—CH₂—, —NR¹²—CH₂—, optionally substituted heteroaryl, or optionally substituted heterocycloalkyl. In one embodiment L² connects to A and W in Formula (I) independently through carbon, nitrogen, or oxygen atom.

In certain embodiments of the compounds of Formula (I), W is —(CH₂)ₘ—, wherein m is 3, 4, or 5.

In certain embodiments, the compound of Formula (I) is represented by Formula (I-a):

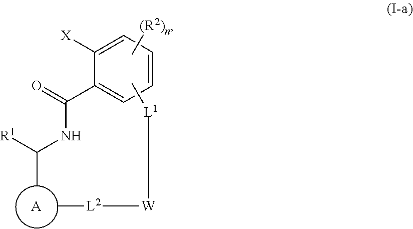

wherein A, X, R¹, R², L¹, L², W and n are as previously defined.

In certain embodiments, the compound of Formula (I) is represented by Formula (I-a′):

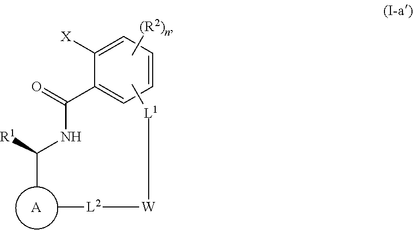

wherein A, X, R¹, R², L¹, L², W and n are as previously defined.

In certain embodiments, the compound of Formula (I) is represented by Formula (II):

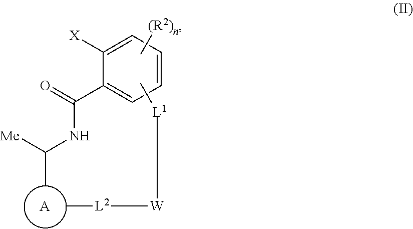

wherein A, X, R², L¹, L², W and n are as previously defined.

In certain embodiments, the compound of Formula (I) is represented by one of Formulae (III-1)˜(III-4):

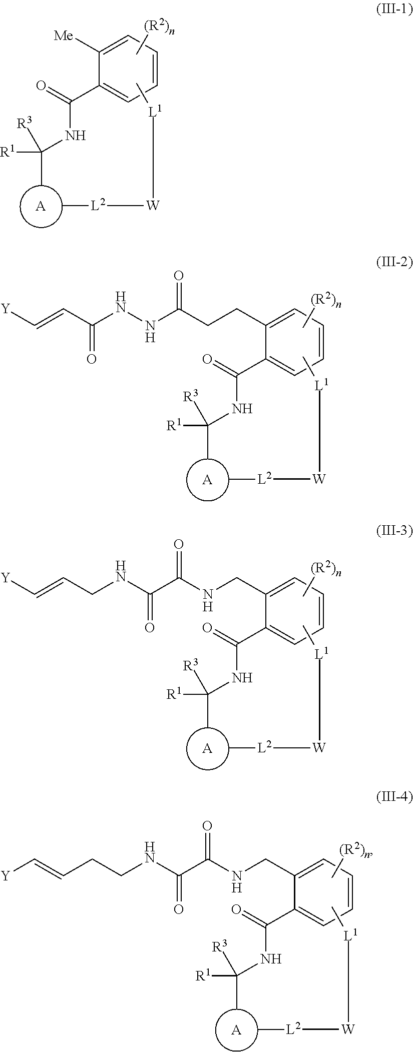

wherein A, R¹, R², R³, L¹, L², W, n and Y are as previously defined.

In certain embodiments, the compound of Formula (I) is represented by one of Formulae (III-1a)˜(III-4a):

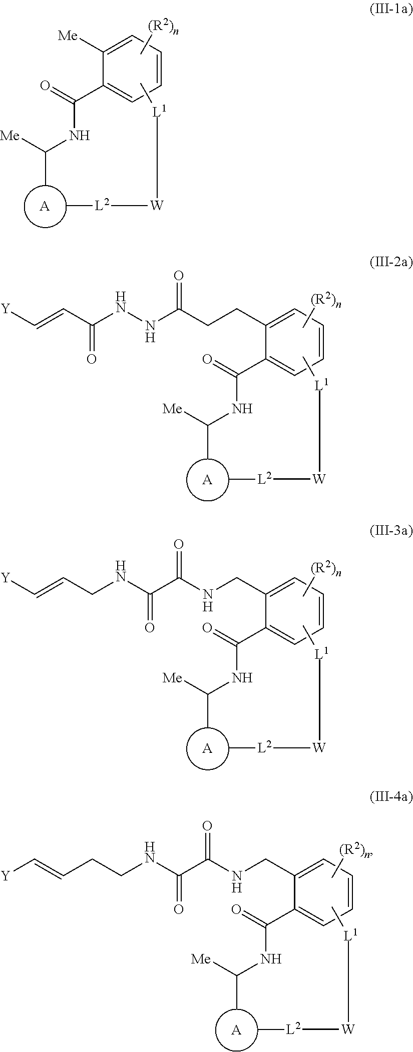

wherein A, R², L¹, L², W, n and Y are as previously defined.

In certain embodiments, the compound of Formula (I) is represented by one of Formulae (IV-1)˜(IV-8):

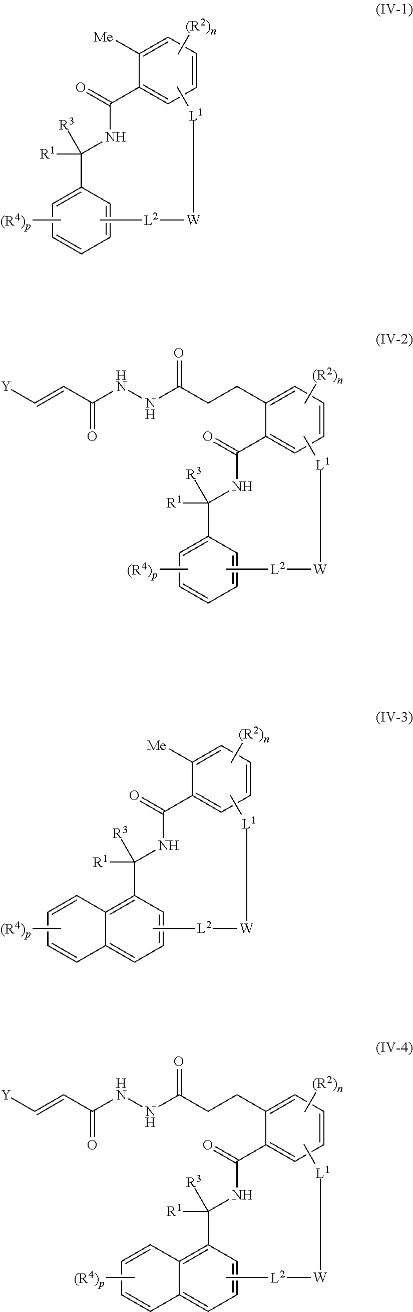

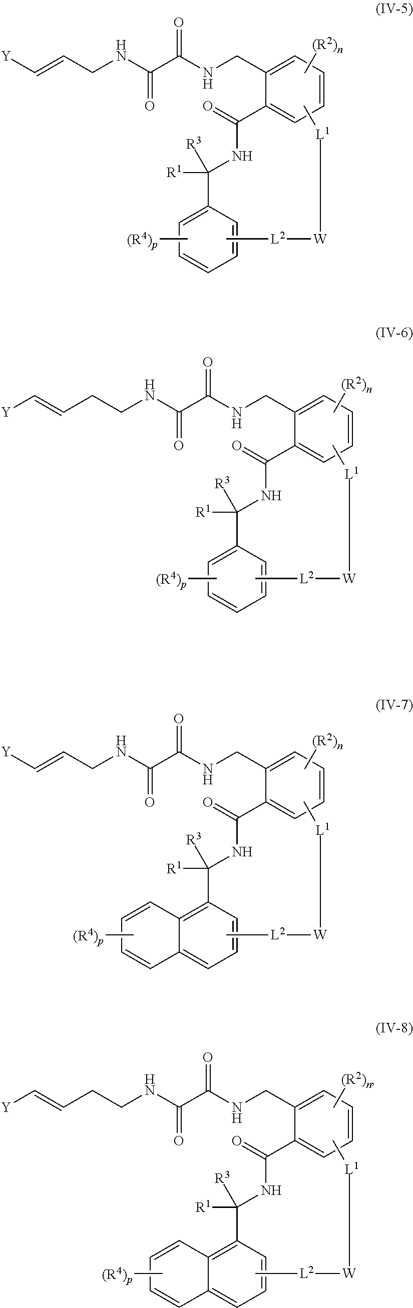

wherein R⁴ is selected from the group consisting of halogen, —OR¹¹, —OC(O)R¹¹, —C(O)OR¹¹, —OC(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; p is 0, 1, 2, 3, or 4; and R¹, R², R³, R¹¹, R¹², R¹³, L¹, L², W, and n are as previously defined.

In certain embodiments, the compound of Formula (I) is represented by one of Formulae (IV-1a)˜(IV-8a):

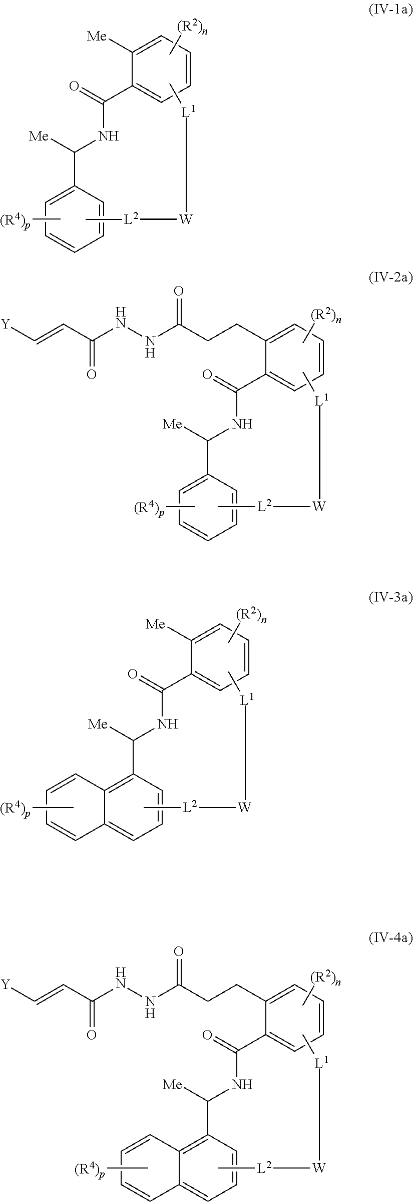

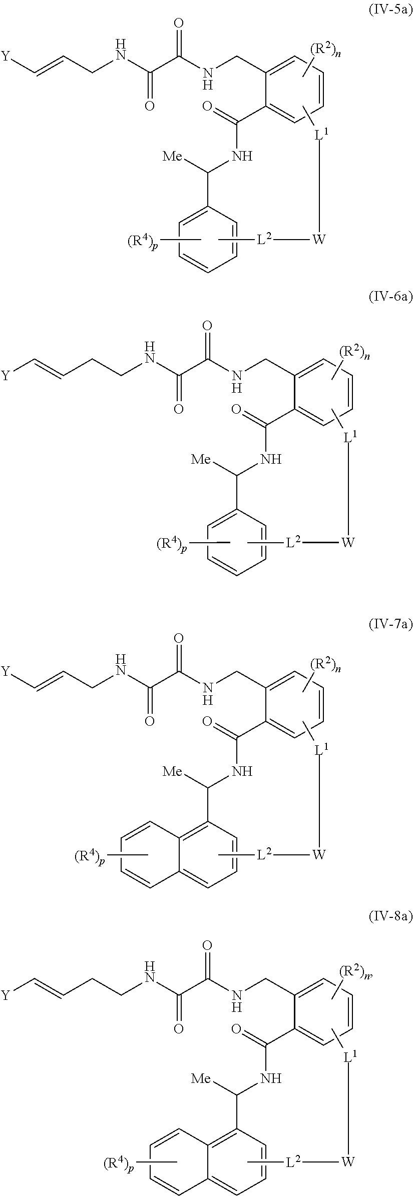

wherein R², R⁴, R¹¹, R¹², R¹³, L¹, L², W, n, p, and Y are as previously defined.

In certain embodiments, the compound of Formula (I) is represented by one of Formulae (V-1)˜(V-8):

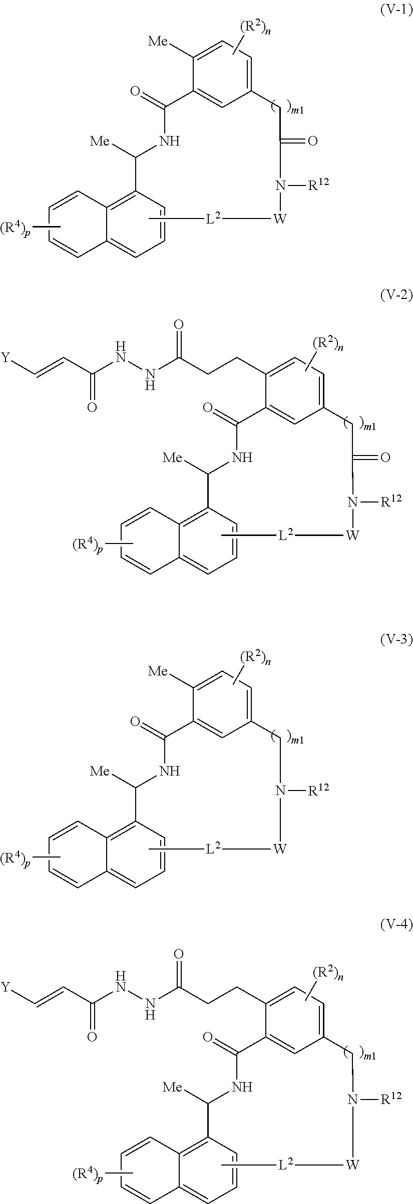

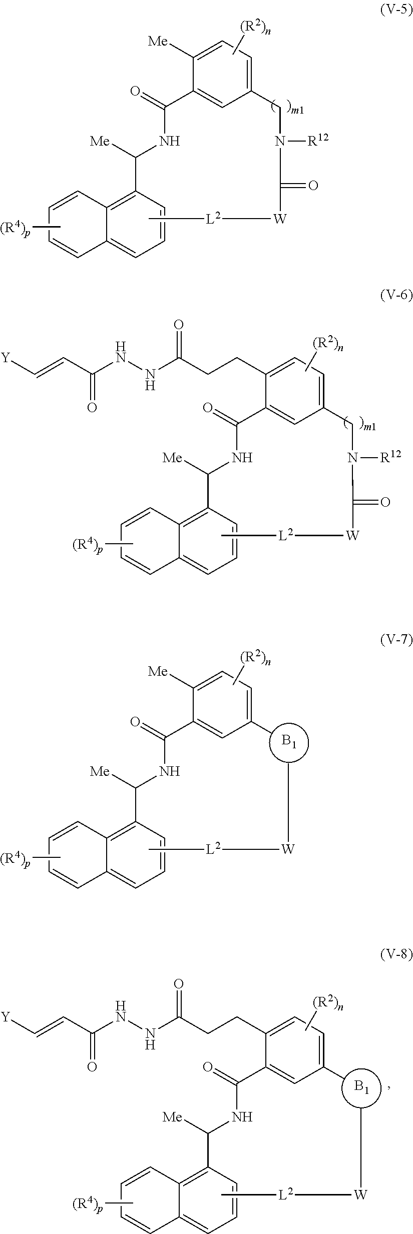

wherein B₁ is selected from the group consisting of optionally substituted aryl, optionally substituted heteroaryl, optionally substituted —C₃-C₈ cycloalkyl, and optionally substituted 3- to 8-membered heterocycloalkyl; m1 is 0, 1 or 2; and R², R⁴, R¹², L², W, n, p, and Y are as previously defined.

In certain embodiments, the compound of Formula (I) is represented by one of Formulae (VI-1)˜(VI-6):

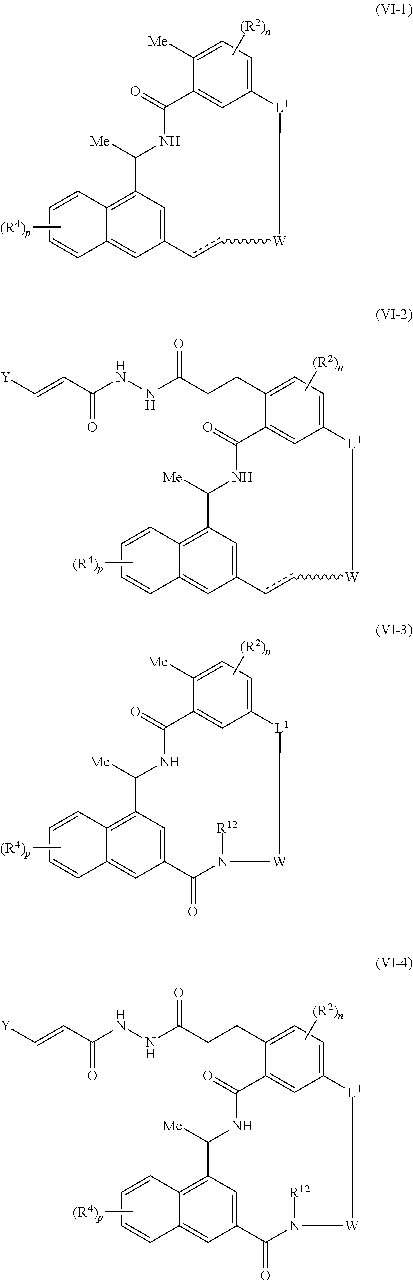

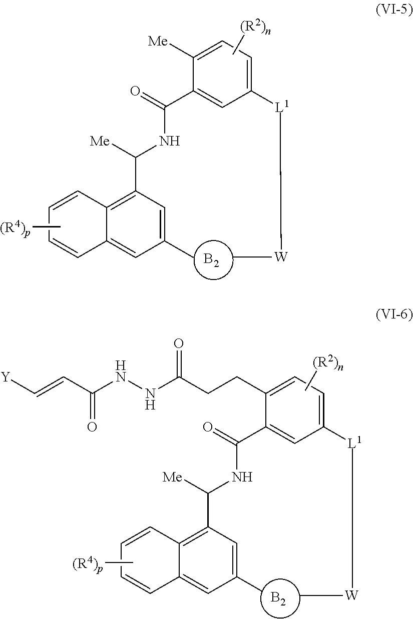

wherein B₂ is selected from the group consisting of optionally substituted aryl, optionally substituted heteroaryl, optionally substituted —C₃-C₈ cycloalkyl, and optionally substituted 3- to 8-membered heterocycloalkyl; and R², R⁴, R¹², L¹, W, n, p, and Y are as previously defined.

In certain embodiments, the compound of Formula (I) is represented by one of Formulae (VII-1)˜(VII-6):

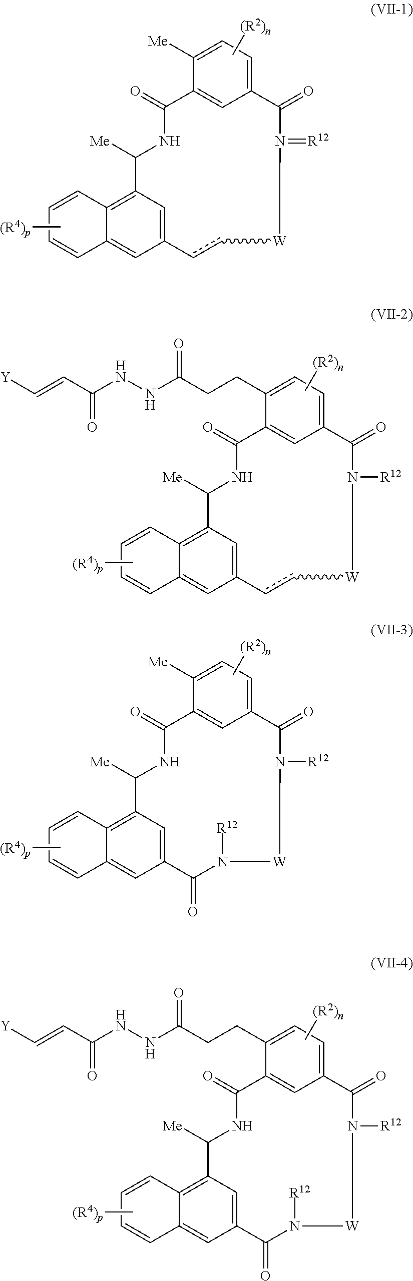

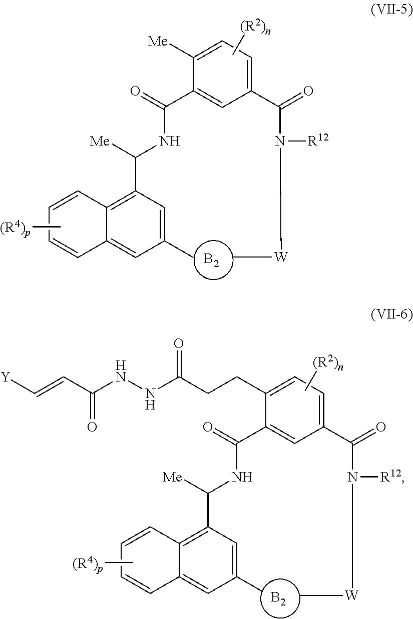

wherein B₂, R², R⁴, R¹², W, n, p, and Y are as previously defined.

In certain embodiments, the compound of Formula (I) is represented by one of Formulae (VIII-1)˜(VIII-6):

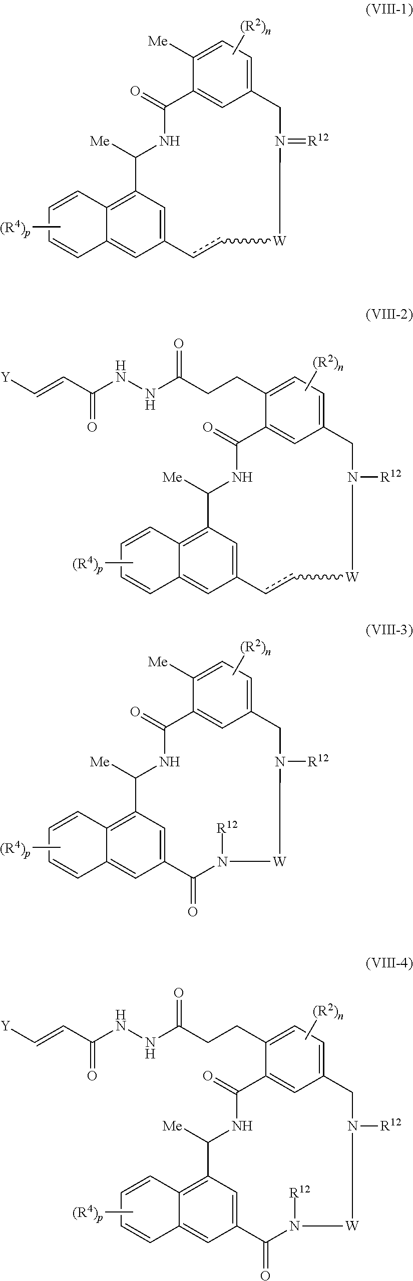

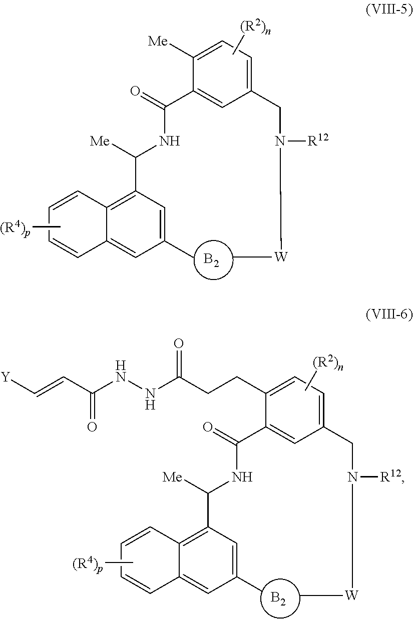

wherein B₂, R², R⁴, R¹², W, n, p, and Y are as previously defined.

In certain embodiments, the compound of Formula (I) is represented by one of Formulae (IX-1)˜(IX-6):

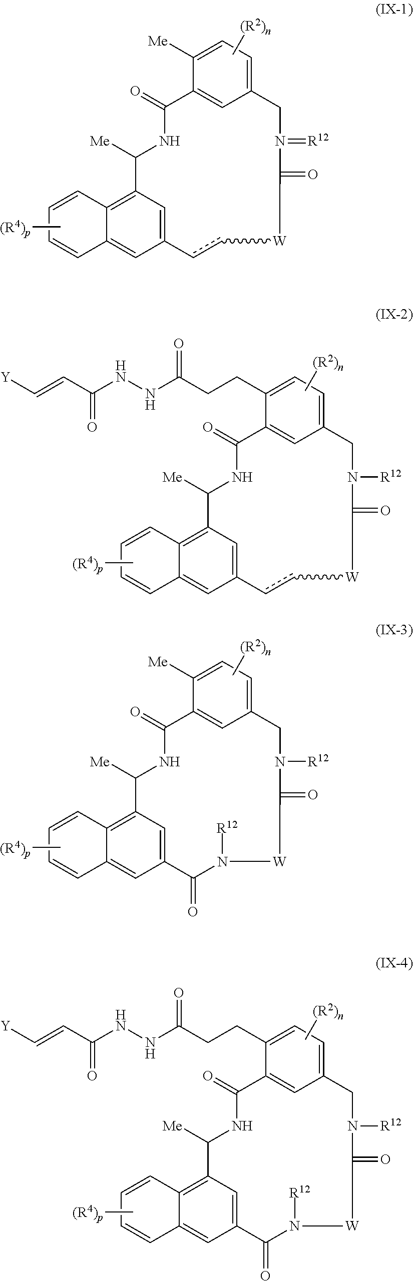

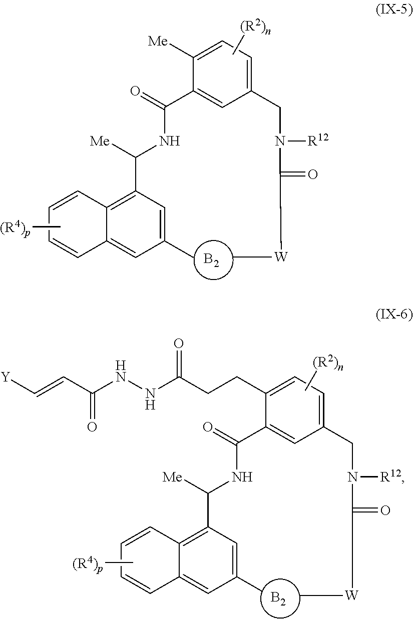

wherein B₂, R², R⁴, R¹², W, n, p, and Y are as previously defined.

In certain embodiments, the compound of Formula (I) is represented by one of Formulae (X-1)˜(X-6):

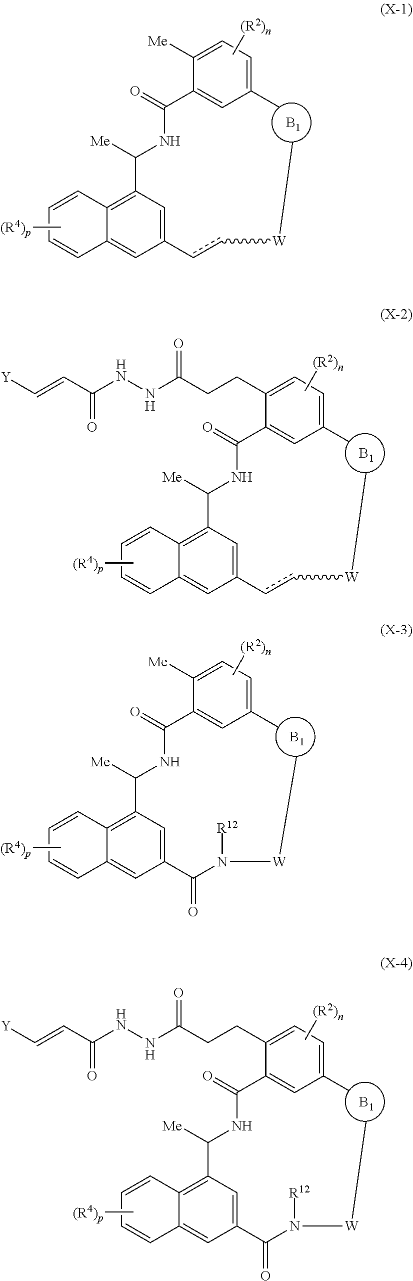

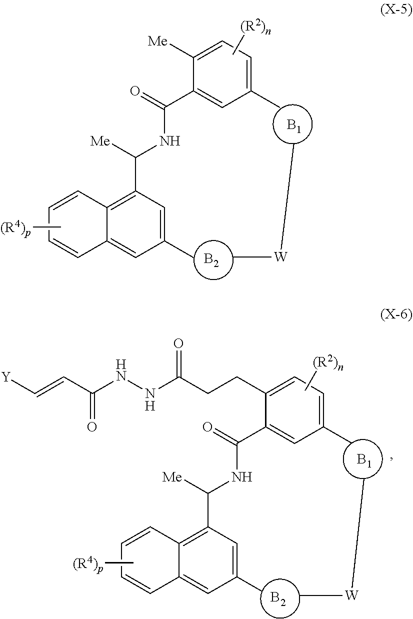

wherein B₁, B₂, R², R⁴, R¹², W, n, p, and Y are as previously defined.

In certain embodiments, the compound of Formula (I) is represented by one of Formulae (XI-1)˜(XI-8):

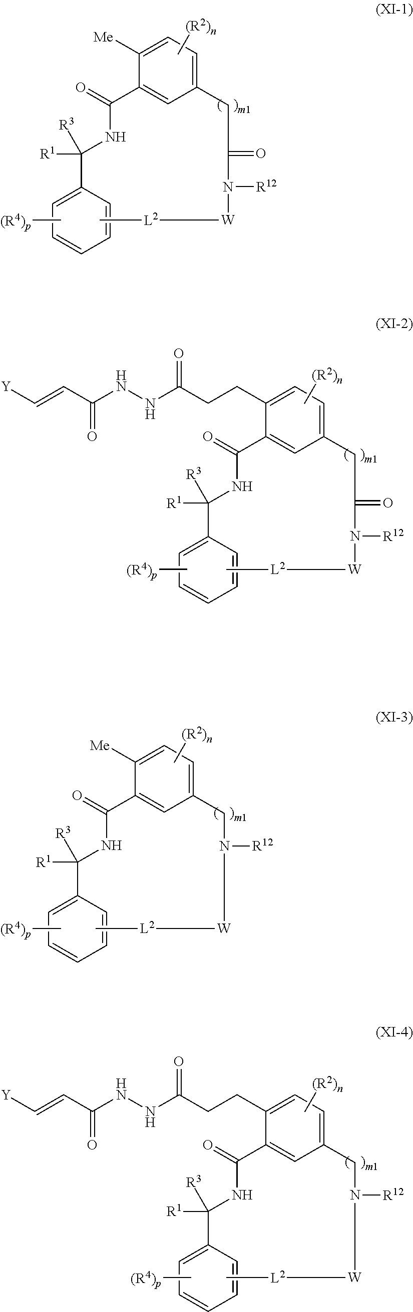

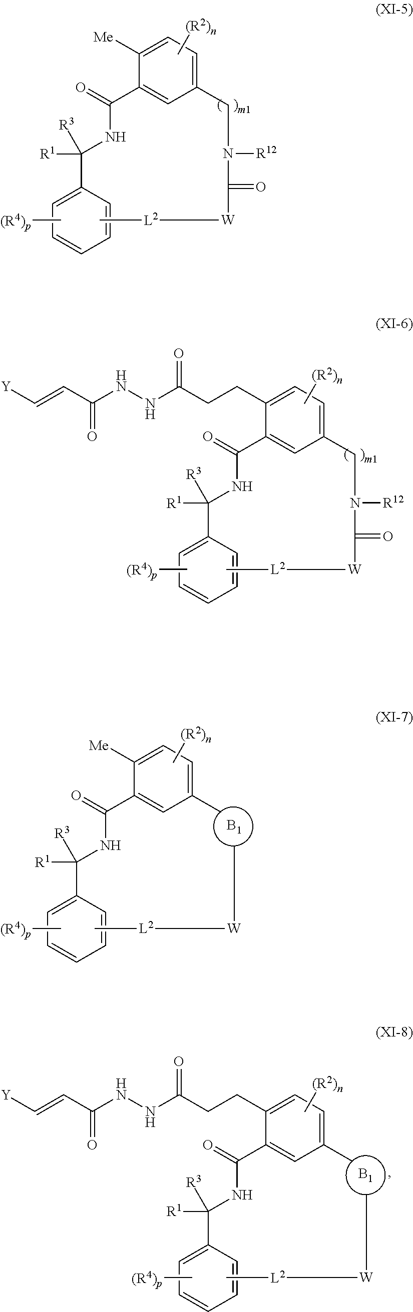

wherein B₁, R¹², R¹, R², R³, R⁴, L², W, m1, n, and p are as previously defined.

In certain embodiments, the compound of Formula (I) is represented by one of Formulae (XII-1)˜(XII-8):

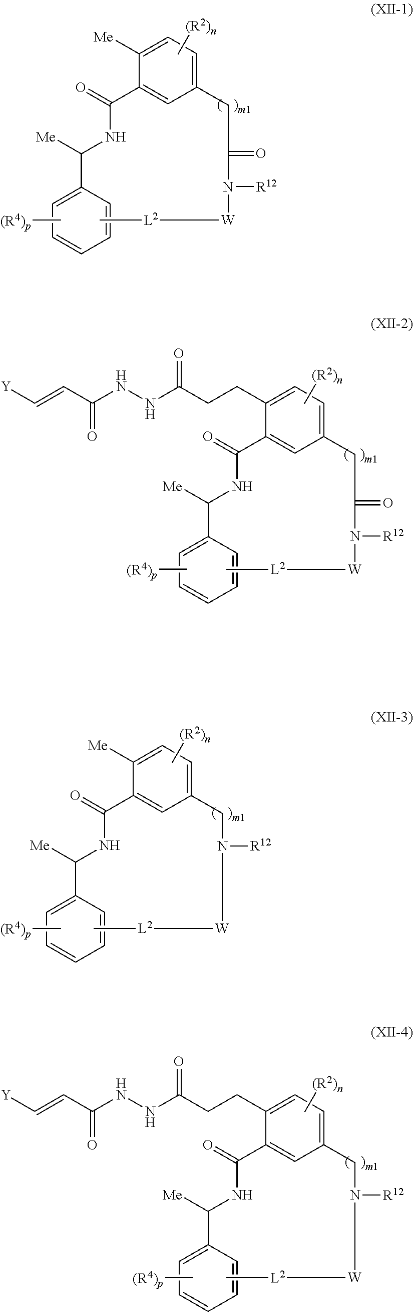

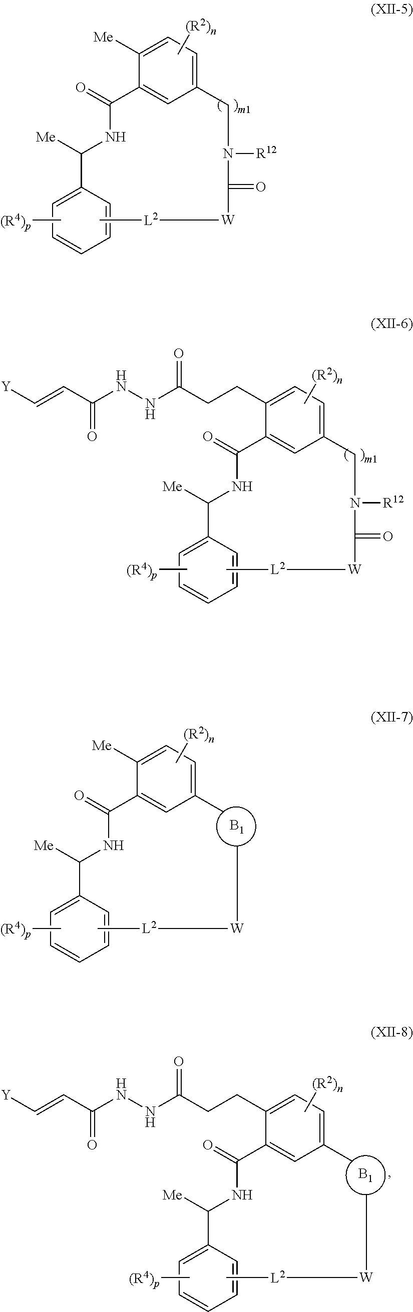

wherein B₁, R¹², R², R⁴, L², W, m1, n, and p are as previously defined.

In certain embodiments, the compound of Formula (I) is represented by Formula (XIII),

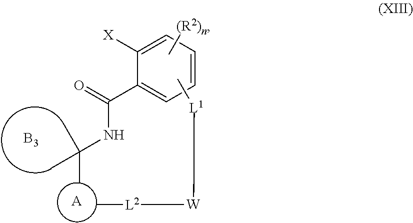

wherein B₃ is selected from the group consisting of optionally substituted —C₃-C₈ cycloalkyl, optionally substituted 3- to 8-membered heterocycloalkyl, and optionally substituted —C₃-C₈ cycloalkenyl, and A, X, R², L¹, L², W and n are as previously defined.

In certain embodiments, the compound of Formula (I) is represented by one of Formulae (XIV-1)˜(XIV-4),

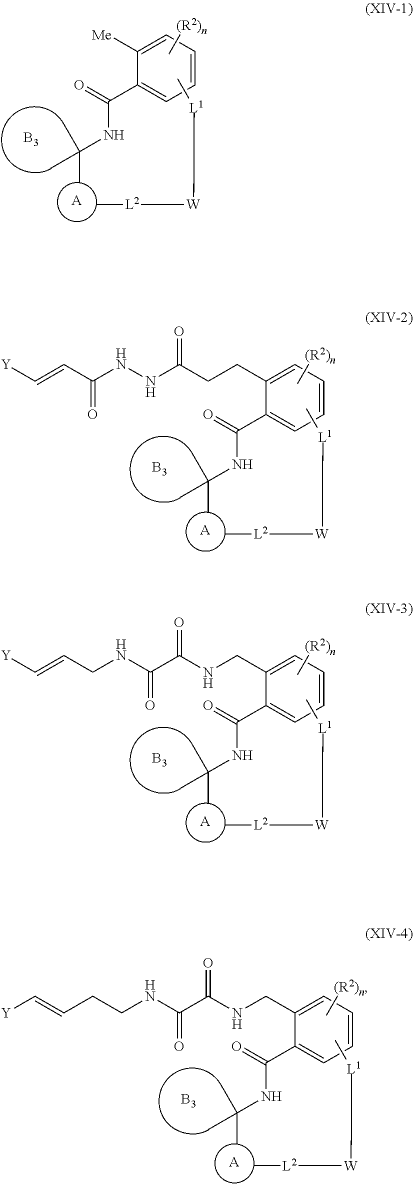

wherein B₃, A, R², L¹, L², W and n are as previously defined.

In certain embodiments, the compound of Formula (I) is represented by one of Formulae (XV-1)˜(XV-8):

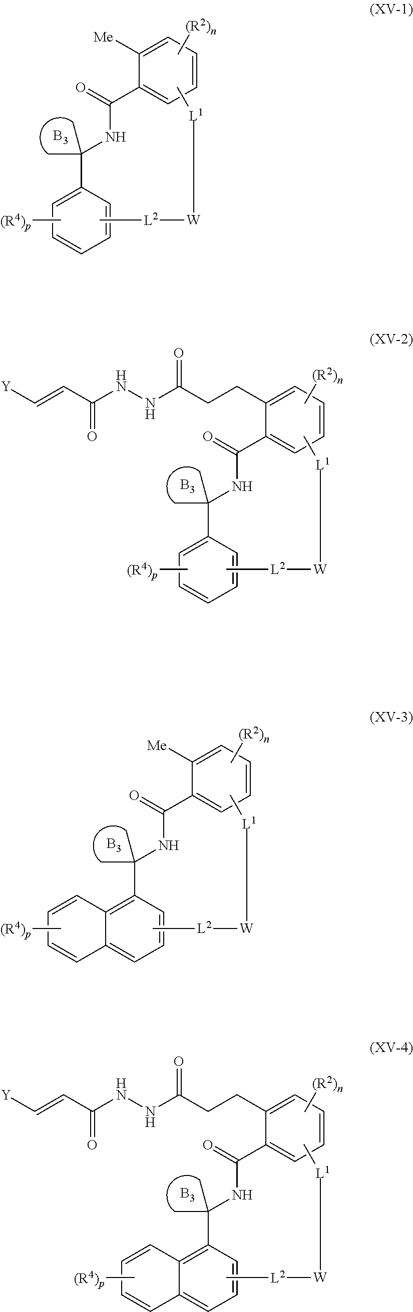

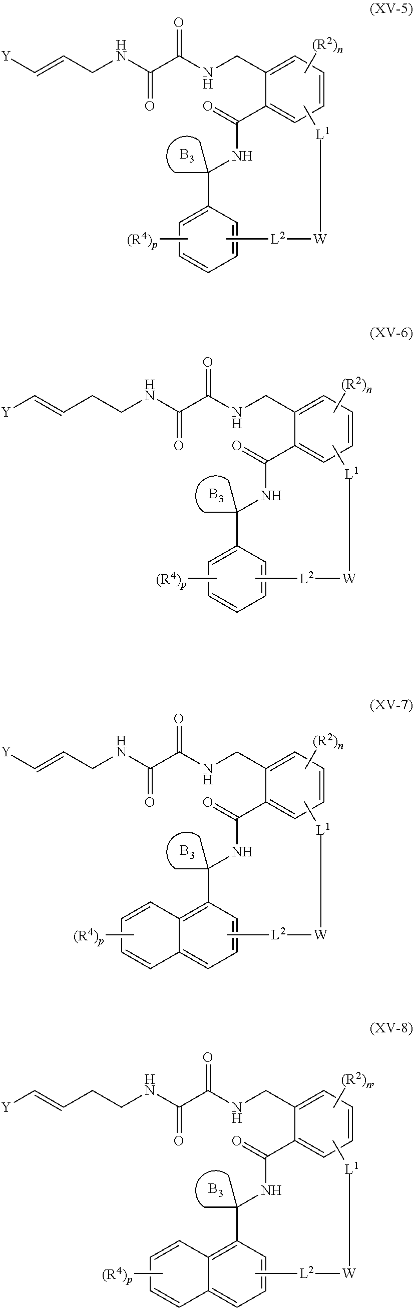

wherein B₃, R², R⁴, L¹, L², W, n, and p are as previously defined.

In certain embodiments, the compound of Formula (I) is represented by one of Formulae (XVI-1)˜(XVI-8):

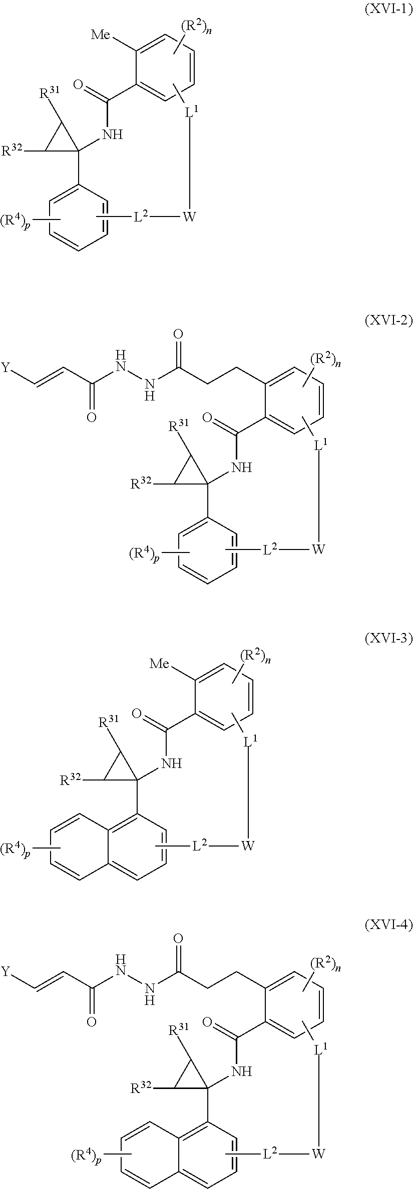

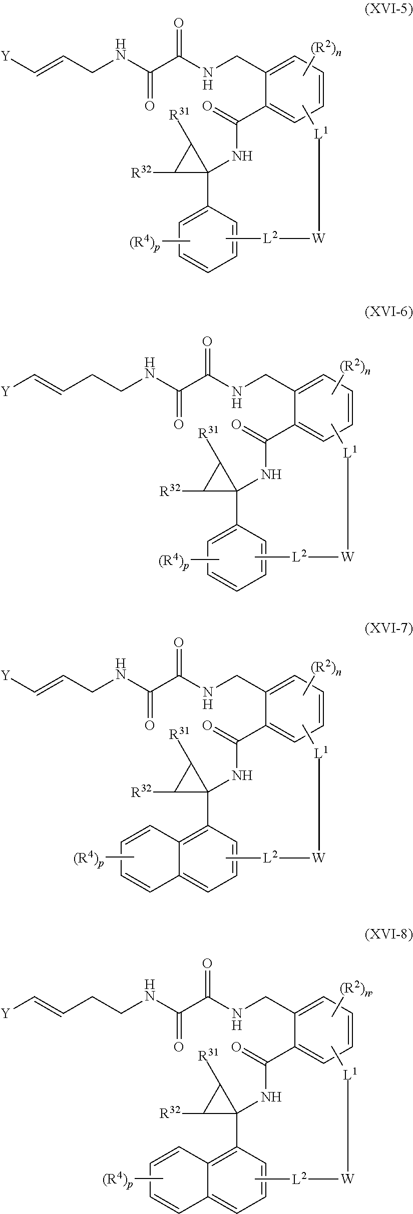

wherein R³¹, R³², R², R⁴, L¹, L², W, n, and p are as previously defined.

In certain embodiments, the compound of Formula (I) is represented by one of Formulae (XVII-1)˜(XVII-8):

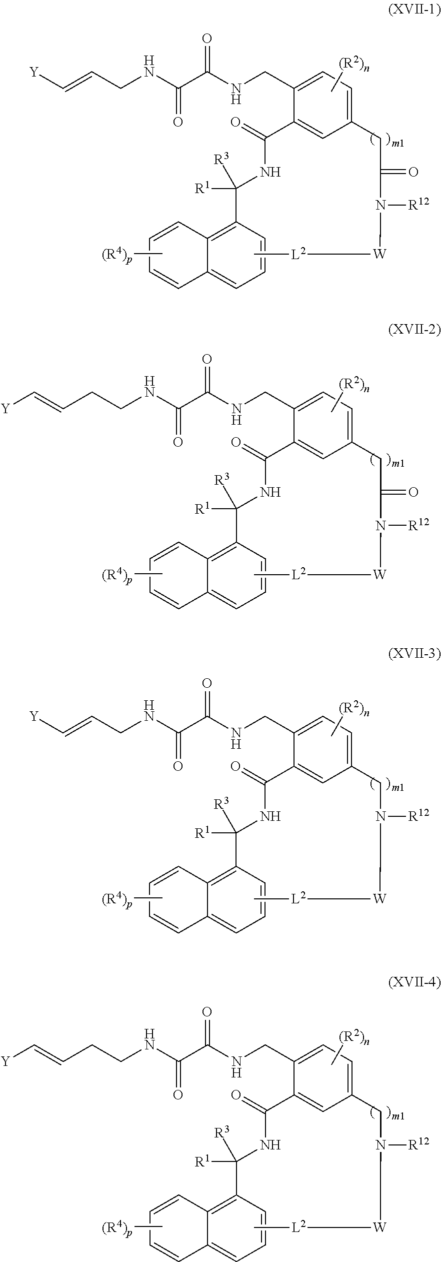

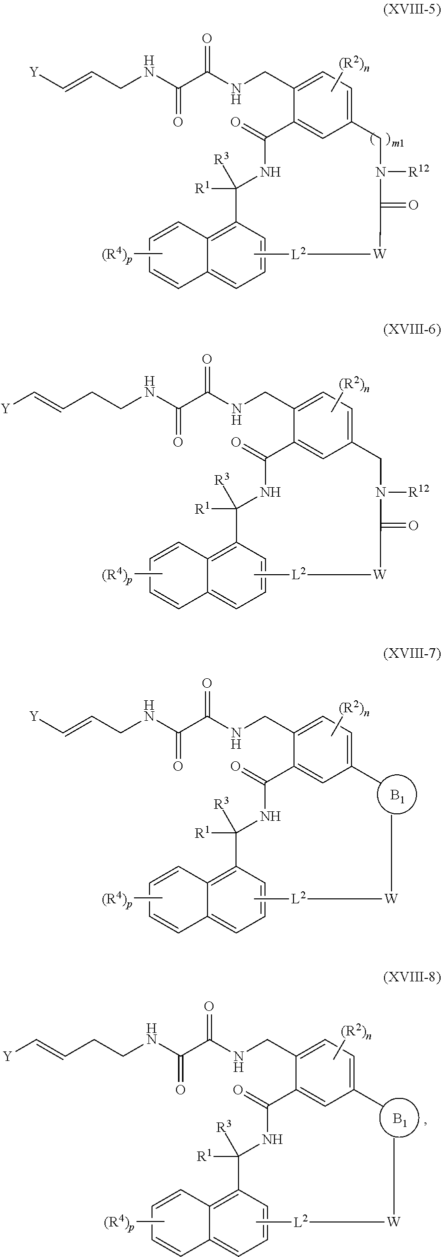

wherein B₁ m1, R¹, R², R³, R⁴, R¹², L², W, n, p, and Y are as previously defined, preferably, R¹ is methyl or —CD₃, and R³ is hydrogen.

In certain embodiments, the compound of Formula (I) is represented by one of Formulae (XVIII-1)˜(XVIII-8):

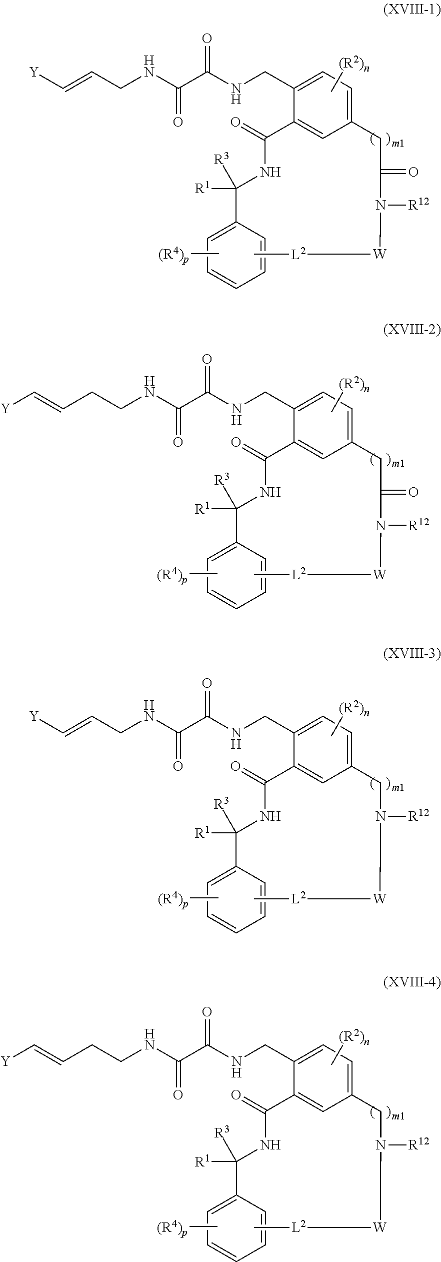

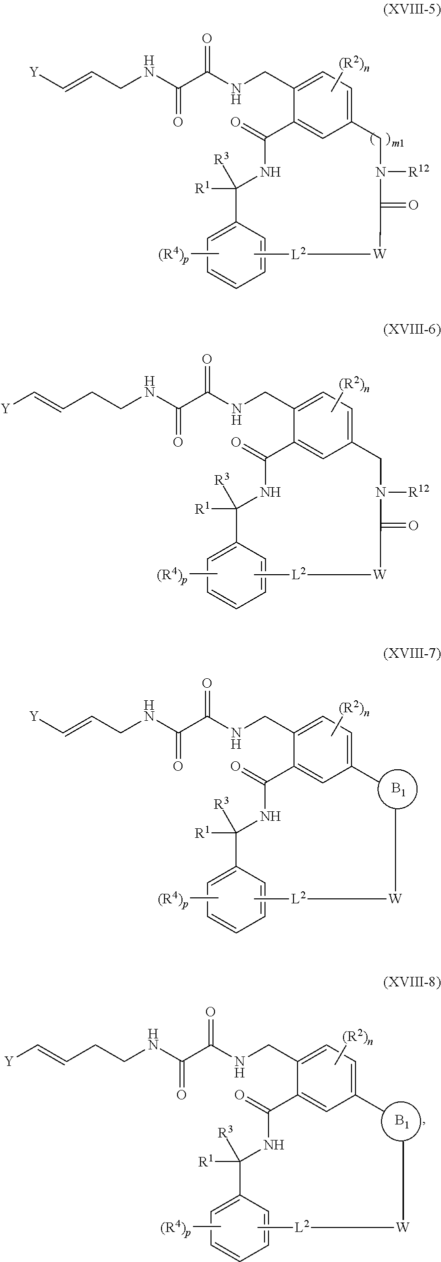

wherein B₁ m1, R¹, R², R³, R⁴, R¹², L², W, n, p, and Y are as previously defined, preferably, R¹ is methyl or —CD₃, and R³ is hydrogen.

The present invention provides a pharmaceutical composition comprising a biologically active compound of the invention for the treatment of coronavirus in a mammal containing an amount of a coronavirus PLpro inhibitor that is effective in treating coronavirus and a pharmaceutically acceptable carrier or expedient.

The present invention provides a method of inhibiting the activity of a coronavirus PLpro, comprising contacting the coronavirus PLpro with an effective amount of a coronavirus PLpro inhibitor compound or agent.

The present invention also provides a method of targeting coronavirus inhibition as a means of treating indications caused by coronavirus related viral infections.

The present invention provides a method of treating or preventing a coronavirus infection in a subject in need thereof, comprising administering to the subject a therapeutically effective amount of a compound or a combination of compounds.

The present invention provides a method of treating or preventing a coronavirus infection in a subject in need thereof, further comprising administering to the subject an additional therapeutic agent selected from the group consisting of a coronavirus protease inhibitor, interferon, viral entry inhibitor, viral maturation inhibitor, inducer of cellular viral RNA sensor, therapeutic vaccine, and agents of distinct or unknown mechanism, and a combination thereof.

The present invention provides a method of reducing viral load in the subject to a greater extent compared to the administering of a compound selected from the group consisting of a coronavirus protease inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

The present invention provides a method resulting in a lower incidence of viral mutation and/or viral resistance than the treatment with a compound selected from the group consisting of a coronavirus protease inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

In certain embodiments, the invention provides a method of treating or preventing a coronavirus infection in a subject, such as a human, in need thereof, comprising the step of administering to the subject a therapeutically effective amount of a compound of Formula (I) or a pharmaceutically acceptable salt thereof. The coronavirus can be an alpha, beta, gamma or delta coronavirus. In certain embodiments, the coronavirus is one which infects humans, such as coronavirus 229E, coronavirus NL63, coronavirus OC43, coronavirus HKU1, SARS-CoV-1, SARS-CoV-2, and MERS-CoV. In certain embodiments, the coronavirus is SARS-CoV-1, SARS-CoV-2, or MERS-CoV. Preferably the coronavirus is SARS-CoV-2.

Embodiments of the present invention provide administration of a compound to a healthy or virus-infected patient, either as a single agent or in combination with (1) another agent that is effective in treating or preventing coronavirus infections, (2) another agent that improves immune response and robustness, or (3) another agent that reduces inflammation and/or pain.

In a further aspect, this invention provides for a method of treating a respiratory disorder in a subject in need thereof, comprising administering to the subject a therapeutically effective amount of a compound of Formula (I) or a pharmaceutically acceptable salt thereof. Such respiratory disorders include, but are not limited to, an acute airway disease or a chronic airway disease. Examples of such respiratory disorders include acute asthma, lung disease secondary to environmental exposures, acute lung infection, and chronic lung infection.

It will be appreciated that the description of the present invention herein should be construed in congruity with the laws and principles of chemical bonding. In some instances, it may be necessary to remove a hydrogen atom in order to accommodate a substituent at any given location.

It will be yet appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing coronavirus.

It should be understood that the compounds encompassed by the present invention are those that are suitably stable for use as pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ,-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy is C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH— heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, —CO₂-C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)— heterocycloalkyl, —NHCO₂—C₁-C₁₂-alkyl, —NHCO₂—C₂-C₈-alkenyl, —NHCO₂-C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH— heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂—C₁-C₁₂-alkyl, —SO₂-C₂-C₈-alkenyl, —SO₂-C₂-C₈-alkynyl, —SO₂-C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂-C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxycarbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl(trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, NJ (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)-for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Combination and Alternation Therapy

The compounds of the present invention may be used in combination with one or more antiviral therapeutic agents or anti-inflammatory agents useful in the prevention or treatment of viral diseases or associated pathophysiology. Thus, the compounds of the present invention and their salts, solvates, or other pharmaceutically acceptable derivatives thereof, may be employed alone or in combination with other antiviral or anti-inflammatory therapeutic agents. The compounds herein and pharmaceutically acceptable salts thereof may be used in combination with one or more other agents which may be useful in the prevention or treatment of respiratory disease, inflammatory disease, autoimmune disease, for example; anti-histamines, corticosteroids, (e.g., fluticasone propionate, fluticasone furoate, beclomethasone dipropionate, budesonide, ciclesonide, mometasone furoate, triamcinolone, flunisolide), NSAIDs, Ieukotriene modulators (e.g., montelukast, zafirlukast.pranlukast), tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (Iysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-ethylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-lgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents), suitable anti-infective agents including antibiotic agents, antifungal agents, antheimintic agents, antimalarial agents, antiprotozoal agents, antituberculosis agents, and antiviral agents, including those listed at https://www.drugs.com/drug-class/anti-infectives.html. In general, combination therapy is typically preferred over alternation therapy because it induces multiple simultaneous stresses on the virus.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

Although the invention has been described with respect to various preferred embodiments, it is not intended to be limited thereto, but rather those skilled in the art will recognize that variations and modifications may be made therein which are within the spirit of the invention and the scope of the appended claims.

### Antiviral Activity

In certain embodiments, the present invention provides a method of treating or preventing a viral infection in a subject in need thereof, comprising administering to the subject a therapeutically effective amount of a compound of Formula (I) or a pharmaceutically acceptable salt thereof. The viral infection is preferably a coronavirus infection. In certain embodiments, the coronavirus is SARS-CoV-1, SARS-CoV-2, or MERS-CoV. Preferably the coronavirus is SARS-CoV-2.

A viral inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, viral infections are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). A therapeutically effective amount of the compound described above may range, for example, from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Abbreviations

Abbreviations which may be used in the descriptions of the scheme and the examples that follow are: Ac for acetyl; AcOH or HOAc for acetic acid; ACN or MeCN or CH₃CN for acetonitrile; BF₃·OEt₂ for boron trifluoride diethyl etherate; Boc₂O for di-tert-butyl-dicarbonate; Boc for t-butoxycarbonyl; Bz for benzoyl; Bn for benzyl; t-BuOK for potassium tert-butoxide; Brine for sodium chloride solution in water; CbzCl or Cbz-Cl for benzyl chloroformate; CDI for carbonyldiimidazole; DCM or CH₂Cl₂ for dichloromethane; CH₃ for methyl; (COCl)₂ for oxalyl chloride; Cl₂CHCN for dichloroacetonitrile; Cs₂CO₃ for cesium carbonate; CuCl for copper (I) chloride; CuI for copper (I) iodide; CuSO₄ for copper (II) sulfate; dba for dibenzylidene acetone; DBU for 1,8-diazabicyclo[5.4.0]-undec-7-ene; DCC for N,N′-dicyclohexylcarbodiimide; DCE for 1,2-dichloroethane; DIBAL-H for diisobutylaluminum hydride; DIPEA or (i-Pr)₂EtN for N,N,-diisopropylethyl amine; DMP or Dess-Martin periodinane for 1,1,2-tris(acetyloxy)-1,2-dihydro-1,2-benziodoxol-3-(1H)-one; DMAP for 4-dimethylamino-pyridine; DME for 1,2-dimethoxyethane; DMF for N,N-dimethylformamide; DMSO for dimethyl sulfoxide; EDC for 1-ethyl-3-(3-dimethylaminopropyl)carbodiimide; EtOAc for ethyl acetate; EtOH for ethanol; Et₂O for diethyl ether; H₂ for hydrogen, HATU for O-(7-azabenzotriazol-2-yl)-N,N,N′,N′,-tetramethyluronium Hexafluoro-phosphate; HCl for hydrogen chloride; K₂CO₃ for potassium carbonate; n-BuLi for n-butyl lithium; KHMDS for potassium bis(trimethylsilyl)amide; IBX for 2-iodoxybenzoic acid; In for indium; LDA for lithium diisopropylamide; Li for lithium; LiBH₄ for lithium borohydride; LiBr for lithium bromide; LiHMDS for lithium bis(trimethylsilyl)amide; LiOH for lithium hydroxide; LiTMP for lithium 2,2,6,6-tetramethyl-piperidinate; MeOH for methanol; Mg for magnesium; MOM for methoxymethyl; Ms for mesyl or —SO₂—CH₃; NaHMDS for sodium bis(trimethylsilyl)amide; NaCl for sodium chloride; NaBH₄ for sodium borohydride; NaBH(OAc)₃ for sodium triacetoxyborohydride; NaH for sodium hydride; NaHCO₃ for sodium bicarbonate or sodium hydrogen carbonate; Na₂CO₃ sodium carbonate; NaOH for sodium hydroxide; Na₂SO₄ for sodium sulfate; NaHSO₃ for sodium bisulfite or sodium hydrogen sulfite; Na₂S₂O₃ for sodium thiosulfate; NBS for N-bromosuccinimide; NH₃ for ammonia; NH₄OH for ammonium hydroxide; NH₂NH₂ for hydrazine; NH₄Cl for ammonium chloride; Ni for nickel; NMM for N-methylmorpholine; n-PrOH for 1-propanol; OH for hydroxyl; OsO₄ for osmium tetroxide; OTf for triflate; PPA for polyphophoric acid; PTSA or PTSOH for p-toluenesulfonic acid; PPTS for pyridinium p-toluenesulfonate; SiliaMetS DMT for the silica-bound equivalent of 2,4,6-trimercaptotriazine (trithiocyanuric acid, TMT); SO₃ for sulfur trioxide; TBAF for tetrabutylammonium fluoride; TEA or Et₃N or NEt₃ for triethylamine; TFA for trifluoroacetic acid; TFAA for trifluoroacetic anhydride; THF for tetrahydrofuran; T₃P for propylphosphonic anhydride; TPP or PPh₃ for triphenyl-phosphine; Tos or Ts for tosyl or —SO₂—C₆H₄CH₃; Ts₂O for tolylsulfonic anhydride or tosyl-anhydride; TsOH for p-tolylsulfonic acid; Pd for palladium; Pd/C for palladium on carbon; Ph for phenyl; Pd₂(dba)₃ for tris(diben-zylideneacetone) dipalladium (0); Pd(PPh₃)₄ for tetrakis(triphenylphosphine)-palladium (0); PdCl₂(PPh₃)₂ for trans-dichlorobis-(triphenylphosphine)palladium (II); PdCl₂(dppf) for [1,1′-bis(diphenylphosphino)ferrocene]palladium(II) dichloride; Pd(TFA)₂ for palladium(II) trifluoroacetate; for Pt for platinum; Rh for rhodium; rt for room temperature; Ru for ruthenium; TBS for tert-butyl dimethylsilyl; TMS for trimethylsilyl; or TMSCl for trimethylsilyl chloride; TMSOTf for trimethylsilyl trifluoromethanesulfonate; Zhan 1B cat. for dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II).

### Synthetic Methods

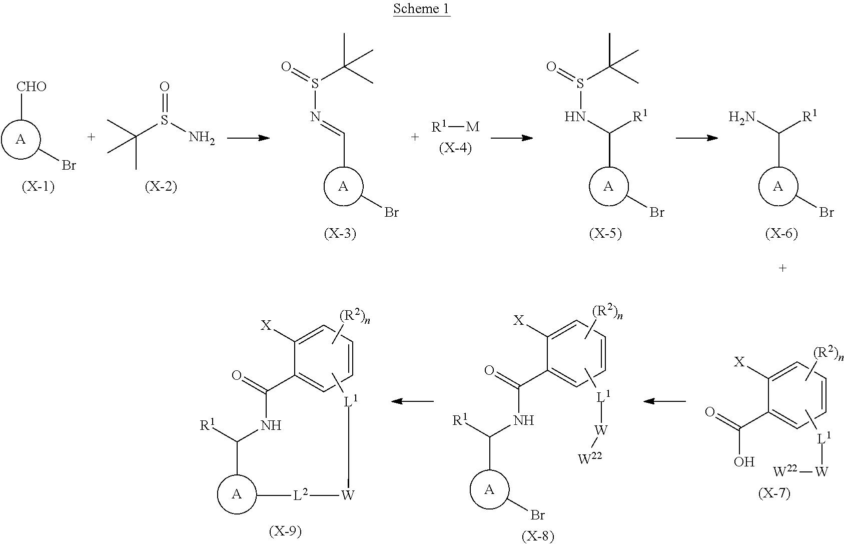

Scheme 1 illustrates a general method to prepare the compound (X-9) of formula I from the aldehyde (X-1), wherein A is as previously defined. Condensation of aldehyde (X-1) with tert-butanesulfinamide (X-2) affords the imine (X-3), which was converted to sulfinamide (X-5) by nucleophilic addition of an organometallic reagent X-4, wherein R¹ is as previously defined and M is —MgBr, —MgCl, —Li, —In, or —Zn. Deprotection of sulfinamide (X-5) under acidic conditions provides the chiral primary amine (X-6). Amide coupling of amine (X-4) with carboxylic acid (X-7) gives the amide (X-8), whererin X, R², L¹, W, and n are as previously defined and W²² is the corresponding functionality to form the macrocycle linkage L² that is as previously defined. Macrocyclization of (X-8) via intramolecular Suzuki coupling, intramolecular Heck reaction, intramolecular S_{N}Ar, or intramolecular Buchwald-Hartwig coupling reacts W² with —Br to form macrocyclic compound (X-9), wherein L² is as previously defined. These widely-used macrocyclication approaches have been reviewed in the literature (Marsault, E. et al. “Macrocycles Are Great Cycles: Applications, Opportunities, and Challenges of Synthetic Macrocycles in Drug Discovery” J. Med. Chem. 2011, 54, 7, 1961-2004). The application of intramolecular Suzuki coupling in synthesis of macrocyclic compounds has been reported in the literature (Li, H. et al. “Synthesis of Bis-Macrocyclic HCV Protease Inhibitor MK-6325 via Intramolecular sp²-sp³ Suzuki-Miyaura Coupling and Ring Closing Metathesis” Org. Lett. 2015, 17, 6, 1533-1536). The application of intramolecular Heck reaction in synthesis of macrocyclic compounds have been reported in the literature (Zhang, W. “Heck macrocyclization in natural product total synthesis” Nat. Prod. Rep., 2021, 38, 1109).

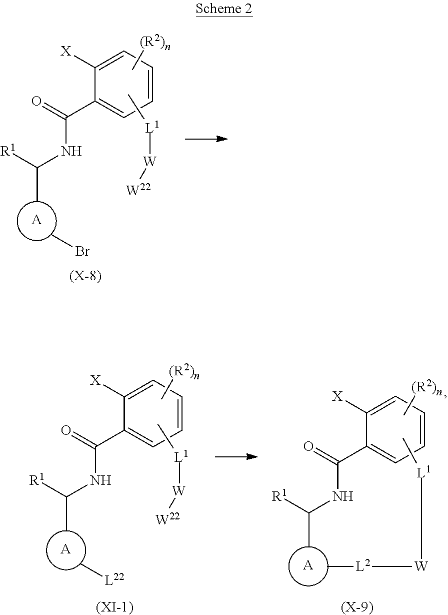

Scheme 2 illustrates another general method to synthesize the compound (X-9) of formula (I), wherein A, R¹, X, L¹, L², W, R² and n are as previously defined. The bromide of amide (X-8) is converted to L²² through Pd-catalyzed cross couplings or Cu-catalyzed ullmann couplings, wherein W²² is as previously defined and L²² is the corresponding functionality to form the macrocycle linkage L² that is as previously defined. Macrocyclization of (XI-1) via ring-closing metathesis, intramolecular click chemistry, or macrolactamization reacts W² with L²² to form L², thus providing macrocyclic compound (X-9). These widely-used macrocyclication approaches have been reviewed in the literature (Marsault, E. et al. “Macrocycles Are Great Cycles: Applications, Opportunities, and Challenges of Synthetic Macrocycles in Drug Discovery” J. Med. Chem. 2011, 54, 7, 1961-2004). For example, the application of ring closing metathesis in synthesis of macrocyclic compounds has been reported in the literature (Yu, M. et al. “Ring-Closing Metathesis in Pharmaceutical Development: Fundamentals, Applications, and Future Directions” Org. Process Res. Dev. 2018, 22, 8, 918-946; Damalanka, V. C. et al. “Design, synthesis, and evaluation of a novel series of macrocyclic inhibitors of norovirus 3CL protease” European Journal of Medicinal Chemistry, Volume 127, 15 Feb. 2017, Pages 41-61). The application of intramolecular click chemistry in synthesis of macrocyclic compounds has been reported in the literature (Weerawarna, P. M. et al. “Structure-based design and synthesis of triazole-based macrocyclic inhibitors of norovirus protease: Structural, biochemical, spectroscopic, and antiviral studies” European Journal of Medicinal Chemistry, Volume 119, 25 Aug. 2016, Pages 300-318). The application of macrolactamization in synthesis of macrocyclic compounds has been reported in the literature (Li, B. et al. “Exploratory Process Development of Lorlatinib” Org. Process Res. Dev. 2018, 22, 1289-1293).

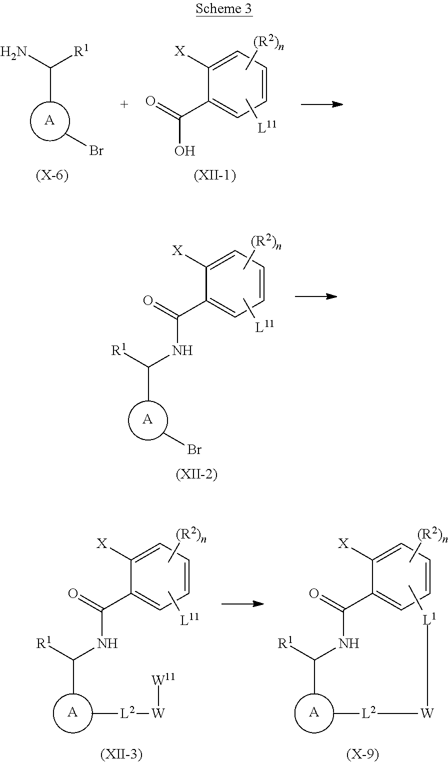

Scheme 3 illustrates another general method to synthesize the compound (X-9) of formula (I), wherein A, R¹, X, L¹, L², W, R² and n are as previously defined. The amide coupling of amine (X-6) with carboxylic acid (XII-1) affords amide (XII-2), wherein L¹¹ is the corresponding functionality to form the macrocycle linkage L¹ that is as previously defined. The L2-W-W11 tether of (XII-3) is introduced by using bromide of amide (XII-2) as a synthetic handle, wherein L2 and W are as previously defined and W¹¹ is the corresponding functionality to form the macrocycle linkage L¹ that is as previously defined is converted. Macrocyclization of (XII-3) via the methods mentioned above reacts W¹¹ with L¹¹ to form L¹, thus providing macrocyclic compound (X-9).

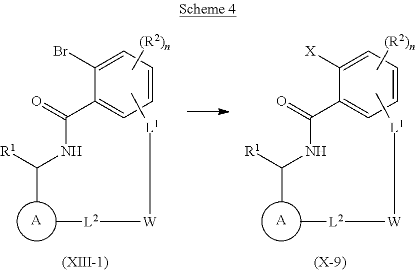

Scheme 4 illustrates another general method to synthesize compound (X-9) of formula (I), wherein A, R¹, X, L¹, L², W, R² and n are as previously defined. The compound (XIII-1) is prepared by the method described in previous Schemes. The X of (X-9) could be introduced in a late stage by using the bromide of (XIII-1) as the synthetid handle via Pd-catalyzed cross couplings.

### EXAMPLES

The compounds and processes of the present invention will be better understood in connection with the following examples, which are intended as an illustration only and not limiting of the scope of the invention. Starting materials were either available from a commercial vendor or produced by methods well known to those skilled in the art.

### Example 1

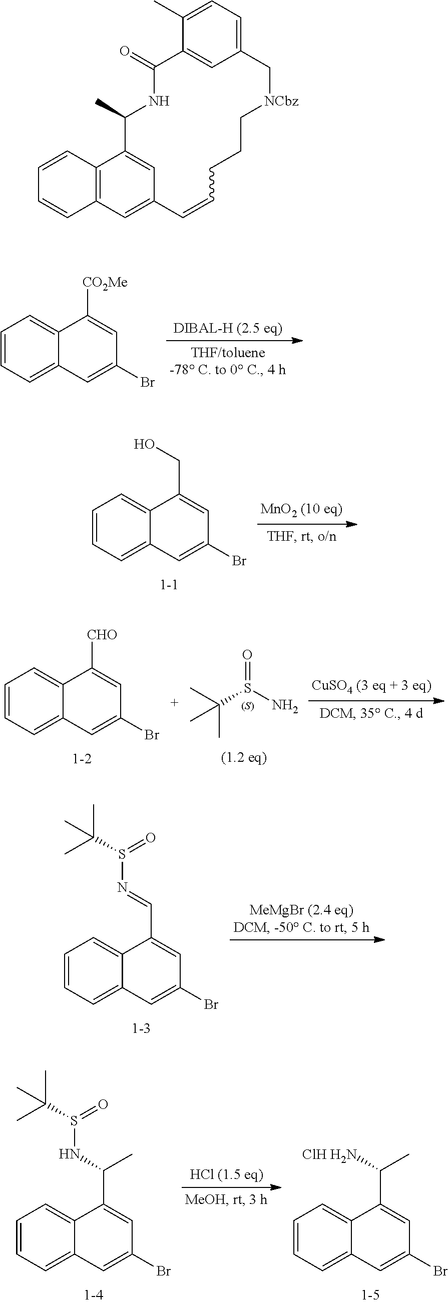

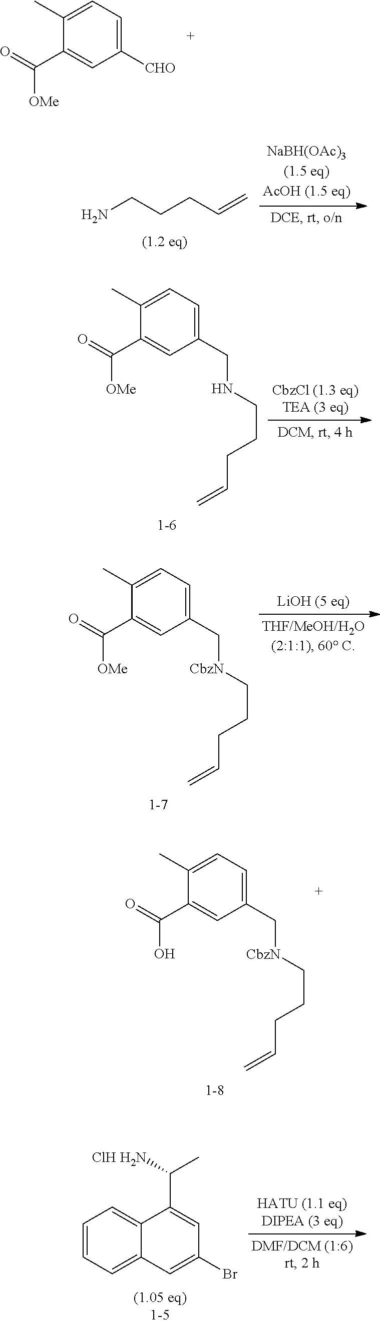

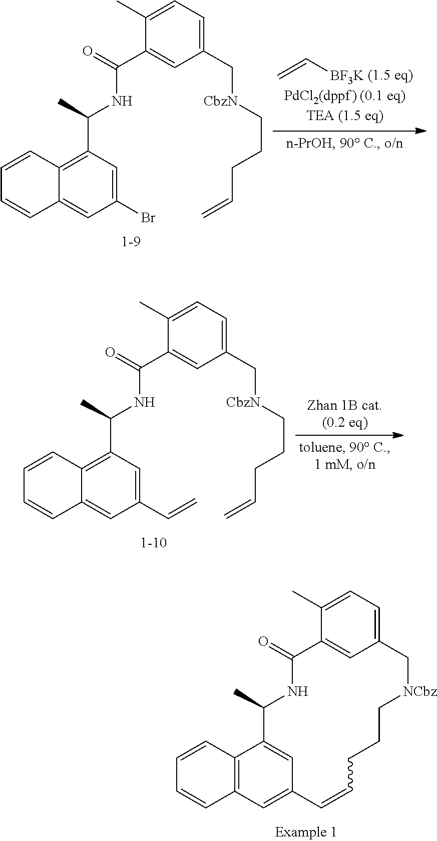

### Step 1-1:

A solution of methyl 3-bromo-1-naphthoate (5 g, 18.86 mmol) in THF (24 ml) was treated with a 1 M solution of DIBAL-H (50 ml, 50.0 mmol) in Toluene (50 ml) at −78° C. The reaction was gradually warmed to 0° C. and stirred for 4 h. The reaction was quenched with a saturated solution of potassium sodium tartrate slowly at 0° C. The mixture was stirred at room temperature overnight to form a clear biphasic solution. The aqueous layer was extracted with ethyl acetate over 3 times. The combined organic layer was dried over sodium sulfate, filtered and concentrated in vacuo. The crude was dried under high vacuum to give the desired product (4.4 g, 18.56 mmol, 98% yield) as an off-white solid. ESI MS m/z=219.04, 221.06 [M+H−H₂O]⁺.

### Step 1-2:

A solution of the compound from Step 1-1 (4.4 g, 18.56 mmol) in THF (60 ml) was treated with manganese dioxide (8 g, 92 mmol). The reaction was stirred at room temperature overnight. The mixture was filtered and concentrated in vacuo. The crude was added to a 80 g silica gel column and eluted by ethyl acetate/cyclohexane from 0% to 30% to give the desired product (3.5 g, 14.89 mmol, 80% yield) as a yellow solid. ESI MS m/z=232.86, 234.74 [M−H]⁻.

### Step 1-3:

A solution of the compound from Step 1-2 (3.5 g, 14.89 mmol) and (S)-2-methylpropane-2-sulfinamide (2.2 g, 18.15 mmol) in CH₂Cl₂ (30 ml) was treated with copper(II) sulfate (10.6 g, 66.4 mmol) at room temperature. The reaction was stirred at room temperature for 4d. The mixture was filtered and rinsed with acetone. The filtrate was concentrated in vacuo. The crude was added to a 80 g silica gel column and eluted by ethyl acetate/cyclohexane from 0% to 100% to give the desired product (3.57 g, 10.55 mmol, 71% yield) as an off-white solid. ESI MS m/z=338.24, 340.09 [M+H]⁺.

### Step 1-4:

A solution of the compound from Step 1-3 (3.57 g, 10.55 mmol) in CH₂Cl₂ (50 ml) was treated with methylmagnesium bromide (8.2 ml, 24.60 mmol) dropwise at −50° C. The reaction was stirred at −50° C. for 1 h and then allowed to slowly warm to room temperature. The reaction was stirred at room temperature for 4 h. The mixture was quenched with a saturated solution of ammonium chloride. The aqueous layer was extracted with dichloromethane over 3 times. The combined organic layer was dried over sodium sulfate, filtered and concentrated in vacuo. The crude was added to a 80 g silica gel column and eluted by ethyl acetate/cyclohexane from 0% to 100% to the desired product (2.9 g, 8.19 mmol, 78% yield, >15:1 dr) as a white foam. ESI MS m/z=354.11, 356.08 [M+H]⁺.

### Step 1-5:

A solution of the compound from Step 1-4 (2.9 g, 8.19 mmol) in MeOH (22 ml) was treated with 4N HCl in dioxane (3 ml, 12.00 mmol) dropwise at room temperature. The reaction was stirred at room temperature for 3 h. The solution was concentrated in vacuo and dried under high vacuum to give the desired product (2.22 g, 7.75 mmol, 95% yield) as a white solid. ESI MS m/z=233.10, 235.10 [M+H]⁺.

### Step 1-6:

A solution of methyl 5-formyl-2-methylbenzoate (1.6 g, 8.98 mmol) and pent-4-en-1-amine (0.9 g, 10.57 mmol) in DCE (40 ml) was treated with AcOH (0.76 ml, 13.28 mmol) and sodium triacetoxyborohydride (2.8 g, 13.21 mmol). The reaction was stirred at room temperature overnight. The reaction was quenched with a saturated solution of sodium bicarbonate. The aqueous layer was extracted with dichloromethane over 3 times. The combined organic layer was dried over sodium sulfate, filtered and concentrated in vacuo. The crude was added to a 40 g silica gel column and eluted by dichloromethane/methanol from 0% to 20% to give the desired product (1.37 g, 5.54 mmol, 62% yield) as a yellow oil. ESI MS m/z=248.19 [M+H]⁺.

Step 1-7: A solution of the compound from Step 1-6 (1.37 g, 5.54 mmol) in CH₂Cl₂ (20 ml) was treated with TEA (2.4 ml, 17.22 mmol) and Cbz-Cl(1 ml, 7.01 mmol). The reaction was stirred at room temperature for 4 h. The reaction was quenched with a saturated solution of sodium bicarbonate. The mixture was stirred at room temperature for 1 h. The aqueous layer was extracted with dichloromethane over 3 times. The combined organic layer was dried over sodium sulfate, filtered and concentrated in vacuo. The crude was added to a 40 g silica gel column and eluted by ethyl acetate/cyclohexane from 0% to 30% to give the desired product (1.88 g, 4.93 mmol, 89% yield) as a colorless oil. ESI MS m/z=382.29 [M+H]⁺.

### Step 1-8:

A solution of the compound from Step 1-7 (263 mg, 0.689 mmol) in THF (2 ml) and Water (1 ml) was treated with LiOH (93 mg, 3.88 mmol). The reaction was stirred at 60° C. for 1 h. The reaction was acidified with 1N HCl (3.8 ml, 3.80 mmol) at room temperature. The aqueous layer was extracted with ethyl acetate over 3 times. The combined organic layer was dried over sodium sulfate, filtered and concentrated in vacuo to give the desired product (252 mg, 0.686 mmol, 99% yield) as a clearless syrup. ESI MS m/z=366.19 [M−H]⁻.

### Step 1-9:

A suspension of the compound from Step 1-5 (295 mg, 1.029 mmol) and the compound from Step 1-8 (359 mg, 0.977 mmol) in DMF (0.5 ml) and DCM (3 ml) was treated with HATU (410 mg, 1.078 mmol) and DIPEA (500 μl, 2.86 mmol). The reaction was stirred at room temperature for 2 h. The reaction was quenched with a saturated solution of sodium bicarbonate. The aqueous layer was extracted with dichloromethane over 3 times. The combined organic layer was dried over sodium sulfate, filtered and concentrated in vacuo. The crude was added to a 40 g silica gel column and eluted by ethyl acetate/cyclohexane from 0% to 30% to give the desired product (540 mg, 0.901 mmol, 92% yield) as a white foam. ESI MS m/z=599.18, 601.16 [M+H]⁺.

### Step 1-10:

A solution of the compound from Step 1-9 (540 mg, 0.901 mmol) and trifluoro(vinyl)-14-borane, potassium salt (184 mg, 1.374 mmol) in 1-Proponal (5 ml) was treated with PdCl₂(dppf) (71 mg, 0.097 mmol) and TEA (200 μl, 1.435 mmol) under N₂. The mixture was degassed and backfilled with N₂ over 3 times. The reaction was warmed to 90° C. and stirred overnight. The reaction was concentrated in vacuo. The crude was added to a 40 g silica gel column and eluted by ethyl acetate/cyclohexane from 0% to 25% to give the desired product (338 mg, 0.618 mmol, 69% yield) as an off-white foam. ESI MS m/z=547.34 [M+H]⁺.

### Step 1-11:

A solution of the compound from Step 1-10 (307 mg, 0.562 mmol) in Toluene (500 ml) was treated with Zhan 1B cat. (84 mg, 0.114 mmol) under N₂. The mixture was degassed and backfilled with N₂ through freeze-pump-thaw over 3 times at −78° C. The reaction was warmed to 90° C. and stirred overnight. The mixture was concentrated in vacuo. The crude was added to a 24 g silica gel column and eluted by ethyl acetate/cyclohexane from 0% to 100% to give Example 1 (27 mg, 0.052 mmol, 9% yield) as an off-white solid. ESI MS m/z=519.26 [M+H]⁺.

### Example 2

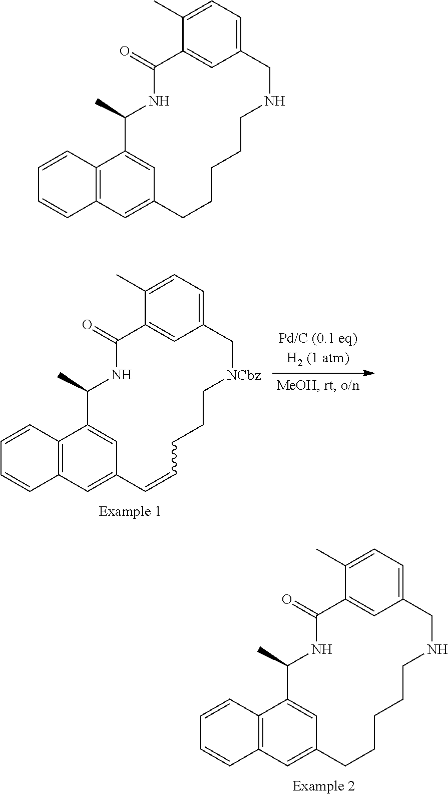 [SMILES: Cc1ccc2cc1C(=O)N[C@H](C)c1cc(cc3ccccc13)CCCCCNC2 ; Cc1ccc2cc1C(=O)N[C@H](C)c1cc(cc3ccccc13)C=CCCCN(C(=O)OCc1ccccc1)C2 ; Cc1ccc2cc1C(=O)N[C@H](C)c1cc(cc3ccccc13)CCCCCNC2]

### Step 2-1:

A solution of the compound from Step 1-11(25 mg, 0.048 mmol) in MeOH (2 ml) was treated with Pd—C (4 mg, 3.76 μmol) under H₂ (1 atm). The reaction was stirred at room temperature overnight. The mixture was filtered through celite and rinsed with methanol over 3 times. The filtrate was concentrated in vacuo. The crude was added to a 4 g silica gel column and eluted by methanol/dichloromethane from 0% to 100% to Example 2 (6 mg, 0.016 mmol, 32% yield) as a white solid. ESI MS m/z=387.36 [M+H]⁺.

TABLE 1
The following examples were prepared employing similar protocol as described above.

| Example | Structure | MS |
|---|---|---|
| 3 | 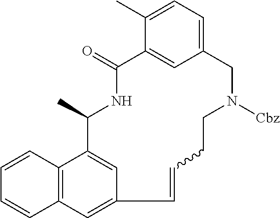 [SMILES: Cc1ccc2cc1C(=O)N[C@H](C)c1cc(cc3ccccc13)C=CCCN(C(=O)OCc1ccccc1)C2] | 505.29 [M + H]⁺ |
| 4 | 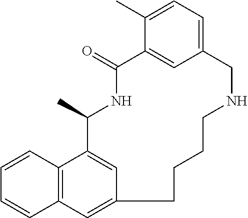 [SMILES: Cc1ccc2cc1C(=O)N[C@H](C)c1cc(cc3ccccc13)CCCCNC2] | 373.29 [M + H]⁺ |
| 5 | 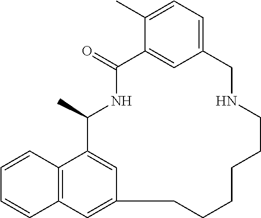 [SMILES: Cc1ccc2cc1C(=O)N[C@H](C)c1cc(cc3ccccc13)CCCCCCNC2] | 401.47 [M + H]⁺_ |
| 6 | 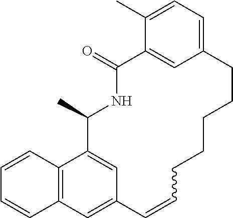 [SMILES: Cc1ccc2cc1C(=O)N[C@H](C)c1cc(cc3ccccc13)C=CCCCCC2] | 384.37 [M + H]⁺ |
| 7 | 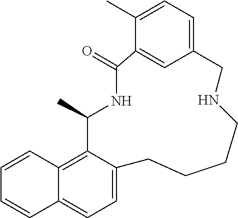 [SMILES: Cc1ccc2cc1C(=O)N[C@H](C)c1c(ccc3ccccc13)CCCCNC2] | 373.41 [M + H]⁺ |
| 8 | 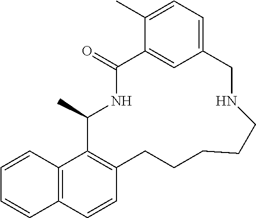 [SMILES: Cc1ccc2cc1C(=O)N[C@H](C)c1c(ccc3ccccc13)CCCCCNC2] | 387.48 [M + H]⁺ |

### Example 9

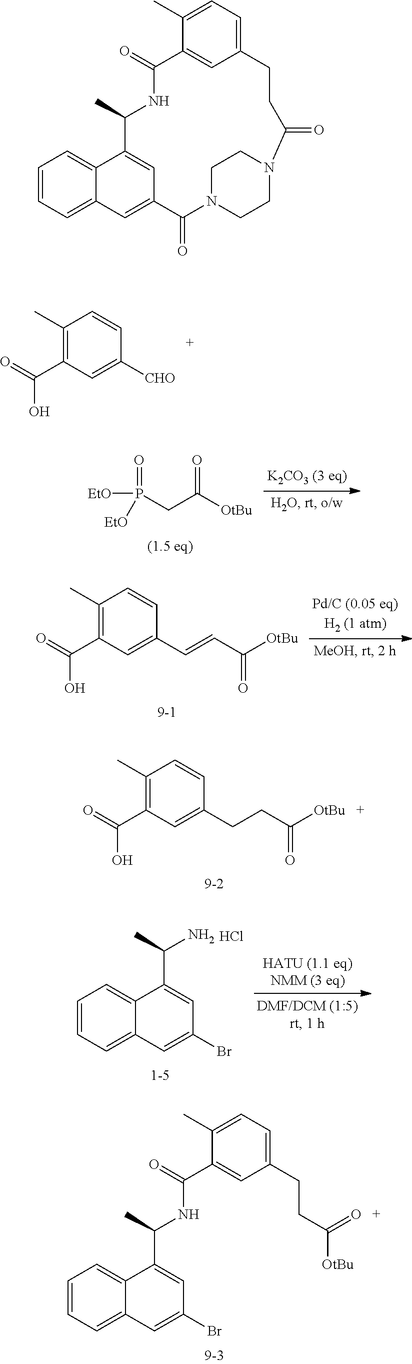

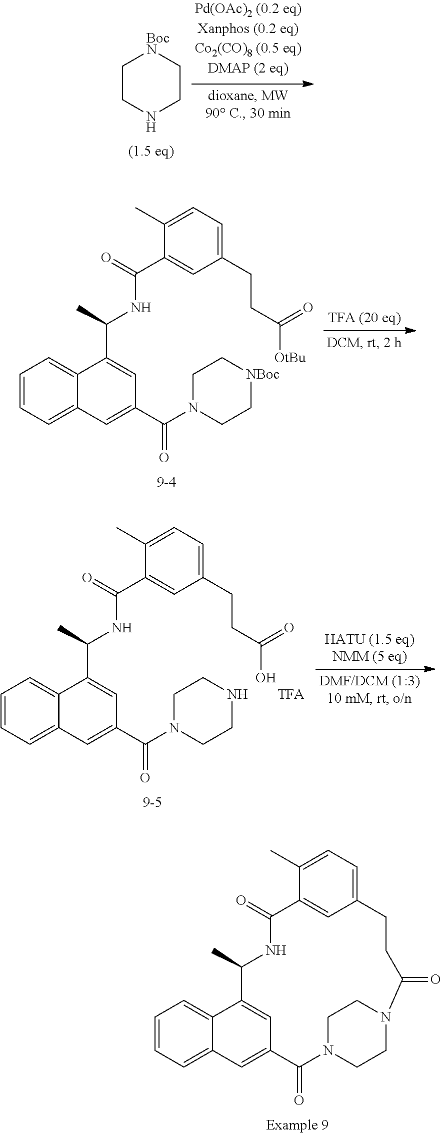 [SMILES: CC(C)(C)OC(=O)N1CCNCC1 ; Cc1ccc(CCC(=O)OC(C)(C)C)cc1C(=O)N[C@H](C)c1cc(C(=O)N2CCN(C(=O)OC(C)(C)C)CC2)cc2ccccc12 ; Cc1ccc(CCC(=O)O)cc1C(=O)N[C@H](C)c1cc(C(=O)N2CCNCC2)cc2ccccc12 ; Cc1ccc2cc1C(=O)N[C@H](C)c1cc(cc3ccccc13)C(=O)N1CCN(CC1)C(=O)CC2]

### Step 9-1:

A solution of 5-formyl-2-methylbenzoic acid (471 mg, 2.87 mmol) in Water (5.5 ml) was treated with K₂CO₃ (1.27 g, 9.19 mmol) and tert-butyl 2-(diethoxyphosphoryl)acetate (850 μl, 3.62 mmol). The reaction was stirred at room temperature over weekend. The mixture was filtered and rinsed with water. The filtrate was dried under high vacuum to give the desired product (633 mg, 2.413 mmol, 84% yield) as a white solid. ESI MS m/z=260.91 [M−H]⁻.

### Step 9-2:

A solution of the compound from Step 9-1 (633 mg, 2.413 mmol) in MeOH (20 ml) was treated with Pd—C (127 mg, 0.119 mmol) under H₂ (1 atm). The reaction was stirred at room temperature for 2 h. The mixture was filtered through celite and concentrated in vacuo to give the desired product (630 mg, 2.383 mmol, 99% yield) as a white solid. ESI MS m/z=262.97 [M−H]⁻.

### Step 9-3:

A suspension of the compound from Step 1-5 (106 mg, 0.370 mmol) and the compound from Step 9-2 (112 mg, 0.424 mmol) in DMF (0.5 ml) and CH₂Cl₂ (2 ml) was treated with HATU (160 mg, 0.421 mmol) and N-methylmorpholine (150 μl, 1.364 mmol). The reaction was stirred at room temperature for 1 h. The reaction was quenched with a saturated solution of sodium bicarbonate. The aqueous layer was extracted with dichloromethane over 3 times. The combined organic layer was dried over sodium sulfate, filtered, and concentrated in vacuo. The crude was added to a 12 g silica gel column and eluted by ethyl acetate/cyclohexane from 0% to 100% to give the desired product (194 mg, 0.391 mmol, 100% yield) as a white solid. ESI MS m/z=518.24, 520.19 [M+H]⁺.

### Step 9-4:

A solution of the compound from Step 9-3 (62 mg, 0.125 mmol) and tert-butyl piperazine-1-carboxylate (36 mg, 0.193 mmol) in dioxane (1 ml) was treated with DMAP (29 mg, 0.237 mmol), xantphos (20 mg, 0.035 mmol), Pd(OAc)₂ (5.8 mg, 0.026 mmol) and CO₂(CO)₈ (25 mg, 0.073 mmol) under N₂. The mixture was bubbled with N2 over 2 min. The reaction was warmed to 90° C. and stirred under microwave irradiation. The mixture was filtered through celite and concentrated in vacuo. The crude was added to a 4 g silica gel column and eluted by ethyl acetate/cyclohexane from 0% to 100% to give the desired product (54 mg, 0.086 mmol, 68.7% yield) as an off-white solid. ESI MS m/z=628.31 [M−H]⁻.

### Step 9-5:

A solution of the compound from Step 9-4 (54 mg, 0.086 mmol) in CH₂Cl₂ (0.6 ml) was treated with TFA (150 μl, 1.947 mmol). The reaction was stirred at room temperature for 2 h. The mixture was concentrated in vacuo to give the desired product (0.086 mmol, 100%) as a white solid. ESI MS m/z=474.08 [M+H]⁺.

### Step 9-6:

A solution of the compound from Step 9-5 (49 mg, 0.086 mmol) in DMF (1 mL) and CH₂Cl₂ (6 ml) was treated with N-methylmorpholine (100 μl, 0.910 mmol) and a solution of HATU (50 g, 131 mmol) in DMF (1 mL) dropwise over 5 min at room temperature. The reaction was stirred at room temperature overnight. The reaction solution was concentrated in vacuo, then diluted with dichloromethane and quenched with a saturated solution of sodium bicarbonate. The aqueous layer was extracted with dichloromethane over 3 times. The combined organic layer was dried over sodium sulfate, filtered and concentrated in vacuo. The crude was added to a 4 g silica gel column and eluted by dichloromethane/methanol from 0% to 20% to give Example 9 (14 mg, 0.031 mmol, 36% yield) as a white solid. ESI MS m/z=456.40 [M+H]⁺.

TABLE 2
The following example was prepared employing similar protocol as described above.

| Example | Structure | MS |
|---|---|---|
| 11 | 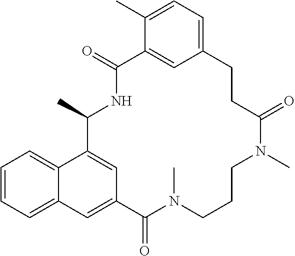 [SMILES: Cc1ccc2cc1C(=O)N[C@H](C)c1cc(cc3ccccc13)C(=O)N(C)CCCN(C)C(=O)CC2] | 470.27 [M − H]⁻ |

### Example 12

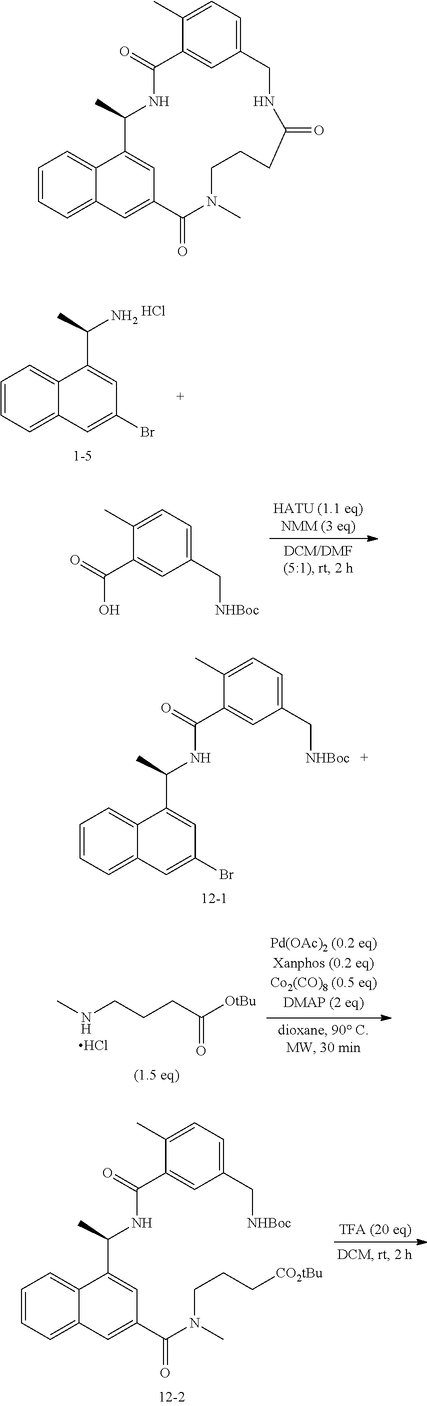

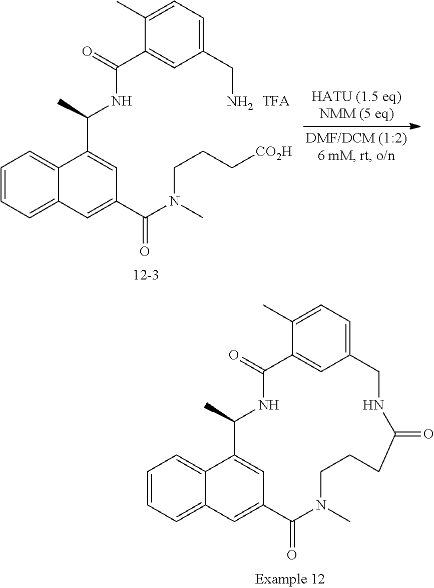 [SMILES: Cc1ccc(CN)cc1C(=O)N[C@H](C)c1cc(C(=O)N(C)CCCC(=O)O)cc2ccccc12 ; Cc1ccc2cc1C(=O)N[C@H](C)c1cc(cc3ccccc13)C(=O)N(C)CCCC(=O)NC2]

### Step 12-1:

A solution of the compound from 1-5 (584 mg, 2.038 mmol) and 5-(((tert-butoxycarbonyl)amino)methyl)-2-methylbenzoic acid (543 mg, 2.047 mmol) in DCM (5 ml) and DMF (1 ml) was treated with N-methylmorpholine (700 μl, 6.37 mmol) and HATU (851 mg, 2.238 mmol). The reaction was stirred at room temperature for 2 h. The mixture was filtered and the solid was rinsed with dichloromethane over 3 times. The filtrate was concentrated. The crude was added to a 24 g silica gel column and eluted by ethyl acetate/cyclohexane from 0% to 100% to give the desired product (0.98 g, 1.970 mmol, 97% yield) as a white solid. ESI MS m/z=495.16, 497.13 [M−H]⁻.

### Step 12-2:

A solution of the compound from Step 12-1 (70 mg, 0.141 mmol) and tert-butyl 4-(methylamino)butanoate hydrochloride (46 mg, 0.219 mmol) in 1,4-Dioxane (1 ml) was treated with Pd(OAc)₂ (6.7 mg, 0.030 mmol), xantphos (18 mg, 0.031 mmol), DMAP (33 mg, 0.270 mmol), TEA (30 μl, 0.215 mmol) and Dicobalt octacarbonyl (24 mg, 0.070 mmol) under N2. The mixture was bubbled with N₂ for 3 min. The reaction was warmed to 90° C. and stirred for 30 min under microwave irradiation. The mixture was filtered through celite and rinsed with acetone. The filtrate was concentrated in vacuo. The crude was added to a 4 g silica gel column and eluted by ethyl acetate/cyclohexane from 0% to 100% to give the desired product (65 mg, 0.105 mmol, 75% yield) as a yellow syrup. ESI MS m/z=640.34 [M+Na]⁺.

### Step 12-3:

A solution of the compound from Step 12-2 (65 mg, 0.105 mmol) in DCM (1 ml) was treated with TFA (0.2 ml, 2.60 mmol). The reaction was stirred at room temperature overnight. The mixture was concentrated in vacuo to give the desired product (59 mg, 0.106 mmol, 100% yield) as a yellow solid. ESI MS m/z=462.38 [M+H]⁺.

### Step 12-4:

A solution of the compound from Step 12-3 (59 mg, 0.106 mmol) in CH₂Cl₂ (10 ml) was treated with N-methylmorpholine (100 μl, 0.910 mmol) and a solution of HATU (65 mg, 0.171 mmol) in DMF (5 ml) dropwise over 5 min. The reaction was stirred at room temperature overnight. The mixture was concentrated in vacuo and diluted with ethyl acetate. The organic layer was washed with brine, dried over sodium sulfate, filtered and concentrated in vacuo. The crude was added to a 4 g silica gel column and eluted by dichloromethane/methanol from 0% to 20% to give Example 12 (18 mg, 0.041 mmol, 38% yield) as an off-white solid. ESI MS m/z=442.23 [M−H]⁻.

TABLE 3
The following examples were prepared employing similar protocol as described above.
Triazole is introduced via CuAAc. Piperazine is introduced via Buchwald-Hartwig coupling.

| Example | Structure | MS |
|---|---|---|
| 13 | 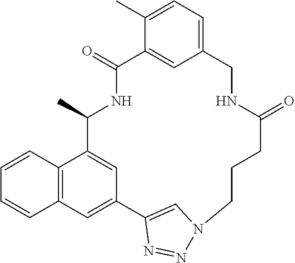 [SMILES: Cc1ccc2cc1C(=O)N[C@H](C)c1cc(cc3ccccc13)-c1cn(nn1)CCCC(=O)NC2] | 454.28 [M + H]⁺ |
| 14 | 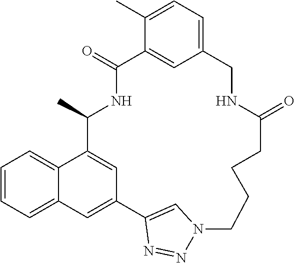 [SMILES: Cc1ccc2cc1C(=O)N[C@H](C)c1cc(cc3ccccc13)-c1cn(nn1)CCCCC(=O)NC2] | 468.37 [M + H]⁺ |
| 15 | 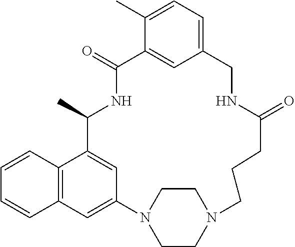 [SMILES: Cc1ccc2cc1C(=O)N[C@H](C)c1cc(cc3ccccc13)N1CCN(CCCC(=O)NC2)CC1] | 471.78 [M + H]⁺ |
| 16 | 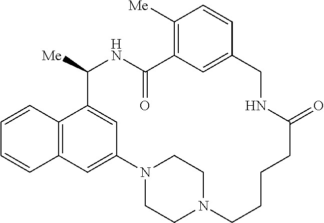 [SMILES: Cc1ccc2cc1C(=O)N[C@H](C)c1cc(cc3ccccc13)N1CCN(CCCCC(=O)NC2)CC1] | 485.42 [M + H]⁺ |

### Example 17

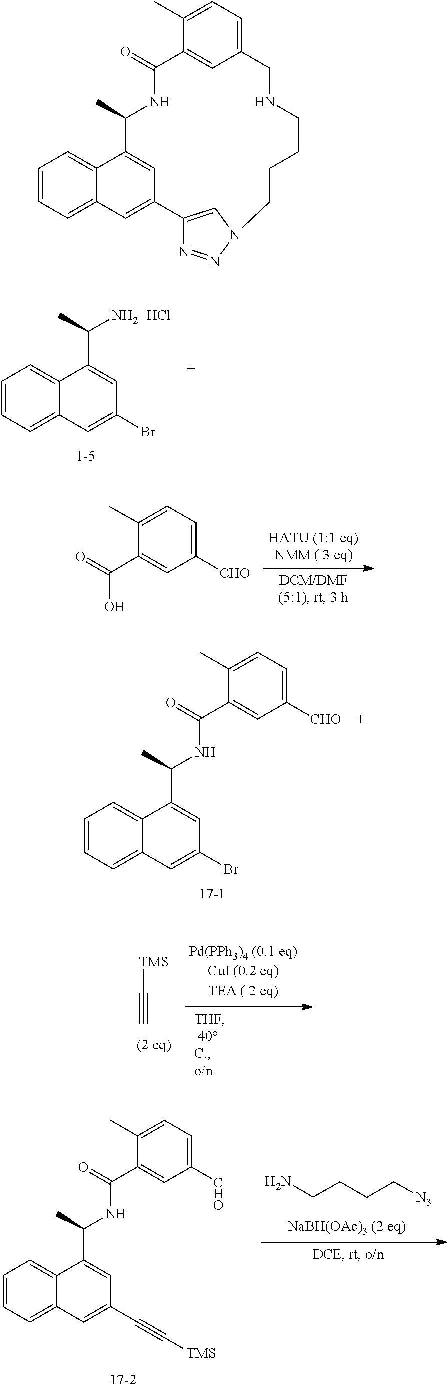

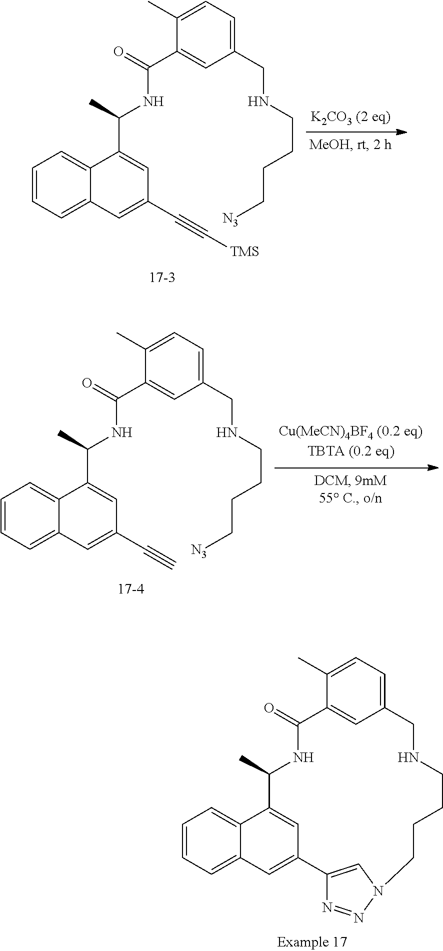 [SMILES: Cc1ccc(CNCCCCN=[N+]=[N-])cc1C(=O)N[C@H](C)c1cc(C#C[Si](C)(C)C)cc2ccccc12 ; C#Cc1cc([C@@H](C)NC(=O)c2cc(CNCCCCN=[N+]=[N-])ccc2C)c2ccccc2c1 ; Cc1ccc2cc1C(=O)N[C@H](C)c1cc(cc3ccccc13)-c1cn(nn1)CCCCNC2]

### Step 17-1:

A solution of the compound from Step 1-5 (366 mg, 1.277 mmol) and 5-formyl-2-methylbenzoic acid (219 mg, 1.334 mmol) in DCM (4 ml) and DMF (0.5 ml) was treated with N-methylmorpholine (450 μl, 4.09 mmol) and HATU (539 mg, 1.418 mmol). The reaction was stirred at room temperature for 3 h and then quenched with a saturated solution of sodium bicarbonate. The aqueous layer was extracted with dichloromethane over 3 times. The combined organic layer was dried over sodium sulfate, filtered and concentrated in vacuo. The crude was added to a 40 g silica gel column and eluted by ethyl acetate/cyclohexane from 0% to 100% to give the desired product (1 mmol, 78%) as a white solid. ESI MS m/z=395.72, 397.69 [M+H]⁺.

### Step 17-2:

A solution of the compound from Step 17-1 (99 mg, 0.250 mmol) in THF (2 ml) was treated with ethynyltrimethylsilane (80 μl, 0.562 mmol), TEA (110 μl, 0.789 mmol), copper(I) iodide (12 mg, 0.063 mmol) and Pd(Ph₃P)₄ (30 mg, 0.026 mmol) under N₂. The mixture was bubbled with N₂ over 3 min. The reaction was warmed to 40° C. and stirred overnight. The mixture was filtered through celite and rinsed with acetone. The filtrate was concentrated in vacuo. The crude was added to a 4 g silica gel column and eluted by ethyl acetate/cyclohexane from 0% to 100% to give the desired product (92 mg, 0.222 mmol, 89% yield) as a white solid. ESI MS m/z=412.41 [M−H]⁻.

### Step 17-3:

A solution of the compound from Step 17-2 (92 mg, 0.222 mmol) and 4-azidobutan-1-amine (55 mg, 0.482 mmol) in DCE (2 ml) was treated with sodium triacetoxyborohydride (94 mg, 0.444 mmol). The reaction was stirred at room temperature overnight and then quenched with a saturated solution of sodium bicarbonate. The aqueous layer was extracted with dichloromethane over 3 times. The combined organic layer was dried over sodium sulfate, filtered and concentrated in vacuo. The crude was added to a 4 g silica gel column and eluted by dichloromethane/methanol from 0% to 20% to give the desired product (69 mg, 0.135 mmol, 61% yield) as a white solid. ESI MS m/z=512.58 [M+H]⁺.

### Step 17-4:

A solution of the compound from Step 17-3 (69 mg, 0.135 mmol) in MeOH (2 ml) was treated with K₂CO₃ (33 mg, 0.239 mmol). The reaction was stirred at room temperature for 2 h. The mixture was concentrated, diluted with dichloromethane and filtered to give the desired product (57 mg, 0.130 mmol, 96% yield) as a yellow syrup. ESI MS m/z=440.43 [M+H]⁺.

### Step 17-5:

A solution of the compound from Step 17-4 (57 mg, 0.130 mmol) in CH₂Cl₂ (15 ml) was treated with Cu(MeCN)₄BF₄ (9 mg, 0.029 mmol) and TBTA (13 mg, 0.024 mmol). The reaction was warmed to 55° C. and stirred overnight. The mixture was filtered through celite and washed with water and dichloromethane to give Example 17 (43 mg, 0.098 mmol, 75% yield) as a light green solid. ESI MS m/z=440.57 [M+H]⁺.

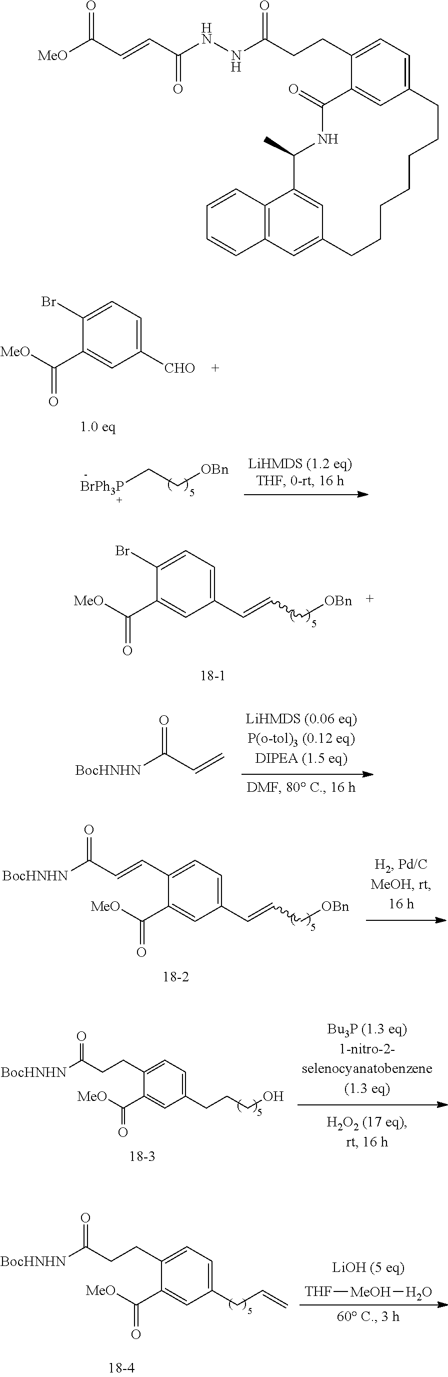

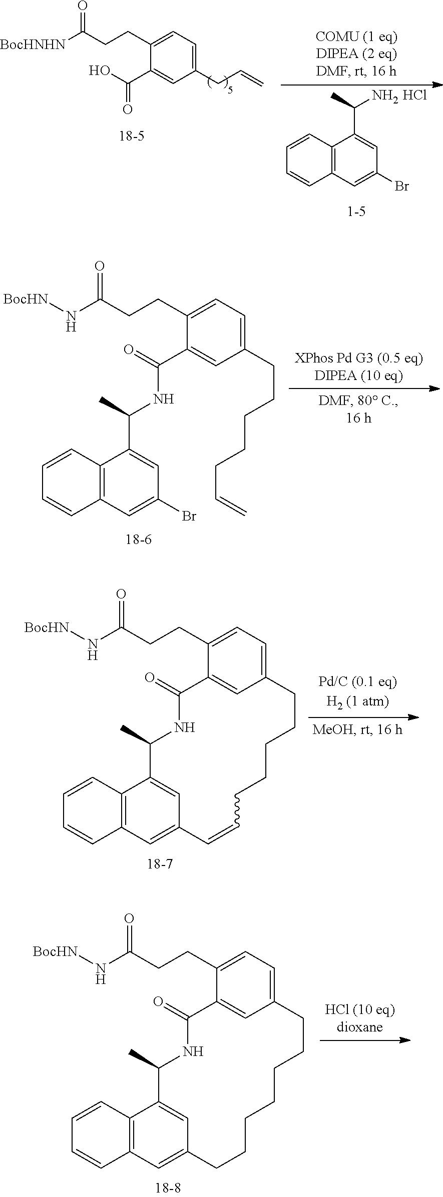

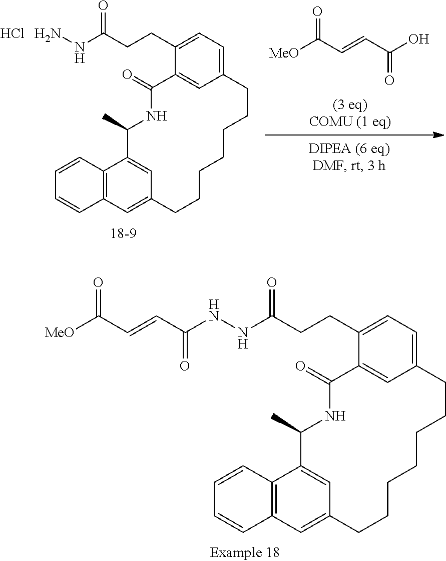 [SMILES: Cl ; C[C@H]1NC(=O)c2cc(ccc2CCC(=O)NN)CCCCCCCc2cc1c1ccccc1c2 ; COC(=O)/C=C/C(=O)O ; COC(=O)/C=C/C(=O)NNC(=O)CCc1ccc2cc1C(=O)N[C@H](C)c1cc(cc3ccccc13)CCCCCCC2]

### Example 18

### Step 18-1:

To a solution of (6-(benzyloxy)hexyl)triphenylphosphonium bromide (3.87 g, 7.25 mmol) in THF (20 mL) at 0° C. was treated with LiHMDS (6.70 ml, 6.70 mmol). After 30 mins, methyl 2-bromo-5-formylbenzoate (1.356 g, 5.58 mmol) in THF (5 mL) was added slowly to it and stirred at for 3 h. It was quenched with aq NH₄Cl, extracted with MBTE, washed with water, brine, dried over Na₂SO₄, filtered, conc. And purified by silica gel column to give the desired product (1.48 g, 3.55 mmol, 64% yield). ESI MS m/z=439.09, 441.09 [M+Na]⁺.

### Step 18-2:

A mixture of the compound from Step 18-2 (1.1 g, 2.64 mmol), palladium (II) aceate (0.036 g, 0.158 mmol), tri-o-tolylphosphine (0.096 g, 0.316 mmol), DIPEA (0.691 ml, 3.95 mmol) and tert-butyl 2-acryloylhydrazine-1-carboxylate (0.589 g, 3.16 mmol) in DMF (3.0 mL) was stirred at 80° C. for 16 h. It was diluted with EtOAc, filtered through celite, washed with water, brine, dried over Na₂SO₄, filtered, conc. And purified by silica gel column to give the desired product (0.9 g, 1.722 mmol, 65% yield). ESI MS m/z=545.26 [M+Na]⁺.

### Step 18-3:

A mixture of the compound from Step 18-2 (0.9 g, 1.722 mmol) and 10% Pd—C (0.367 g, 0.344 mmol) in MeOH (10 mL) was treated with a hydrogen balloon for 20 h. It was filtered through celite, conc. And purified by silica gel column to give the desired product (0.59 g, 1.352 mmol, 78% yield). ESI MS m/z=459.25 [M+Na]⁺.

### Step 18-4:

To a solution of the compound from Step 18-3 (0.59 g, 1.352 mmol) and 1-nitro-2-selenocyanatobenzene (0.399 g, 1.757 mmol) in THF (10 mL) was added tri-n-butylphosphine (0.434 ml, 1.757 mmol) and stirred at rt for 1.5 h. Hydrogen peroxide (2.347 ml, 22.98 mmol) was added to the mixture and stirred at rt for 18 h. The mixture was treated with sat NaHCO₃, extracted with EtOAc, washed with Na₂S₂O₃, brine, dried over Na₂SO₄, filtered, conc., and purified by silica gel column to the desired product (0.44 g, 1.051 mmol, 78% yield). ESI MS m/z=441.23 [M+Na]⁺.

### Step 18-5:

To a solution of the compound from Step 18-4 (440 mg, 1.051 mmol) in THF-water (4/2 mL) was added LiOH (151 mg, 6.31 mmol) and stirred at 60 for 2 h. it was ocnc, diluted with water, acidified with 1N HCl, extracted with EtOAc, washed with water, brine, dry over Na₂SO₄, filtered, conc to give the desired product (0.42 g, 1.038 mmol, 99% yield).

### Step 18-6:

To a solution of the compound from Step 18-5 (425 mg, 1.051 mmol), €-1-(3-bromonaphthalen-1-yl)ethan-1-amine-methane (1/1) hydrochloride (318 mg, 1.051 mmol) and DIPEA (551 μl, 3.15 mmol) in DMF (5 mL) was added COMU (450 mg, 1.051 mmol), after 2 h at rt, it was diluted with EtOAc, washed with 1N HCl, sat NaHCO₃, brine to give the desired product (350 mg, 0.550 mmol, 52.3% yield). ESI MS m/z=636.22, 638.24 [M+H]⁺.

### Step 18-7:

A solution of Xphos Pd G3 (23.27 mg, 0.027 mmol), the compound from Step 18-6 (35 mg, 0.055 mmol) and DIPEA (96 μl, 0.550 mmol) in DMF (4 mL), after 80 for 16 h, it was diluted with EtOAc, filtered through celite, washed with water, brine, dry over Na₂SO₄, filtered, conc column to give the desired product (12 mg, 0.022 mmol, 39% yield).

### Step 18-8:

A mixture of the compound from Step 18-7 (18 mg, 0.032 mmol) and Pd—C (6.89 mg, 6.48 μmol) in MeOH (3 mL) was treated with H₂, for 16 h. The mixture was filtered to give the desired product (18 mg, 0,032 mmol, 100% yield).

### Step 18-9:

A mixture of the compound from Step 18-8 (18 mg, 0.032 mmol) and MeOH (0.3 mL), DCM (1.5 mL) was treated with 4N HCl (2 mL) and stirred at rt for 3 h. The mixture was concentrated to give the desired product (16 mg, 0.032 mmol, 100% yield)

### Step 18-10:

A mixture of the compound from Step 18-9 (0.016 g, 0.032 mmol), €-4-methoxy-4-oxobut-2-enoic acid (9.16 mg, 0.070 mmol), DIPEA (0.028 ml, 0.160 mmol) and COMU (0.016 g, 0.038 mmol) in DMF (1 mL) was stirred at rt for 3 h. The mixture was purified by HPLC to give Example 18 (4.2 mg, 0.0074 mmol, 23% yield) as a white solid. ESI MS m/z=570.34 [M+H]⁺.

TABLE 4
The following examples were prepared employing similar protocol as described above.

| Example | Structure | MS |
|---|---|---|
| 19 | 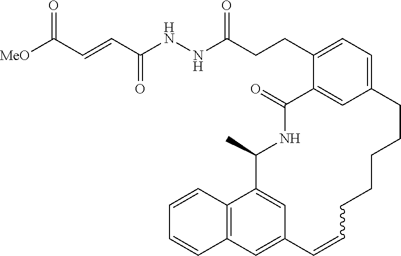 [SMILES: COC(=O)/C=C/C(=O)NNC(=O)CCc1ccc2cc1C(=O)N[C@H](C)c1cc(cc3ccccc13)C=CCCCCC2] | 568.29 [M + H]⁺ |
| 20 | 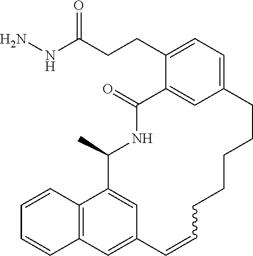 [SMILES: C[C@H]1NC(=O)c2cc(ccc2CCC(=O)NN)CCCCCC=Cc2cc1c1ccccc1c2] | 456.34 [M + H]⁺ |
| 21 | 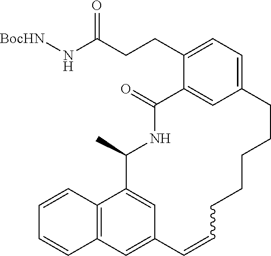 [SMILES: C[C@H]1NC(=O)c2cc(ccc2CCC(=O)NNC(=O)OC(C)(C)C)CCCCCC=Cc2cc1c1ccccc1c2] | 556.43 [M + H]⁺ |
| 22 | 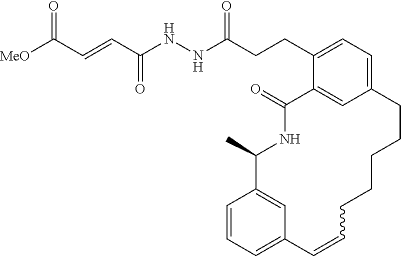 [SMILES: COC(=O)/C=C/C(=O)NNC(=O)CCc1ccc2cc1C(=O)N[C@H](C)c1cccc(c1)C=CCCCCC2] | 518.39 [M + H]⁺ |

### Example 23

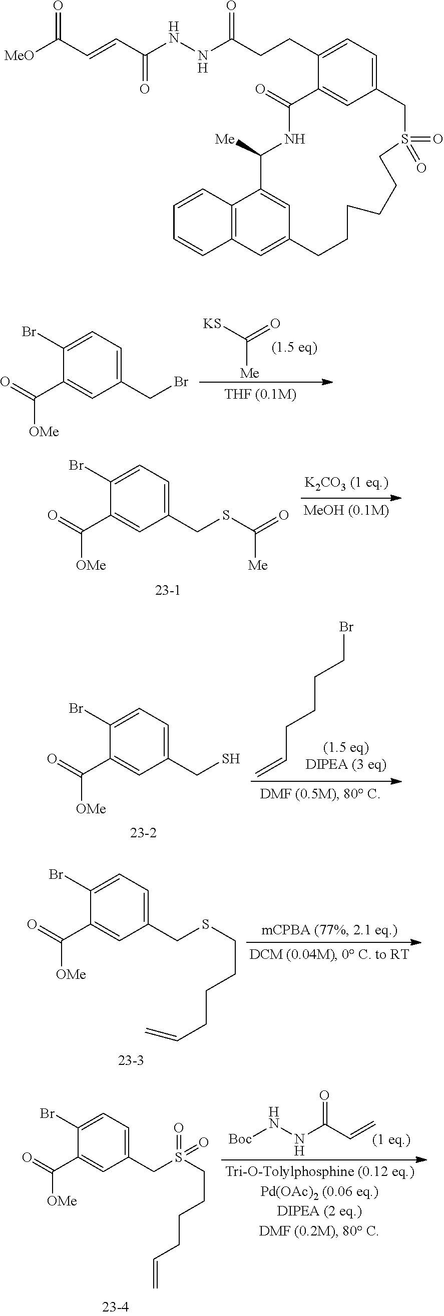

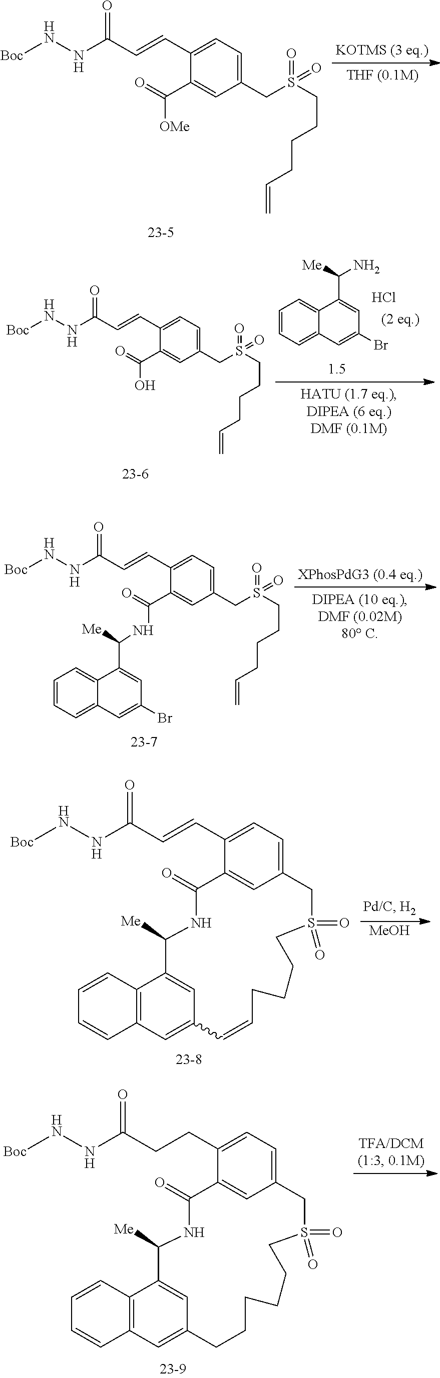

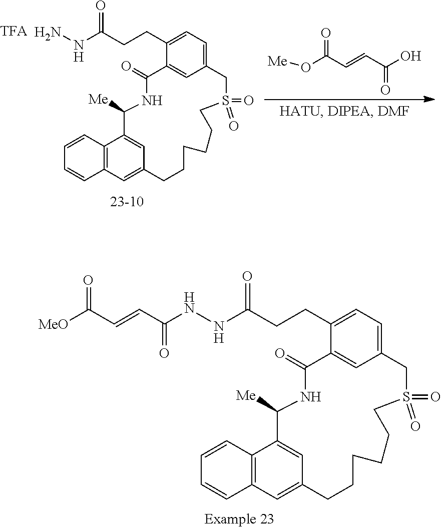 [SMILES: C[C@H]1NC(=O)c2cc(ccc2CCC(=O)NN)CS(=O)(=O)CCCCCCc2cc1c1ccccc1c2 ; COC(=O)/C=C/C(=O)O ; COC(=O)/C=C/C(=O)NNC(=O)CCc1ccc2cc1C(=O)N[C@H](C)c1cc(cc3ccccc13)CCCCCCS(=O)(=O)C2]

### Step 23-1:

Acetylsulfanylpotassium (1.50 eq, 556 mg, 4.87 mmol) and methyl 2-bromo-5-(bromomethyl)benzoate (1.00 eq, 1.00 g, 3.25 mmol) were added to a RB flask. The reaction flask was purged with nitrogen. THF (32.471 mL) was then added. The reaction mixture was allowed to stir at room temperature for 16 hours. After the completion of the reaction, the mixture was concentrated and subjected to Combiflash (cycHex:EA 100:0->0:100) and the desired product was isolated in 88% yield. ESI MS m/z=304, 305 [M+H]⁺.

### Step 23-2:

To the solution of the compound from Step 23-2 (1.00 eq, 670 mg, 2.21 mmol) in Methanol (20 mL) was added K₂CO₃ (1.00 eq, 305 mg, 2.21 mmol). The reaction was then allowed to stir at room temperature for 2.5 hours. 1N HCl was added to quench the reaction and pH was checked with pH paper to confirm pH reached 2-3. The mixture was then diluted with water and DCM. The aqueous layer was extracted with DCM 3 times (30 mL). The organic layers were then combined, dried and concentrated. The material was then subjected to column (DCM:MeOH 0 to 20%) to afford desired product in 88% yield. ESI MS m/z=262, 263 [M+H]⁺.

### Step 23-3:

The compound from Step 23-2 (1.00 eq, 200 mg, 0.766 mmol) was added to a seal tube. The tube was purged with nitrogen three times. 6-BROMO-1-HEXENE (1.50 eq, 0.15 mL, 1.15 mmol) and N,N-Diisopropylethylamine (3.00 eq, 0.40 mL, 2.30 mmol) were added to the tube. Finally, DMF (1.5318 mL) was added. The reaction mixture was then heated to 80 degrees, and it was allowed to stir at this temperature overnight under N₂. After the completion of the reaction, the mixture was diluted with 1 mL of water and extracted with 20 mL of ethyl acetate. The organic layer was dried and concentrated. The desired product was obtained after purification of the concentrated material with CombiFlash (cycHex:EA 100:0->0:100) in 97% yield. ESI MS m/z=345, 346 [M+H]⁺.

### Step 23-4:

To a solution of the compound from Step 23-3 (1.00 eq, 266 mg, 0.775 mmol) in DCM (19.372 mL) was added 3-CHLOROPEROXYBENZOIC ACID (2.10 eq, 365 mg, 1.63 mmol) at 0 degree. The reaction mixture was then warmed up to room temperature. The reaction was then quenched with Na₂SO₃ 204 mg (1.63 mmol) and diluted with water (2 mL). The reaction mixture was then extracted with EA (3×20 mL). The organic layers were then combined and dried. After the organic layers were concentrated, the material was subjected to Combiflash (cycHex:EA 100:0->0:100) to obtain the desired product in 86% yield. ESI MS m/z=376, 377 [M+H]⁺.

### Step 23-5:

The compound from Step 23-4 (1.00 eq, 250 mg, 0.666 mmol), PALLADIUM(II) ACETATE (0.0600 eq, 9.0 mg, 0.0400 mmol) and TRI-O-TOLYLPHOSPHINE (0.124 eq, 25 mg, 0.0828 mmol) were added to a sealed vial. The vial was then purged with nitrogen for 3 times to make sure the atmosphere is all inert gas. DMF (3.3308 mL) and N,N-Diisopropylethylamine (2.00 eq, 0.23 mL, 1.33 mmol) added in sequence. The reaction mixture was then heated to 80 degrees, and the vial was still connected to the gas line. After 3 hours, the reaction was stopped by cooling it to room temperature and concentrated. The mixture was then subjected to Combi Flash (DCM:MeOH 100:0->80:20), and the desired product was obtained in 77% yield. ESI MS m/z=481 [M+H]⁺.

### Step 23-6:

To a solution of the compound from Step 23-5 (1.00 eq, 340 mg, 0.707 mmol) in THF (7.0748 mL) was added trimethylsilyloxypotassium (3.00 eq, 272 mg, 2.12 mmol) at 0 degree. The reaction mixture was warmed to room temperature. Citric acid (400 mg, 2.12 mmol) was added to quench the reaction after 2 hours. The reaction was then diluted with water (3 mL) and extracted with EA (3×10 mL). The organic layers were combined and dried. The crude material was used directly in the next step after concentration. ESI MS m/z=467 [M+H]⁺.

### Step 23-7:

To a solution of the compound from Step 23-6 (1.00 eq, 364 mg, 0.780 mmol) and the compound from Step 1-5 (1.50 eq, 335 mg, 1.17 mmol) in DMF (7.802 mL) were added N,N-Diisopropylethylamine (6.00 eq, 0.82 mL, 4.68 mmol) and HATU (1.50 eq, 445 mg, 1.17 mmol). The reaction mixture was allowed to stir at room temperature for 2 hours. The mixture was diluted with water (3 mL) and extracted with EA (30 mL). The organic lay was dried and concentrated. The material was then subjected to CombiFlash (cycHex:EA 100:0->0:100) to afford the desired product in 52% yield. ESI MS m/z=698, 700 [M+H]⁺.

### Step 23-8:

To a solution the compound from Step 23-7 (1.00 eq, 285 mg, 0.408 mmol) and Xphos Pd G3 (0.400 eq, 138 mg, 0.163 mmol) in DMF (20.396 mL) was added N,N-Diisopropylethylamine (10.0 eq, 0.71 mL, 4.08 mmol). The reaction mixture was allowed to stir at 80 degrees for 6 hours. The- reaction was then quenched with water (20 mL) and extracted with EA (100 mL). The organic layer was then dried and concentrated. The material was subsequently subjected to CombiFlash (DCM:MeOH 100:0→80:20) to give the desired product in 69% yield. ESI MS m/z=618 [M+H]⁺.

### Step 23-9:

The compound from Step 23-8 (1.00 eq, 174 mg, 0.282 mmol) and Pd/C (34 mg, 20% W) were added to a vial. Methanol (14.083 mL). The reaction vessel was purged with hydrogen three times. The reaction mixture was allowed to stir at room temperature for 3 hours. The reaction mixture was then filtered through a pad of Celite. The filtrate was concentrated and used in the next step directly. ESI MS m/z=622 [M+H]⁺.

### Step 23-10:

To the solution of the compound from Step 23-10 (1.00 eq, 100 mg, 0.161 mmol) in DCM (3.2375 mL) was added TFA (1.0 mL). The reaction mixture was allowed to stir at room temperature for 1 hour before being concentrated for the next step. ESI MS m/z=522 [M+H]⁺.

### Step 23-11:

To the solution of €-4-methoxy-4-oxo-but-2-enoic acid (1.30 eq, 27 mg, 0.209 mmol), the compound from Step 23-10 (1.00 eq, 84 mg, 0.161 mmol) and N,N-Diisopropylethylamine (6.00 eq, 0.17 mL, 0.966 mmol) in DMF (1.6102 mL) was added HATU (1.30 eq, 80 mg, 0.209 mmol). The reaction mixture was allowed to stir at room temperature for 1 hour. The reaction was quenched by adding water and the mixture was extracted with EA (3×20 mL). The organic layers were combined and dried, and the material was subjected to Combiflash (DCM:MeOH 100:0->80:20) to afford the Example 23 as an off-white solid (15 mg, 0.0237 mmol, 14.70% yield). ESI MS m/z=634.06 [M+H]⁺.

TABLE 5
The following examples were prepared employing similar protocol as described above.

| Example | Structure | MS |
|---|---|---|
| 24 | 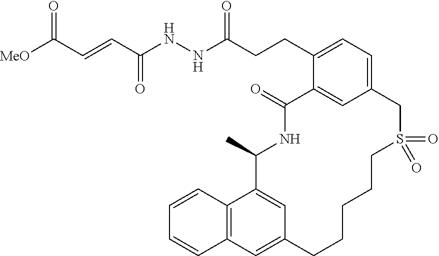 [SMILES: COC(=O)/C=C/C(=O)NNC(=O)CCc1ccc2cc1C(=O)N[C@H](C)c1cc(cc3ccccc13)CCCCCS(=O)(=O)C2] | 618.57 [M − H]⁻ |

### Example 25

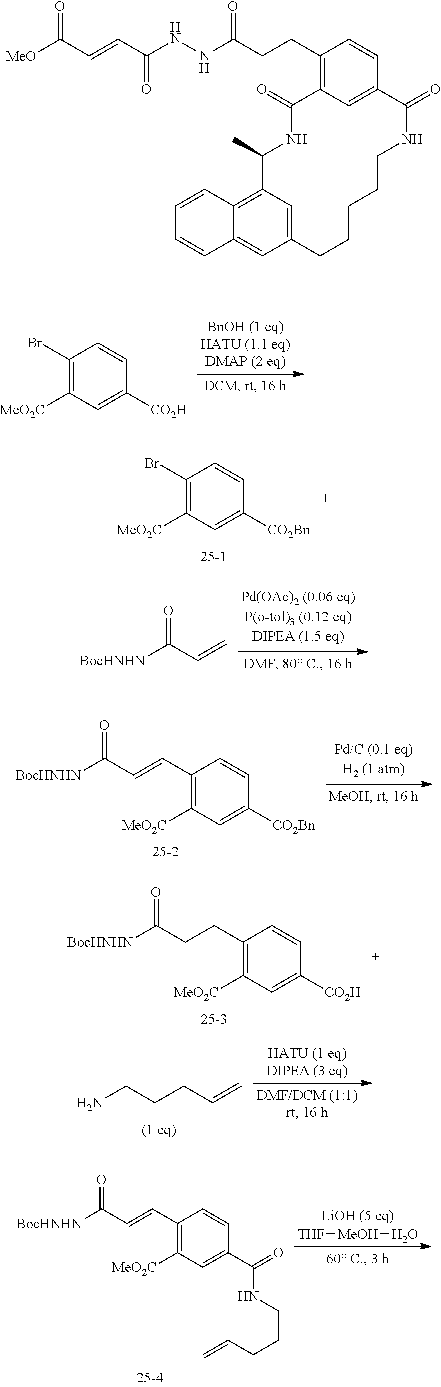

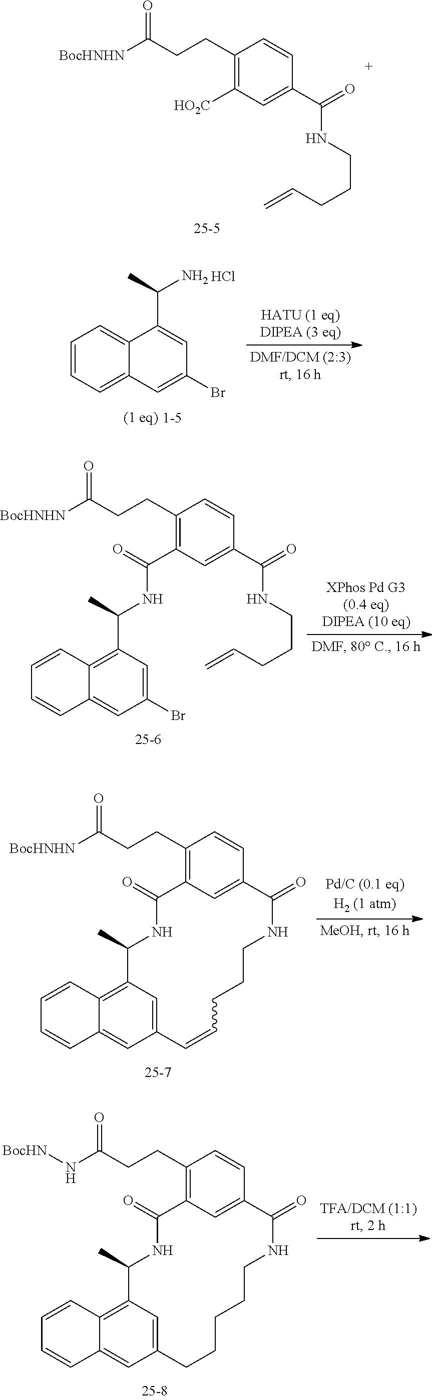

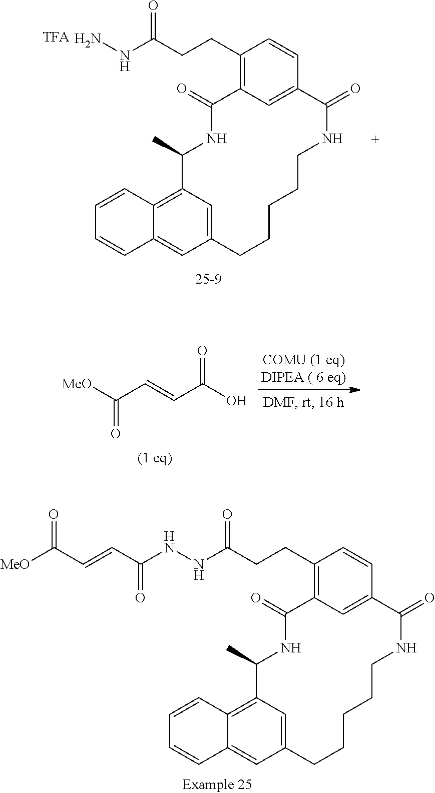 [SMILES: C[C@H]1NC(=O)c2cc(ccc2CCC(=O)NN)C(=O)NCCCCCc2cc1c1ccccc1c2 ; "+" ; COC(=O)/C=C/C(=O)O ; COC(=O)/C=C/C(=O)NNC(=O)CCc1ccc2cc1C(=O)N[C@H](C)c1cc(cc3ccccc13)CCCCCNC2=O]

### Step 25-1:

A mixture of 4-bromo-3-(methoxycarbonyl)benzoic acid (5 g, 19.30 mmol), benzyl alcohol (2.007 ml, 19.30 mmol), DMAP (5.19 g, 42.5 mmol) and HATU (8.81 g, 23.16 mmol) in DCM (38 mL) was stirred at rt for 16 h. The resulted slurry was diluted with DCM, washed with water, brine, dry over Na₂SO₄, filtered, conc, purified by slica gel column to give 1-benzyl 3-methyl 4-bromoisophthalate (6.38 g, 18.27 mmol, 95% yield). ¹H NMR (400 MHz, CDCl₃) δ 8.43 (d, J=2.2 Hz, 1H), 7.97 (dd, J=8.4, 2.2 Hz, 1H), 7.72 (d, J=8.4 Hz, 1H), 7.46-7.29 (m, 5H), 5.35 (s, 2H), 3.93 (s, 3H).

### Step 25-2:

A mixture of tert-butyl 2-acryloylhydrazine-1-carboxylate (1.227 g, 6.59 mmol), the compound from Step 25-1 (2 g, 5.73 mmol), Pd(Oac)₂ (0.077 g, 0.344 mmol), DIPEA (1.501 ml, 8.59 mmol) and tri-o-tolylphosphane (0.209 g, 0.687 mmol) in DMF (7 ml) was stirred at 80° C. for 16 h, cooled to rt, diluted with EtOAc, washed with water, brine, dried over Na₂SO₄, filtered, concentrated, purified by silica gel column to give the desired product (2.44 g, 5.37 mmol, 94% yield) as a white solid. ESI MS m/z=355.13 [M+H-Boc]⁺.

### Step 25-3:

A suspension of the compound from step 25-2 (2.44 g, 5.37 mmol) and Pd—C (5%) (0.571 g, 0.268 mmol) in MeOH (15 mL) was stirred under H₂ Balloon for 16 h. The mixture was filtered through celite, concentrated and dired under 73 acuum to give the desired product (1.9 g, 5.19 mmol, 97% yield). ESI MS m/z=365.14 [M−H]⁻. ¹H NMR (400 MHz, CD₃OD_SPE) δ 8.52 (d, J=1.9 Hz, 1H), 8.09 (dd, J=8.0, 1.9 Hz, 1H), 7.48 (d, J=8.1 Hz, 1H), 3.93 (s, 3H), 3.35-3.32 (m, 2H), 2.56 (t, J=7.8 Hz, 2H), 1.46 (s, 9H).

### Step 25-4:

A mixture of the compound from Step 25-3 (0.55 g, 1.501 mmol), DIPEA (0.655 ml, 3.75 mmol), pent-4-en-1-amine (0.165 ml, 1.501 mmol) and HATU (0.571 g, 1.501 mmol) in DCM/DMF (3/3 mL) was stirred at rt for 16 h. It was diluted with EtOAc, washed with water, brine, dried ovr Na₂SO₄, filtered, conc. and purified by silica gel column to give the desired product (0.6 g, 1.384 mmol, 92% yield). ESI MS m/z=434.23 [M+H]⁺.

### Step 25-5:

A mixture of the compound from Step 25-4 (0.62 g, 1.433 mmol) and LiOH (0.172 g, 7.17 mmol) in THF-MeOH-water (3/2/1 mL) was stirred at 60° C. for 3 h. It was conc, teated with 1N HCl to pH 3, extracted with EtOAc, washed with brine, dried over Na₂SO₄, filtered, conc. To give the desired product (0.58 g, 1.386 mmol, 97% yield). ESI MS m/z=441.24 [M+Na]⁺.

### Step 25-6:

A mixture of the compound from Step 25-5 (0.58 g, 1.386 mmol), the compound from Step 1-5 (0.419 g, 1.386 mmol) and DIPEA (0.847 ml, 4.85 mmol) in DCM-DMF (3/2 mL) at 0° C. was treated with HATU (0.527 g, 1.386 mmol), after 16 h, it was diluted with EtOAc, washed with water, brine, dried over Na₂SO₄, filtered, conc. And purified by silica gel column to give the desired product (0.6 g, 0.922 mmol, 67% yield). ESI MS m/z=650.26, 652.26 [M+H]⁺.

### Step 25-7:

A mixture of the compound from Step 25-6 (300 mg, 0.460 mmol), Xphos Pd G3 (156 mg, 0.184 mmol) and DIPEA (804 μl, 4.60 mmol) in DMF (24 mL) was stirred at 80° C. for 16 h. It was cooled to rt, diluted with EtOAc, filtered through celite, washed with EtOAc, the residue was washed with water, brine, dried over Na₂SO₄, filtered, conc. And purified by silica gel column to give the desired product (136 mg, 0.238 mmol, 52% yield). ESI MS m/z=571.29 [M+H]⁺.

### Step 25-8:

A suspension of the compound from Step 25-7 (80 mg, 0.140 mmol) and 10% Pd—C (29.8 mg, 0.028 mmol) in MeOH (5 mL) was stirred with a H₂ ballon for 16 h. It was filtered through celite, conc. To give the desired product (80 mg, 0.140 mmol, 100% yield). ESI MS m/z=573.31 [M+H]⁺.

### Step 25-9:

A mixture of the compound from Step 25-8 (62 mg, 0.108 mmol) in DCM/TFA (1/1 mL) was stirred at rt for 2 h. It was conc give crude product used in next step directly. ESI MS m/z=473.26 [M+H]⁺.

### Step 25-10:

A mixture of the compound from Step 25-9 (0.065 g, 0.11 mmol), €-4-methoxy-4-oxobut-2-enoic acid (0.014 g, 0.110 mmol), DIPEA (0.067 ml, 0.385 mmol) and COMU (0.047 g, 0.110 mmol) in DMF (1 mL) was stirred at rt for 16 h. It was diluted with DMSO (2 mL) and purified by prep-HPLC to give Example 25 (19 mg, 0.032 mmol, 29.5% yield). ESI MS m/z=585.27 [M+H]⁺.

TABLE 6
The following examples were prepared employing similar protocol as described above.

| Example | Structure | MS |
|---|---|---|
| 26 | 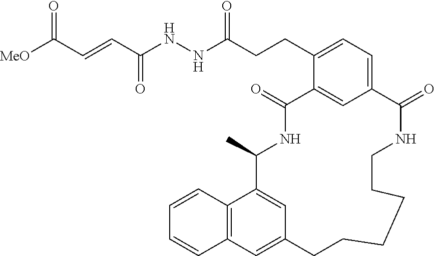 [SMILES: COC(=O)/C=C/C(=O)NNC(=O)CCc1ccc2cc1C(=O)N[C@H](C)c1cc(cc3ccccc13)CCCCCCNC2=O] | 599.28 [M + H]⁺ |
| 27 | 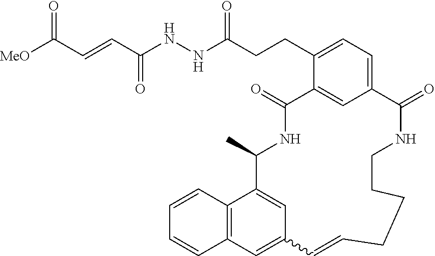 [SMILES: COC(=O)/C=C/C(=O)NNC(=O)CCc1ccc2cc1C(=O)N[C@H](C)c1cc(cc3ccccc13)C=CCCCCNC2=O] | 597.27 [M + H]⁺ |
| 28 | 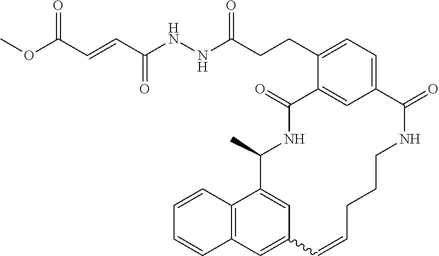 [SMILES: COC(=O)/C=C/C(=O)NNC(=O)CCc1ccc2cc1C(=O)N[C@H](C)c1cc(cc3ccccc13)C=CCCCNC2=O] | 583.25 [M + H]⁺ |
| 29 | 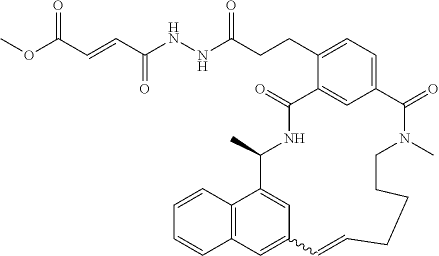 | 611.23 [M + H]⁺ |
| 30 | 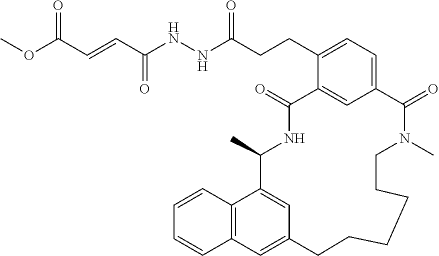 | 613.29 [M + H]⁺ |
| 31 | 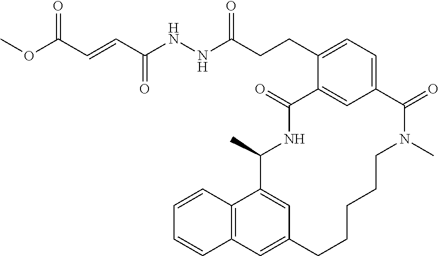 | 599.29 [M + H]⁺ |
| 32 | 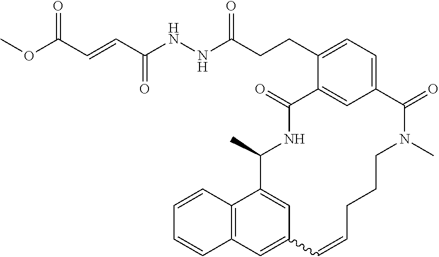 | 597.24 [M + H]⁺ |
| 33 | 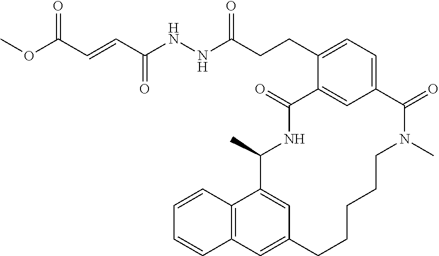 | 603.32 [M + H]⁺ |
| 34 | 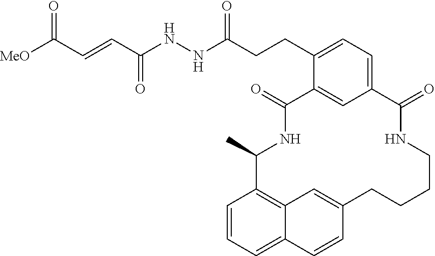 [SMILES: COC(=O)/C=C/C(=O)NNC(=O)CCc1ccc2cc1C(=O)N[C@H](C)c1cccc3ccc(cc13)CCCCNC2=O] | 569.10 [M − H]⁻ |
| 35 | 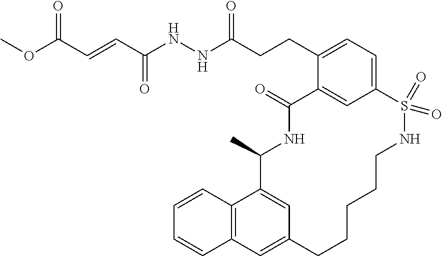 [SMILES: COC(=O)/C=C/C(=O)NNC(=O)CCc1ccc2cc1C(=O)N[C@H](C)c1cc(cc3ccccc13)CCCCCNS2(=O)=O] | 619.228 [M − H]⁻ |
| 36 | 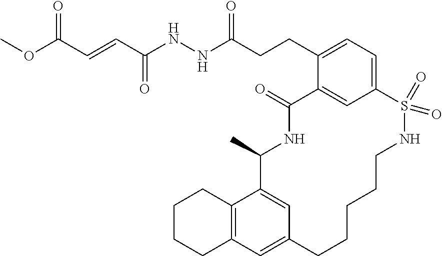 [SMILES: COC(=O)/C=C/C(=O)NNC(=O)CCc1ccc2cc1C(=O)N[C@H](C)c1cc(cc3c1CCCC3)CCCCCNS2(=O)=O] | 623.252 [M − H]⁻ |
| 37 | 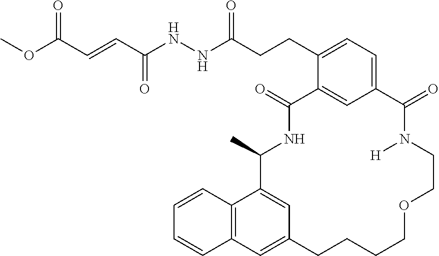 [SMILES: COC(=O)/C=C/C(=O)NNC(=O)CCc1ccc2cc1C(=O)N[C@H](C)c1cc(cc3ccccc13)CCCCOCCNC2=O] | 613.113 [M − H]⁻ |
| 38 | 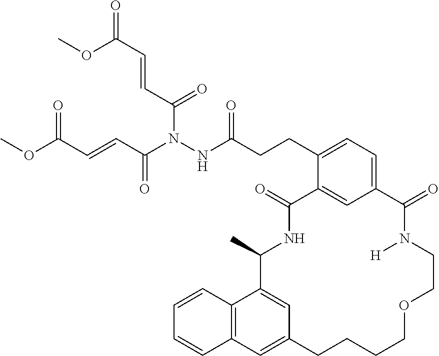 [SMILES: COC(=O)/C=C/C(=O)N(NC(=O)CCc1ccc2cc1C(=O)N[C@H](C)c1cc(cc3ccccc13)CCCCOCCNC2=O)C(=O)/C=C/C(=O)OC] | 725.108 [M − H]⁻ |

### Example 39

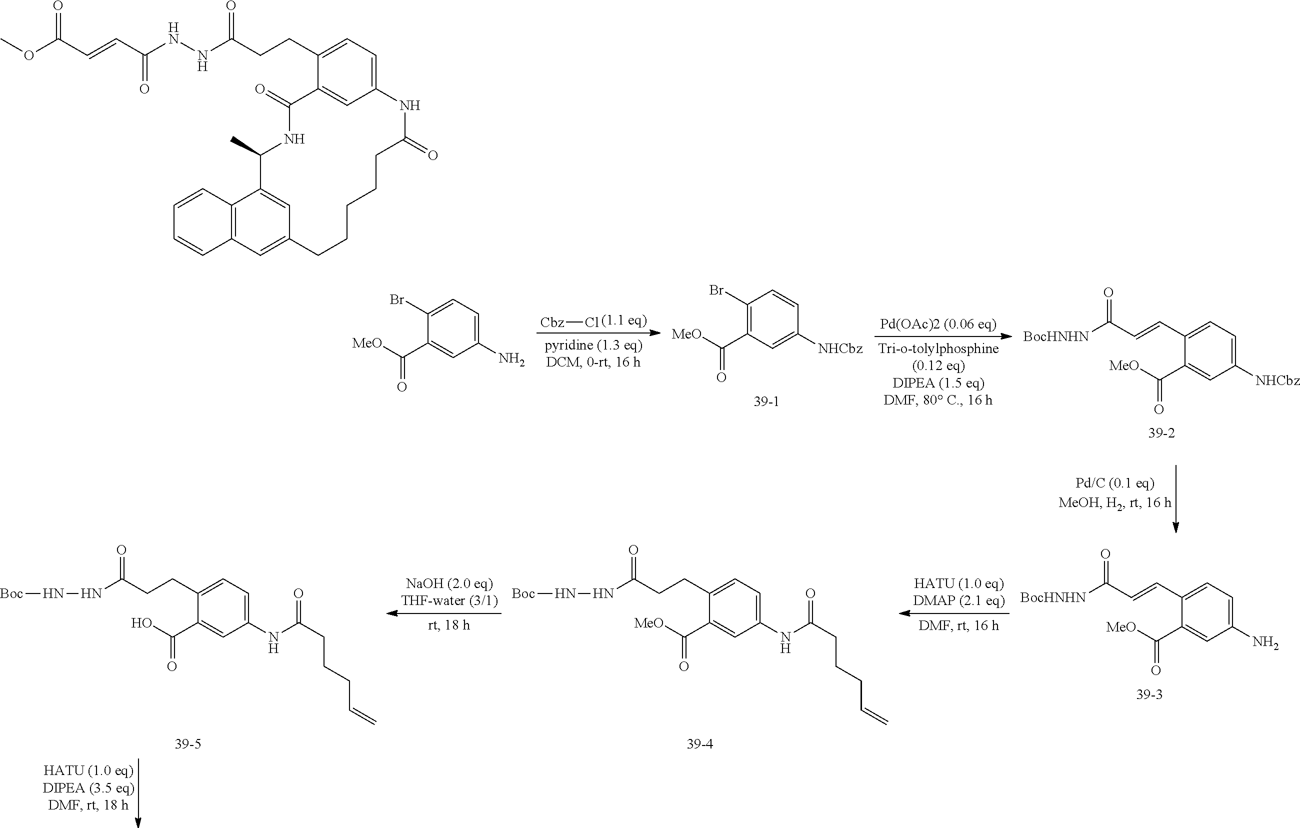

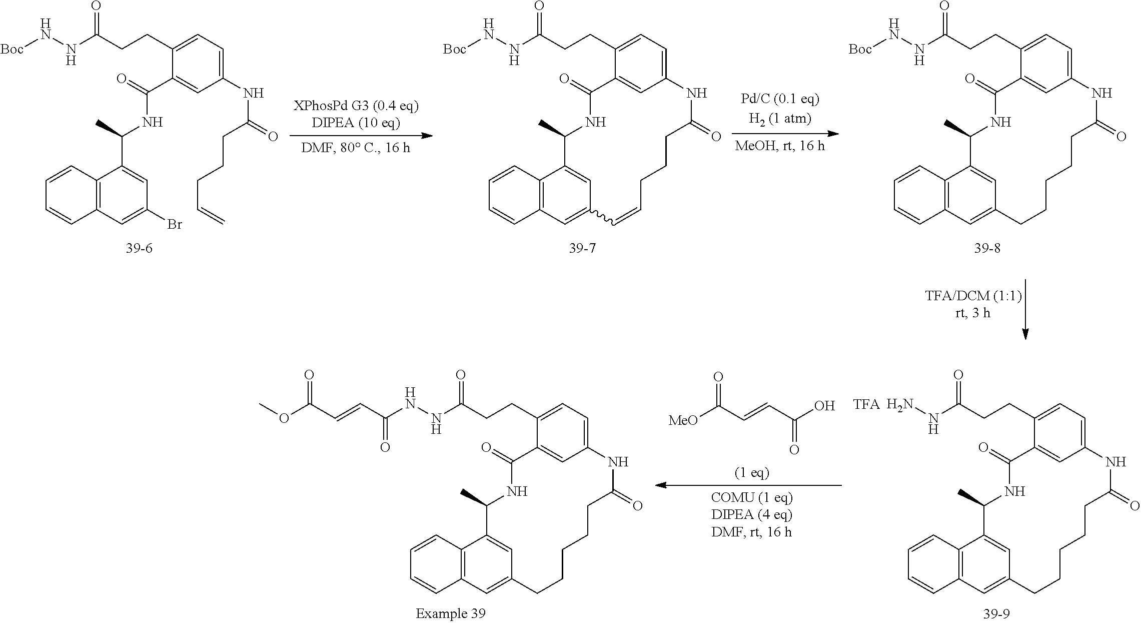 [SMILES: C=CCCCC(=O)Nc1ccc(CCC(=O)NNC(=O)OC(C)(C)C)c(C(=O)N[C@H](C)c2cc(Br)cc3ccccc23)c1 ; C[C@H]1NC(=O)c2cc(ccc2CCC(=O)NNC(=O)OC(C)(C)C)NC(=O)CCCC=Cc2cc1c1ccccc1c2 ; C[C@H]1NC(=O)c2cc(ccc2CCC(=O)NNC(=O)OC(C)(C)C)NC(=O)CCCCCc2cc1c1ccccc1c2 ; COC(=O)/C=C/C(=O)NNC(=O)CCc1ccc2cc1C(=O)N[C@H](C)c1cc(cc3ccccc13)CCCCCC(=O)N2 ; COC(=O)/C=C/C(=O)O ; C[C@H]1NC(=O)c2cc(ccc2CCC(=O)NN)NC(=O)CCCCCc2cc1c1ccccc1c2]

### Step 39-1:

A mixture of methyl 5-(benzyloxycarbonylamino)-2-bromo-benzoate (3600 mg, 9.88 mmol, 98.85% yield) and pyridine (1.20 eq, 0.97 mL, 12.0 mmol) in DCM (20 mL) at 0° C. was treated with BENZYL CHLOROFORMATE (1.00 eq, 1.4 mL, 10.0 mmol) after stirred at rt for 16 h, it was quenched with water, extracted with DCM, washed with 1N HCl. Brine, dry over Na₂SO₄, filtered, conc, column to give the desired product (3600 mg, 9.88 mmol, 99% yield). ESI MS m/z=364.02, 366.02 [M+H]⁺.

### Step 39-2:

A mixture of TRI-O-TOLYLPHOSPHINE (0.120 eq, 361 mg, 1.19 mmol), the compound from Step 39-1 (1.00 eq, 3.60 g, 9.88 mmol), tert-butyl N-(prop-2-enoylamino)carbamate (1.20 eq, 2209 mg, 11.9 mmol) and Pd(Oac)₂ (0.0600 eq, 133 mg, 0.593 mmol) and DIPEA (1.50 eq, 2.6 mL, 14.8 mmol) in DMF (20 mL) was stirred at 80° C. for 16 h, cooled to rt, diluted with EtOAc, washed with water, brine, dried over Na₂SO₄, filtered, concentrated, purified by silica gel column to give the desired product (3.94 g, 8.4 mmol, 85% yield) as a white solid.

### Step 39-3:

A suspension of the compound from Step 39-2 (1.00 eq, 2.10 g, 4.47 mmol) and Pd/C (0.200 eq, 952 mg, 0.895 mmol) in THF-MeOH (30/30 mL) was treated with H₂ (1 atm) at rt O/N. It was filtered through celite, conc, silica-gel column to give the desired product (1230 mg, 3.65 mmol, 82% yield)

### Step 39-4:

A mixture of 5-HEXENOIC ACID (1.00 eq, 0.15 mL, 1.27 mmol), the compound from Step 39-3 (1.00 eq, 430 mg, 1.27 mmol), DMAP (2.20 eq, 343 mg, 2.80 mmol) and HATU (1.00 eq, 485 mg, 1.27 mmol) in DMF (5 mL) was stirred at rt, it was diluted with EtOAc, washed with water, brine, dry over Na₂SO₄, filtered, conc, column to give the desired product (490 mg, 1.13 mmol, 89% yield)

### Step 39-5:

To a solution of the compound from Step 39-4 (1.00 eq, 490 mg, 1.13 mmol) in THF-water (3/0.5 mL) was treated with NaOH (2.00 eq, 1.1 mL, 2.26 mmol) and stirred at rt for 18 h. The mixture was acidified with 1N HCl, extracted with EtOAc. The organic layer was dried over sodium sulfate, filtered and concentrated to give the desired product (474 mg, 1.13 mmol, 100% yield).

### Step 39-6:

A mixture of the compound from Step 1-5 (1.10 eq, 338 mg, 1.18 mmol), the compound from Step 39-5 (1.00 eq, 450 mg, 1.07 mmol), DIPEA (3.80 eq, 0.71 mL, 4.08 mmol) and HATU (1.10 eq, 449 mg, 1.18 mmol) in DMF (5 mL) was stirred at rt for 16 h, diluted with EtOAc, washed with water, brine, dried over Na₂SO₄, filtered, concentrated, purified by silica gel column to give the desired product (623 mg, 0.96 mmol, 89% yield) as a white solid.

### Step 39-7:

A mixture of the compound from Step 39-6 (300 mg, 0.460 mmol), Xphos Pd G3 (156 mg, 0.184 mmol) and DIPEA (804 μl, 4.60 mmol) in DMF (24 mL) was stirred at 80° C. for 16 h. It was cooled to rt, diluted with EtOAc, filtered through celite, washed with EtOAc, the residue was washed with water, brine, dried over Na₂SO₄, filtered, conc. And purified by silica gel column to give the desired product (58 mg, 0.102 mmol, 22% yield).

### Step 39-8:

A mixture of the compound from Step 39-7 (1.00 eq, 58 mg, 0.102 mmol) and Pd/C (0.100 eq, 22 mg, 0.0102 mmol) in MeOH (4 mL) was treated with hydrogen balloon. It was filtered through celite, washed with DCM/MeOH, conc to give the desired product (50 mg, 0.0873 mmol, 86% yield)

### Step 39-9:

A mixture of the compound from Step 39-8 (1.00 eq, 50 mg, 0.0873 mmol) in DCM/TFA (1/1 mL) was stirred at rt, concentrated to give the desired product (50 mg, 0.0873 mmol, 100% yield).

### Step 39-10:

A mixture of €-4-methoxy-4-oxo-but-2-enoic acid (1.00 eq, 11 mg, 0.0873 mmol), DIPEA (4.00 eq, 0.061 mL, 0.349 mmol), the compound from Step 39-9 (1.00 eq, 41 mg, 0.0873 mmol) and COMU (1.00 eq, 37 mg, 0.0873 mmol) in DMF (1.2 mL) and stirred at rt for 16 h. The mixture was purified by HPLC to give Example 39 (15 mg, 0.0257 mmol, 29% yield) as a white solid. ESI MS m/z=585.31 [M+H]⁺.

TABLE 7
The following examples were prepared employing similar protocol as described above.

| Example | Structure | MS |
|---|---|---|
| 40 | 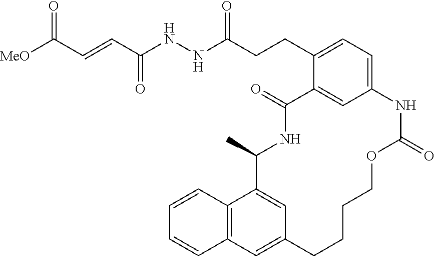 [SMILES: COC(=O)/C=C/C(=O)NNC(=O)CCc1ccc2cc1C(=O)N[C@H](C)c1cc(cc3ccccc13)CCCCOC(=O)N2] | 587.30 [M + H]⁺ |

### Example 41

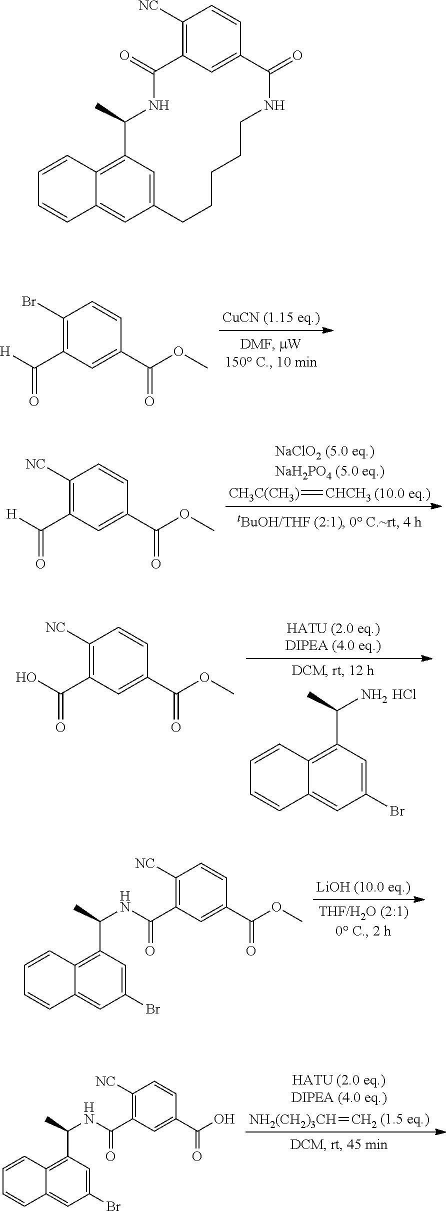

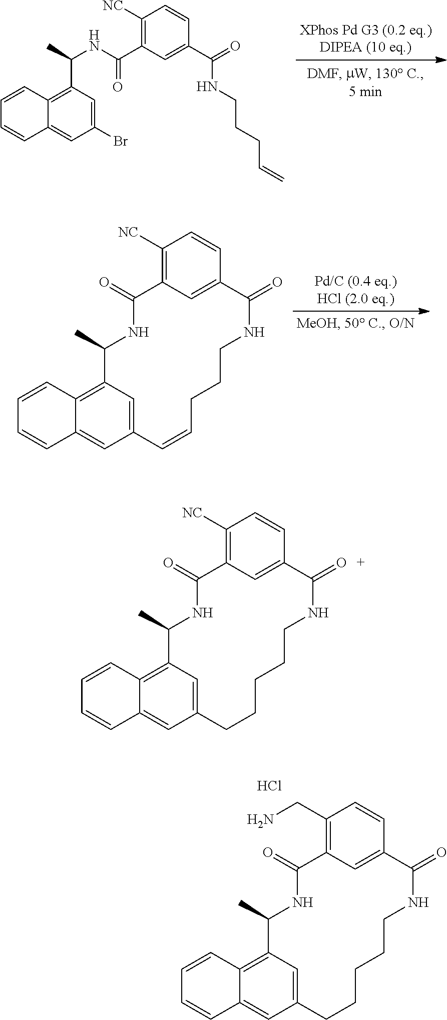

### Step 41-1:

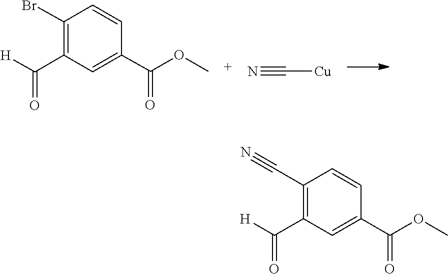

To a 20 mL microwave vial were added cyanocopper (0.422 g, 4.71 mmol), methyl 4-bromo-3-formylbenzoate (1.0 g, 4.11 mmol), and DMF (10 ml) and the sealed vial was irradiated with microwave at 150° C. for 10 min. The r×n was ˜80% conversion. Diluted with MTBE, washed with water twice and brine. Dried, filtered, concentrated and purified by CombiFlash (40 g SiO₂, EA/c-Hex: 0˜100%) to give methyl 4-cyano-3-formylbenzoate (353.8 mg, 45.5% yield). MS (m/z): 190.05 [M+H]⁺.

### Step 41-2:

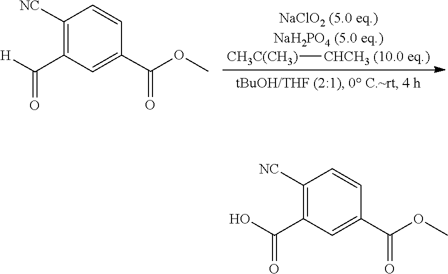

To a 250 mL round-bottomed flask containing methyl 4-cyano-3-formylbenzoate (353.5 mg, 1.869 mmol) were added tBuOH (18.69 mL), sodium dihydrogen phosphate (1121 mg, 9.34 mmol) and 2-methyl-2-butene in THF (9.343 mL, 18.69 mmol) respectively and the solution was cooled to 0° C. Freshly prepared solution of sodium chlorite (1056 mg, 9.34 mmol) (NaClO₂, 5 mL, 20% in water) was added to the mixture. The resulting solution was stirred for 4 h at 0° C. rt. Water (30 mL) was added and the aqueous layer was extracted with ethyl acetate (100 mL). Washed with brine and dried over sodium sulfate. Filtered, concentrated to give 2-cyano-5-(methoxycarbonyl)benzoic acid (383 mg, 1.867 mmol, 100% yield) as a white foam. MS (m/z): 205.99 [M+H]⁺.

### Step 41-3:

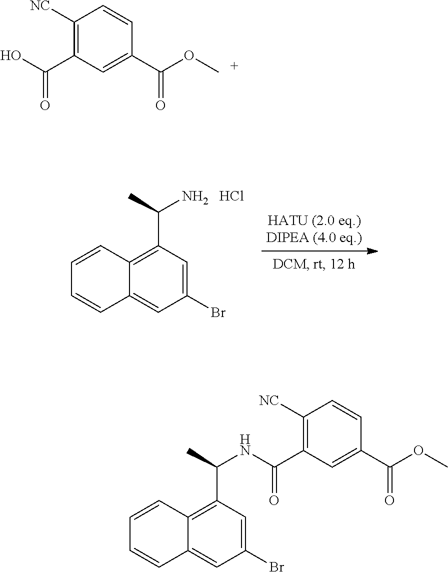 [SMILES: COC(=O)c1ccc(C#N)c(C(=O)O)c1 ; Cl ; C[C@@H](N)c1cc(Br)cc2ccccc12 ; COC(=O)c1ccc(C#N)c(C(=O)N[C@H](C)c2cc(Br)cc3ccccc23)c1]

To a 40 mL vial were added 2-cyano-5-(methoxycarbonyl)benzoic acid (383 mg, 1.867 mmol), (R)-1-(3-bromonaphthalen-1-yl)ethan-1-amine hydrochloride (696 mg, 2.427 mmol), HATU (1420 mg, 3.73 mmol), DCM (6.22 ml), and DIPEA (1304 μl, 7.47 mmol) respectively and the mixture was stirred at rt for 12 h. An amber solution was formed. LC/MS and TLC showed a complete r×n. Diluted with DCM, washed with 10% citric acid, Sat. NaHCO₃, and brine respectively. Dried, filtered, concentrated, and purified by CombiFlash (40 g SiO₂, EA/c-Hex: 0˜40%) to give methyl (R)-3-((1-(3-bromonaphthalen-1-yl)ethyl)carbamoyl)-4-cyanobenzoate (700 mg, 1.601 mmol, 86% yield). MS (m/z): 436.97 [M+H]⁺.

### Step 41-4:

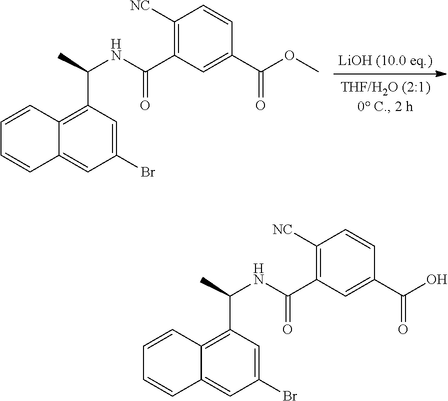 [SMILES: COC(=O)c1ccc(C#N)c(C(=O)N[C@H](C)c2cc(Br)cc3ccccc23)c1 ; C[C@@H](NC(=O)c1cc(C(=O)O)ccc1C#N)c1cc(Br)cc2ccccc12]

To a 100 mL round-bottomed flask was added a solution of methyl (R)-3-((1-(3-bromonaphthalen-1-yl)ethyl)carbamoyl)-4-cyanobenzoate (450 mg, 1.029 mmol) in THF (13.72 ml) and then the solution was cooled to 0° C. A solution of LiOH (246 mg, 10.29 mmol) in water (6.86 mL) was added dropwise and the turbid mixture was stirred at 0° C. for 2 h. Acidified with HCl (3773 μL, 11.32 mmol), extracted with EtOAc, dried, filtered, concentrated to give (R)-3-((1-(3-bromonaphthalen-1-yl)ethyl)carbamoyl)-4-cyanobenzoic acid (450 mg, 1.063 mmol, 103% yield) as a yellow foam. MS (m/z): 422.97 [M+H]⁺.

### Step 41-5:

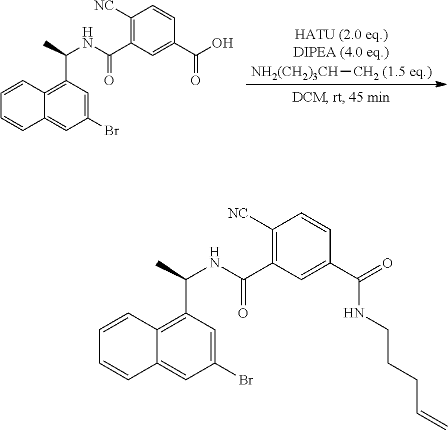

To a 40 mL vial containing (R)-3-((1-(3-bromonaphthalen-1-yl)ethyl)carbamoyl)-4-cyanobenzoic acid (450 mg, 1.063 mmol) were added HATU (1420 mg, 3.73 mmol), pent-4-en-1-amine (136 mg, 1.595 mmol), DCM (6.22 ml), and DIPEA (1304 μl, 7.47 mmol) respectively and the mixture was stirred at rt for 45 min. A yellow solution was formed. LC/MS and TLC showed a complete r×n. Diluted with DCM, washed with 10% citric acid, Sat. NaHCO3, and brine respectively. Dried, filtered, concentrated, and purified by CombiFlash (40 g SiO₂, EA/c-Hex: 0˜40%) to give (R)-N3-(1-(3-bromonaphthalen-1-yl)ethyl)-4-cyano-N1-(pent-4-en-1-yl)isophthalamide (367 mg, 70.4% yield). MS (m/z): 490.11[M+H]⁺.

### Step 41-6:

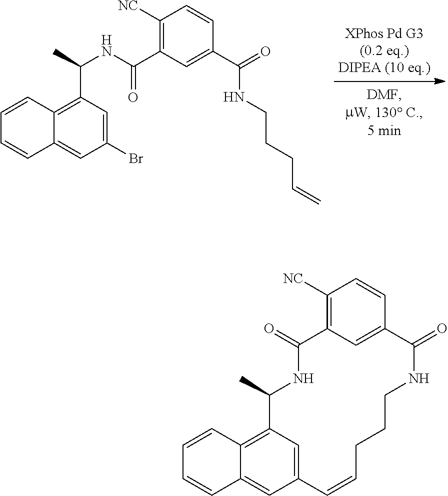 [SMILES: C=CCCCNC(=O)c1ccc(C#N)c(C(=O)N[C@H](C)c2cc(Br)cc3ccccc23)c1 ; C[C@H]1NC(=O)c2cc(ccc2C#N)C(=O)NCCC/C=C\c2cc1c1ccccc1c2]

To a 20 mL microwave vial were added JL-05004-12 (80 mg, 0.163 mmol), XPhos Pd G3 (41 mg, 0.048 mmol), DMF (10 ml), and Hunig's base (285 μl, 1.631 mmol) and the colorless solution was irradiated under microwave at 130° C. for 5 min. LC/MS indicated r×n complete. Additional 3 runs were carried out parallelly. Combined all the runs, diluted with EtOAc, washed with water and brine. Dried, filtered, concentrated, and purified by CombiFlash (24 g SiO₂, MeOH/DCM: 0˜20% contains 1% TEA) to give (R,Z)-2-methyl-4,6-dioxo-3,7-diaza-1(1,3)-naphthalena-5(1,3)-benzenacyclododecaphan-11-ene-56-carbonitrile (149 mg) as an orange foam. MS (m/z): 408.17 [M−H]⁻.

### Step 41-7:

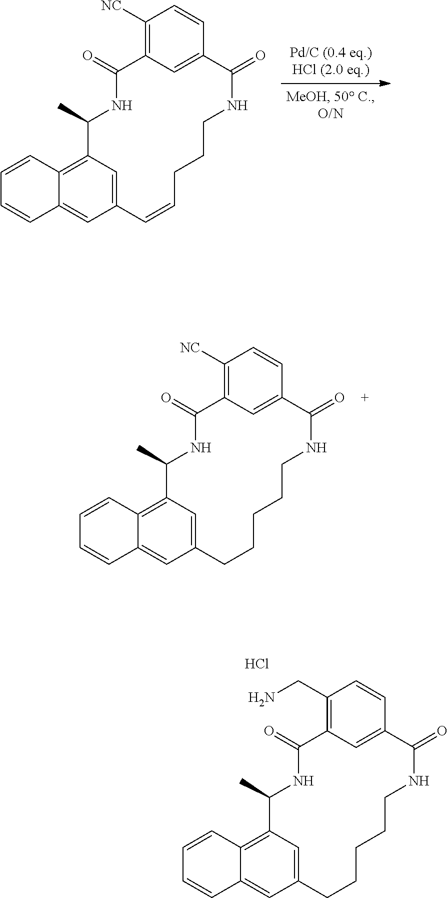 [SMILES: C[C@H]1NC(=O)c2cc(ccc2C#N)C(=O)NCCC/C=C\c2cc1c1ccccc1c2 ; C[C@H]1NC(=O)c2cc(ccc2C#N)C(=O)NCCCCCc2cc1c1ccccc1c2 ; Cl ; C[C@H]1NC(=O)c2cc(ccc2CN)C(=O)NCCCCCc2cc1c1ccccc1c2]

To a 2-dram vial were added (R,Z)-2-methyl-4,6-dioxo-3,7-diaza-1(1,3)-naphthalena-5(1,3)-benzenacyclododecaphan-11-ene-56-carbonitrile (70 mg, 0.171 mmol), Pd/C (72.8 mg, 0.068 mmol), and MeOH (4.5 mL) and the suspension was stirred at rt under 1 atm H₂ atmosphere for 30 min. Some starting material was not fully dissolved. HCl (85 μl, 0.342 mmol) was added and quickly the undissolved starting material disappeared. The suspension was stirred at rt O/N. Filtered through a pad of celite and concentrated in dryness to give an inseparable mixture (58 mg) of (R)-2-methyl-4,6-dioxo-3,7-diaza-1(1,3)-naphthalena-5(1,3)-benzenacyclododecaphane-5⁶-carbonitrile and (R)-5⁶-(aminomethyl)-2-methyl-3,7-diaza-1(1,3)-naphthalena-5(1,3)-benzenacyclododecaphane-4,6-dione. MS (m/z): 410.19 [M−H]⁻ and 414.21 [M−H]⁻ respectively.

### Example 42

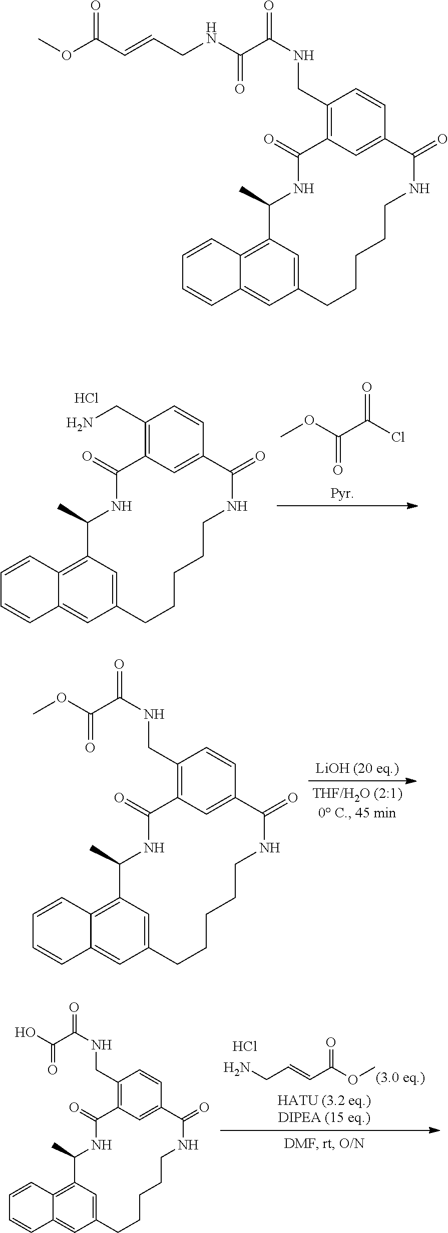

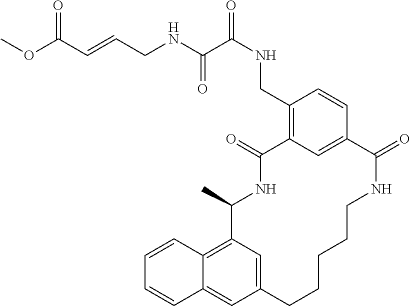 [SMILES: COC(=O)/C=C/CNC(=O)C(=O)NCc1ccc2cc1C(=O)N[C@H](C)c1cc(cc3ccccc13)CCCCCNC2=O]

### Step 42-1:

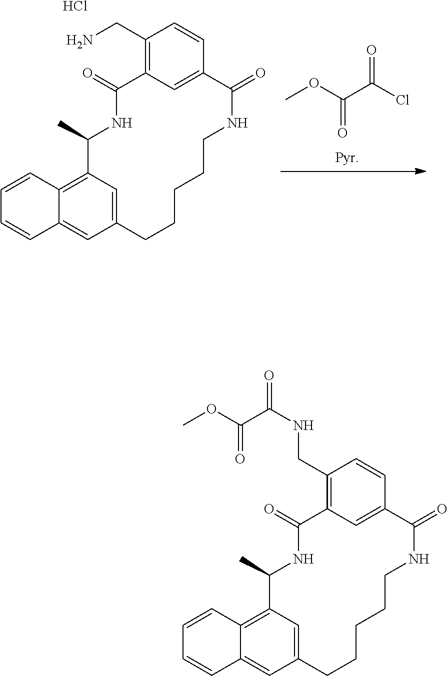

To a 2-dram vial containing (R)-5⁶-(aminomethyl)-2-methyl-3,7-diaza-1(1,3)-naphthalena-5(1,3)-benzenacyclododecaphane-4,6-dione hydrochloride (73 mg, 0.176 mmol) was added DCM (2 mL) and the solution was cooled to 0° C. Methyl 2-chloro-2-oxoacetate (19.39 μL, 0.211 mmol) and pyridine (71.0 μL, 0.878 mmol) were added and the solution was stirred at 0° C. for 1 h and rt for 2 h. Diluted with DCM and washed with brine. Dried, filtered, concentrated and purified by CombiFlash (4 g SiO₂, ACE/c-Hex: 0˜50%) to give methyl (R)-2-(((2-methyl-4,6-dioxo-3,7-diaza-1(1,3)-naphthalena-5(1,3)-benzenacyclododecaphane-5⁶-yl)methyl)amino)-2-oxoacetate (5.3 mg, 6.01% yield) as a white solid. MS (m/z): 502.23 [M+H]⁺

### Step 42-2:

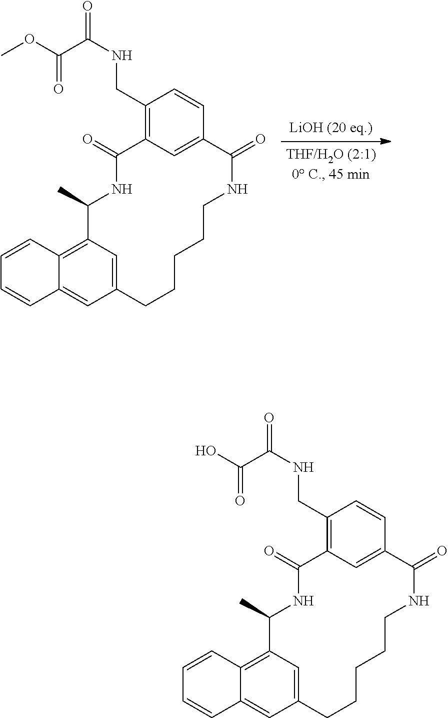 [SMILES: COC(=O)C(=O)NCc1ccc2cc1C(=O)N[C@H](C)c1cc(cc3ccccc13)CCCCCNC2=O ; C[C@H]1NC(=O)c2cc(ccc2CNC(=O)C(=O)O)C(=O)NCCCCCc2cc1c1ccccc1c2]

To a 2-dram vial containing methyl (R)-2-(((2-methyl-4,6-dioxo-3,7-diaza-1(1,3)-naphthalena-5(1,3)-benzenacyclododecaphane-5⁶-yl)methyl)amino)-2-oxoacetate (5.3 mg, 10.57 μmol) was added THF (0.704 mL) and the solution was cooled to 0° C. followed by addition of a solution of LiOH (5.5 mg, 0.230 mmol) in water (0.352 mL) dropwise. The r×n was stirred at 0° C. for 45 min. RXN was complete by TLC and LC/MS. HCl (81 μl, 0.243 mmol) was added dropwise and the solution was diluted with ACN and concentrated to give (R)-2-(((2-methyl-4,6-dioxo-3,7-diaza-1(1,3)-naphthalena-5(1,3)-benzenacyclododecaphane-5⁶-yl)methyl)amino)-2-oxoacetic acid (5.2 mg). Used directly for the next step. MS (m/z): 486.20 [M−H]⁻

### Step 42-3:

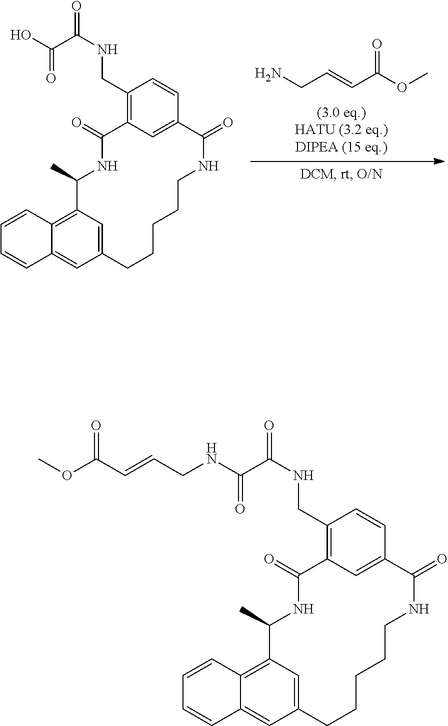

To a 2-dram vial containing (R)-2-(((2-methyl-4,6-dioxo-3,7-diaza-1(1,3)-naphthalena-5(1,3)-benzenacyclododecaphane-5⁶-yl)methyl)amino)-2-oxoacetic acid (5.2 mg, 10.67 μmol) were added methyl (E)-4-aminobut-2-enoate hydrochloride (4.85 mg, 0.032 mmol), HATU (13 mg, 0.034 mmol), DCM (2 mL), and hunig's base (30 μL, 0.172 mmol) respectively and the suspension was stirred at rt for 2 h. Directly loaded on a small cartridge and purified by prep TLC (7.5% MeOH/DCM) to give methyl (R,E)-4-(2-(((2-methyl-4,6-dioxo-3,7-diaza-1(1,3)-naphthalena-5(1,3)-benzenacyclododecaphane-5⁶-yl)methyl)amino)-2-oxoacetamido)but-2-enoate. MS (m/z): 583.25 [M−H]⁻

### Biological Activity

SARS-CoV-2 Papain-like (PLpro) protease biochemical enzyme inhibition assay: Ubiquitin-modified at the C-terminal with a masked fluorophore is used as a substrate for PLpro which cleaves the fluorophore and the unmasked free fluorophore generates a fluorescent signal. The fluorophore is either Rhodamine or AMC. A similar assay is also explored using ISG15-AMC. Procedure: 1) Transfer compounds to 384w assay plates using Echo according to the general platemaps. 2) Add 10 μL of 1×Assay Buffer to LOW control wells and 10 μL of 2×PLpro solution to other wells in the assay plate according to the platemap spin at 800 rpm for 1 min and incubate at RT for 30 mins. 3) Add 10 μL of 2×substrate solution to each well of enzyme solution according to platemap. Spin at 800 rpm for 1 min and incubate assay at RT in the dark for 30 mins. 4) Spin at 800 rpm and read plates on Virology Envision for Ub-R110 the Ex/Em was 485/535 nm. 5) Normalize data to high and low controls and determine IC50 by fitting data to normalized response versus inhibitor (variable slope) using GraphPad Prism 7. All experiments were run in duplicate, and IC₅₀ ranges are reported as follows: A<0.1 □M; B 0.1-1 □M; C 1-10 □M; D>10 □M.

SARS-CoV-2 Replicon Assay (Huh-7): A SARS-CoV-2 replicon expressing a Renilla luciferase reporter and rendered non-infectious due to deletions in the S, E, M, 3a, 3b, 6, 7a, 7b, and 8 open reading frames was utilized for evaluating compound activity against the autologous SARS-CoV-2 proteins in a cell-based assay. The SARS-CoV-2 replicon construct is a single bacterial artificial chromosome (BAC) encoded fragment (Codex DNA). The replicon fragment is amplified and linearized from the BAC by PCR using Platinum SuperFi II PCR Master Mix (Invitrogen) and the following primers: forward 5′-CGC ACG GTT ATG TGG ACC CTG-3′ and reverse 5′-TTT TTT TTT TTT TTT TTT TTT TTT TTT TGT CAT TCT CCT AAG AAG CTA TTA-3′. SARS-CoV-2 replicon RNA is synthesized by in vitro transcription using mMESSAGE mMACHINE T7 Ultra (Invitrogen). A plasmid encoding codon-optimized SARS-CoV-2N is linearized by restriction digestion and used as a template for in vitro transcription using mMESSAGE mMACHINE T7 Ultra to produce SARS-CoV-2N RNA. RNA is purified using the Monarch RNA cleanup kit (New England Biolabs). White 384-well tissue-culture-treated clear-bottom plates are used in this assay. Using a Labcyte ECHO liquid dispenser, 3-fold serial dilutions of compounds suspended in DMSO are added to the inner 308 wells in duplicate in a total volume of 125 nL per well. Two columns are treated only with 125 nL DMSO, to be used as controls. In a typical assay, 650 ng SARS-CoV-2 replicon RNA and 650 ng codon-optimized SARS-CoV-2N RNA are mixed with 100 μL of HuH-7 cells suspended in Buffer R (Neon Transfection System, Invitrogen) at a final concentration of 1E7 cells/mL and co-electroporated using the Neon Transfection System at 1700V-20 ms-1 pulse. HuH-7 cells at 1E7 cells/mL without RNA are electroporated as a low control. For each 100 μL electroporated cell suspension, 1150 μL pre-warmed (37° C.) media (DMEM, 1×GlutaMAX, 10% FBS) is added to the cells for a final concentration of 800 cells/μL. The electroporated cells are seeded into the 308 inner compound and control wells of the 384-well plate at 20,000 cells per well in a total volume of 25 μL. One drug-free DMSO-treated column of wells is seeded with cells electroporated with replicon and N RNA as a high control. Another drug-free DMSO-treated column of wells is seeded with cells electroporated without RNA as a low control. The unused outer wells are filled with 25 μL of moat media containing 1% penicillin/streptomycin. Plates are incubated at 37° C. in a CO₂ humidity-controlled incubator for approximately 20 hours, then brought to room temperature. 25 μL of room temperature 1×Renilla-Glo Luciferase Assay Reagent (Promega) are added to each well and incubated at room temperature for 10 minutes and luminescence was measured on a Perkin Elmer EnVision. The Renilla-Glo reagent quantifies the amount of luciferase activity, which gives a measure of replicon activity present. The half-maximal effective concentration (EC₅₀) was determined using GraphPad Prism. Percent residual activity of the replicon is determined after normalizing the curve to the mean RLU high control at 100% and the mean RLU low control at 0%. EC₅₀ curves are generated using a variable slope four-parameter logistic model with equation Y=100/(1+X^{∧}HillSlope)/(EC₅₀^{∧}HillSlope). A<1 □M; B 1-10 □M; C 10-50 □M; D>50 □M.

SARS-CoV-2 Cellular Assay (Vero 76): Test compounds are serially diluted using eight half-log dilutions in test medium (MEM supplemented with 2% FBS and 50 pg/mL gentamicin). Each dilution is added to 5 wells of a 96-well plate with 80-100% confluent Vero 76 cells. Three wells of each dilution are infected with virus (SARS-CoV-2 USA-WA1/2020), and two wells remain uninfected as toxicity controls. Six wells are infected and untreated as virus controls, and six wells are uninfected and untreated as cell controls. Viruses are prepared to achieve the lowest possible multiplicity of infection (MOI˜0.002) that would yield >80% cytopathic effect (CPE) at 6 days. Plates are incubated at 37±2° C., 5% CO₂. For neutral red assay, on day 6 post-infection, once untreated virus control wells reach maximum CPE, plates are stained with neutral red dye for approximately 2 hours (±15 minutes). Supernatant dye is removed, and wells are rinsed with PBS, and the incorporated dye is extracted in 50:50 Sorensen citrate buffer/ethanol for >30 minutes and the optical density is read on a spectrophotometer at 540 nm. Optical densities are converted to percent of cell controls and normalized to the virus control, then the concentration of test compound required to inhibit CPE by 50% (EC₅₀) is calculated by regression analysis. The concentration of compound that would cause 50% cell death in the absence of virus was similarly calculated (CC₅₀). EC₅₀ ranges are reported as follows: A<1 □M; B 1-10 □M; C 10-50 □M; D>50 □M.

TABLE 8
Summary of Activities

| | | | SARS-COV-2 |
|---|---|---|---|
| Example # | PLpro IC₅₀ | SARS-COV-2 replicon EC₅₀ | BSL3 EC₅₀ |
| 1 | D | — | — |
| 2 | B | B | B |
| 3 | D | — | — |
| 4 | C | — | — |
| 5 | B | B | — |
| 6 | D | — | — |
| 7 | C | — | — |
| 8 | D | — | — |
| 9 | D | — | — |
| 11 | D | — | — |
| 12 | D | — | — |
| 13 | D | — | — |
| 14 | B | D | — |
| 15 | C | — | — |
| 16 | D | — | — |
| 17 | B | C | — |
| 18 | B | C | B |
| 19 | A | B | — |
| 20 | D | — | — |
| 21 | D | — | — |
| 22 | C | — | — |
| 23 | A | C | — |
| 24 | A | C | |
| 25 | — | A | A |
| 26 | A | — | — |
| 27 | A | — | — |
| 28 | A | — | — |
| 29 | A | — | — |
| 30 | A | B | A |
| 31 | A | — | — |
| 32 | A | — | — |
| 33 | A | B | A |
| 34 | B | — | — |
| 35 | A | — | — |
| 36 | B | — | — |
| 37 | A | A | A |
| 38 | A | — | — |
| 39 | A | B | A |
| 40 | A | B | A |
| 41 | C | — | — |
| 42 | D | — | — |

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

1. A compound represented by Formula (I), or a pharmaceutically acceptable salt thereof, 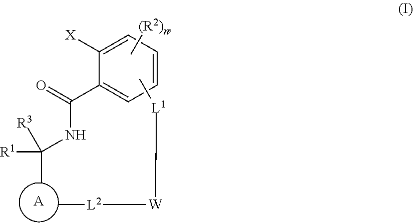
wherein:
A is selected from the group consisting of optionally substituted aryl, optionally substituted heteroaryl, optionally substituted —C₃-C₈ cycloalkyl, and optionally substituted 3- to 8-membered heterocycloalkyl;
R¹ and R³ are each independently selected from the group consisting of hydrogen, optionally substituted —C₁-C₄ alkyl, optionally substituted —C₂-C₄ alkenyl, optionally substituted —C₂-C₄ alkynyl, and optionally substituted —C₃-C₆ cycloalkyl;
alternatively, R¹ and R³ are taken together with the carbon atom to which they are attached to form an optionally substituted 3- to 8-membered carbocyclic or a 3- to 8-membered heterocyclic ring;
each R² is selected from the group consisting of halogen, —OR¹¹, —OC(O)R¹¹, —C(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
R¹¹ is selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
R¹² and R¹³ at each occurrence are independently selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; alternatively R¹² and R¹³ are taken together with the nitrogen atom to which they are attached to form an optionally substituted 3- to 8-membered heterocyclic ring;
n is 0, 1, 2, or 3;
L¹ and L² are independently selected from the group consisting of —CR²¹═CR²²—, —CR²¹R²³—CR²²R²⁴—, —(CR²¹R²³)ₘ—O—, —(CR²¹R²³)ₘ—S—, —(CR²¹R²³)ₘ—NR¹²—, 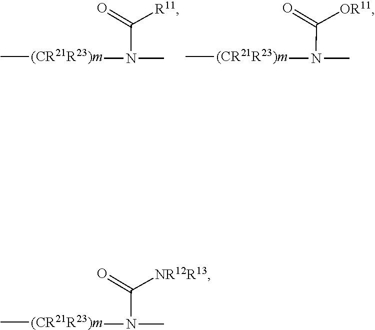
—(CR²¹R²³)ₘ—NR¹²C(O)—, —(CR²¹R²³)ₘ—NR¹²C(O)O—, —(CR²¹R²³)ₘ—NR¹²C(O)NR¹³—, —(CR²¹R²³)ₘ—C(O)N(R¹²)—, —(CR²¹R²³)ₘ—C(O)—, —(CR²¹R²³)ₘ—S(O)₂—, —(CR²¹R²³)ₘ—S(O)(NH)—, —(CR²¹R²³)ₘ—S(O)₂NR¹²—, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
m is 0, 1, 2, 3 or 4;
R²¹ and R²² at each occurrence are independently selected from the group consisting of hydrogen, halogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
R²³ and R²⁴ at each occurrence are independently selected from the group consisting of hydrogen, halogen, —OR¹¹, —OC(O)R¹¹, —OC(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
W is selected from the group consisting of optionally substituted —C₁-C₈ alkyl, optionally substituted —C₃-C₈ cycloalkyl, optionally substituted 3- to 8-membered heterocycloalkyl, optionally substituted aryl, optionally substituted arylalkyl, optionally substituted heteroaryl, optionally substituted heteroarylalkyl; —(CR²¹R²³)_{q1}—O—(CR²²R²⁴)_{q2}—, —(CR²¹R²³)_{q1}—NR¹²—(CR²²R²⁴)_{q2}—, and —(CR²¹R²³)_{q1}—S—(CR²²R²⁴)_{q2}—;
q1 is 0, 1, 2, 3, 4, 5, 6, 7, or 8;
q2 is 0, 1, 2, 3, 4, 5, 6, 7, or 8;
X is selected from the group consisting of halogen, optionally substituted —C₁-C₆ alkyl, optionally substituted —C₂-C₆ alkenyl, optionally substituted —C₃-C₆ cycloalkyl, 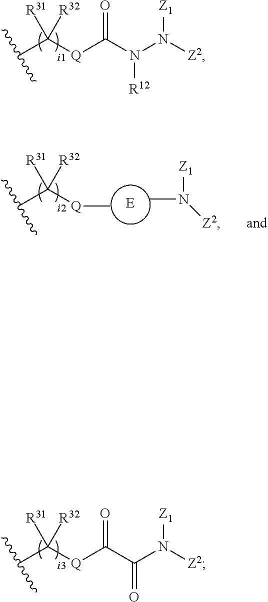
Z¹ and Z² are each independently an amino protecting group, R¹³, 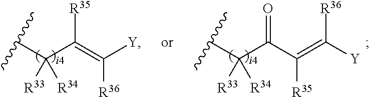
R³¹, R³², R³³ and R³⁴ at each occurrence are independently selected from the group consisting of hydrogen, optionally substituted —CH₃, and halogen;
R³⁵ and R³⁶ at each occurrence are each independently selected from the group consisting of hydrogen, optionally substituted —CH₃, halogen, and —CN;
i1, i2, i3, and i4 are each independently 0, 1, 2, 3, 4;
Q is selected from absent, —O—, and —NR¹²—;
E is selected from optionally substituted aryl, optionally substituted heteroaryl; and
Y is selected from the group consisting of hydrogen, halogen, —CF₃; —CN, —C(O)R¹¹, —C(O)OR¹², —C(O)NR¹²R¹³, —S(O)₂R¹¹, —S(O)₂NR¹²R¹³, optionally substituted aryl, and optionally substituted heteroaryl.

2. The compound of claim 1, wherein R¹ is methyl or —CD₃, R3 is hydrogen, and X is methyl, —CD₃,

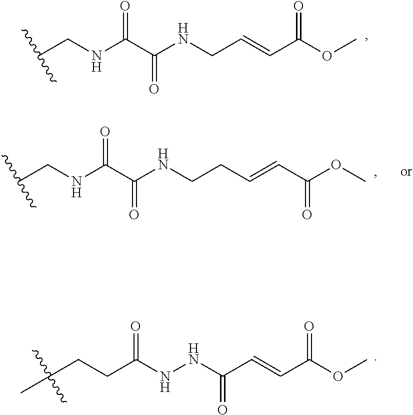 [SMILES: *CNC(=O)C(=O)NC/C=C/C(=O)OC ; *CNC(=O)C(=O)NCC/C=C/C(=O)OC ; *CCC(=O)NNC(=O)/C=C/C(=O)OC]

3. The compound of claim 1, represented by one of Formulae (IV-1)˜(IV-8), or a pharmaceutically acceptable salt thereof: 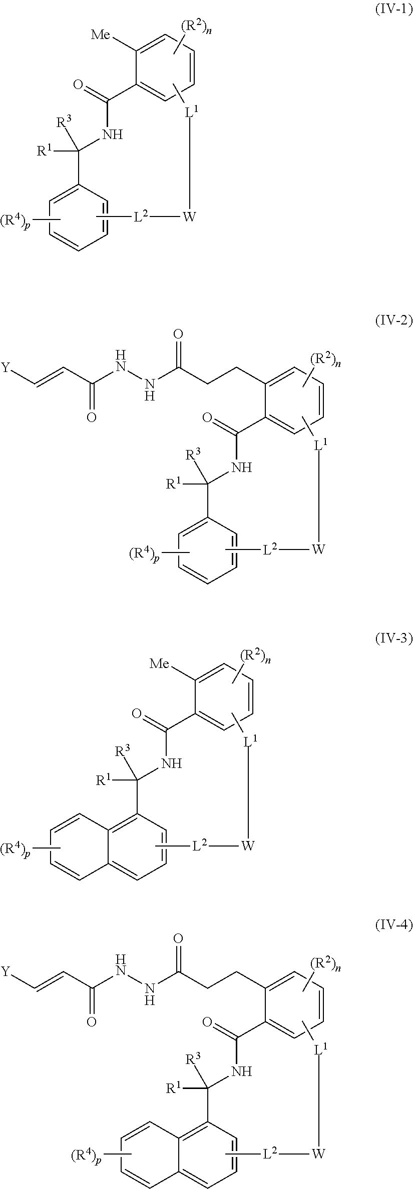 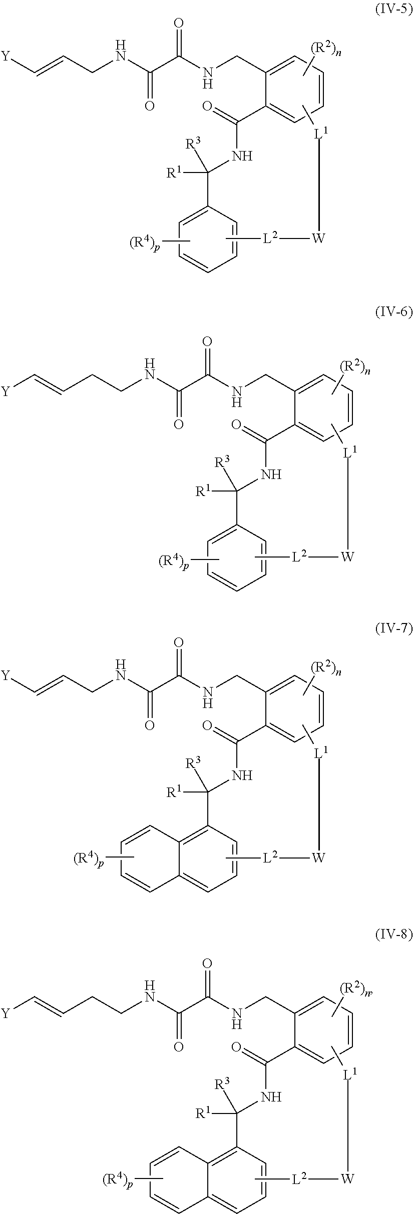
wherein each R⁴ is selected from the group consisting of halogen, —OR¹¹, —OC(O)R¹¹, —C(O)OR¹¹, —OC(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; p is 0, 1, 2, 3, or 4; and Y, R¹, R², R³, R¹¹, R¹², R¹³, L¹, L², W, and n are as defined in claim 1.

4. The compound of claim 1, represented by one of Formulae (V-1)˜(V-8), or a pharmaceutically acceptable salt thereof: 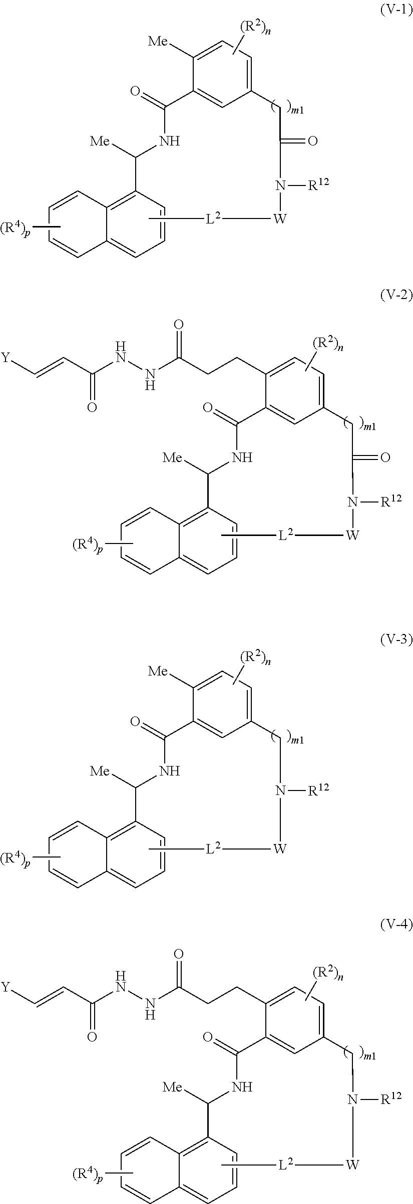 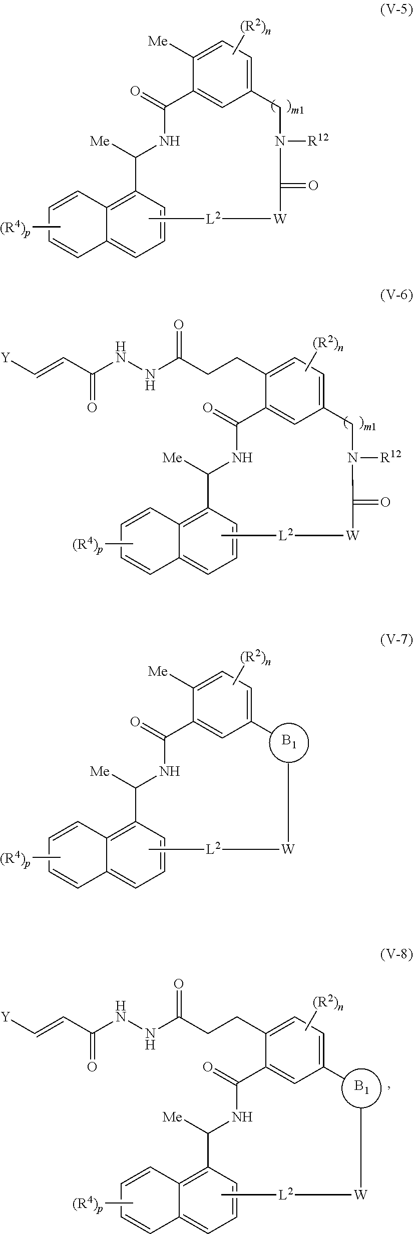
wherein each R⁴ is selected from the group consisting of halogen, —OR¹¹, —OC(O)R¹¹, —C(O)OR¹¹, —OC(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; p is 0, 1, 2, 3, or 4; B₁ is selected from the group consisting of optionally substituted aryl, optionally substituted heteroaryl, optionally substituted —C₃-C₈ cycloalkyl, and optionally substituted 3- to 8-membered heterocycloalkyl; m1 is 0, 1 or 2; and Y, R², R¹¹, R¹², R¹³, L², W, and n are as defined in claim 1.

5. The compound of claim 1, represented by one of Formulae (XII-1)˜(XII-8), or a pharmaceutically acceptable salt thereof: 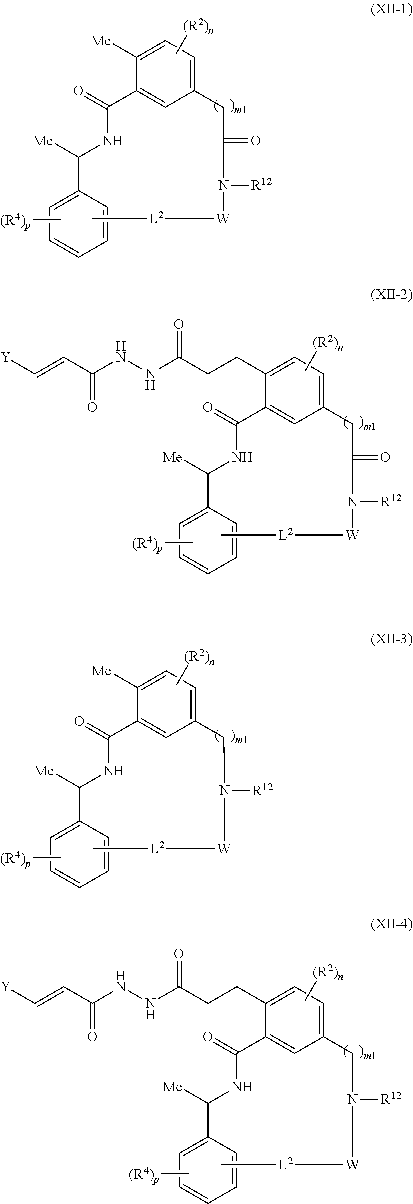 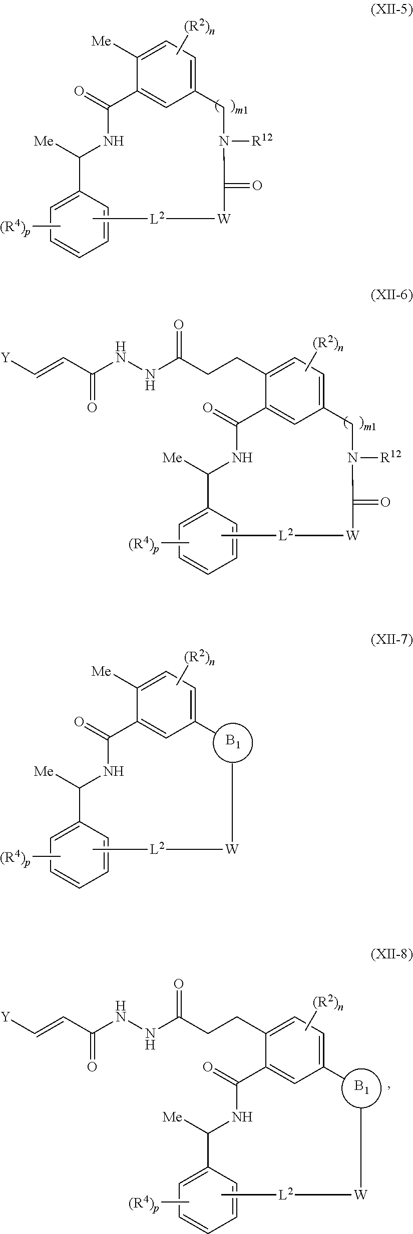
wherein each R⁴ is selected from the group consisting of halogen, —OR¹¹, —OC(O)R¹¹, —C(O)OR¹¹, —OC(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; p is 0, 1, 2, 3, or 4; B₁ is selected from the group consisting of optionally substituted aryl, optionally substituted heteroaryl, optionally substituted —C₃-C₈ cycloalkyl, and optionally substituted 3- to 8-membered heterocycloalkyl; m1 is 0, 1 or 2; and Y, R², R¹¹, R¹², R¹³, L², W, and n are as defined in claim 1.

6. The compound of claim 1, selected from the compounds set forth below or a pharmaceutically acceptable salt thereof:

| Compound | Structure | Compound | Structure |
|---|---|---|---|
| 1 | 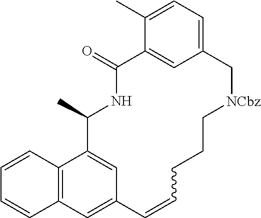 [SMILES: Cc1ccc2cc1C(=O)N[C@H](C)c1cc(cc3ccccc13)C=CCCCN(C(=O)OCc1ccccc1)C2] | 2 | 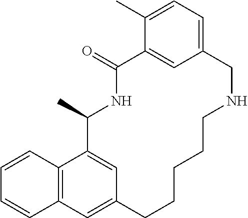 [SMILES: Cc1ccc2cc1C(=O)N[C@H](C)c1cc(cc3ccccc13)CCCCCNC2] |
| 3 | 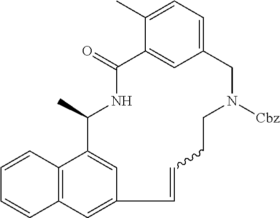 [SMILES: Cc1ccc2cc1C(=O)N[C@H](C)c1cc(cc3ccccc13)C=CCCN(C(=O)OCc1ccccc1)C2] | 4 | 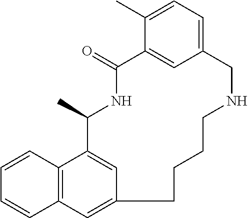 [SMILES: Cc1ccc2cc1C(=O)N[C@H](C)c1cc(cc3ccccc13)CCCCNC2] |
| 5 | 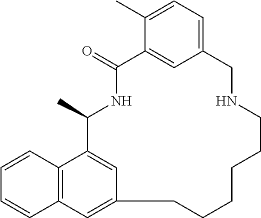 [SMILES: Cc1ccc2cc1C(=O)N[C@H](C)c1cc(cc3ccccc13)CCCCCCNC2] | 6 | 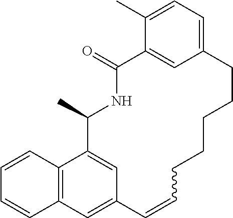 [SMILES: Cc1ccc2cc1C(=O)N[C@H](C)c1cc(cc3ccccc13)C=CCCCCC2] |
| 7 | 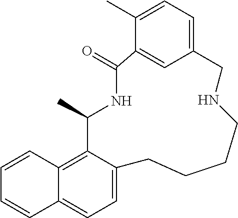 [SMILES: Cc1ccc2cc1C(=O)N[C@H](C)c1c(ccc3ccccc13)CCCCNC2] | 8 | 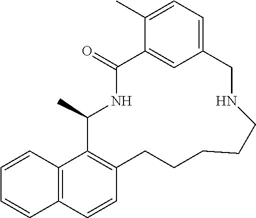 [SMILES: Cc1ccc2cc1C(=O)N[C@H](C)c1c(ccc3ccccc13)CCCCCNC2] |
| 9 | 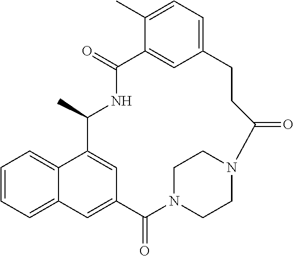 [SMILES: Cc1ccc2cc1C(=O)N[C@H](C)c1cc(cc3ccccc13)C(=O)N1CCN(CC1)C(=O)CC2] | | |
| 11 | 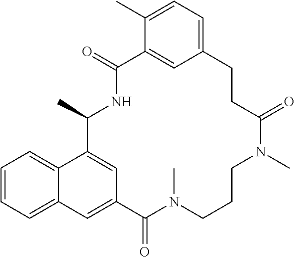 [SMILES: Cc1ccc2cc1C(=O)N[C@H](C)c1cc(cc3ccccc13)C(=O)N(C)CCCN(C)C(=O)CC2] | 12 | 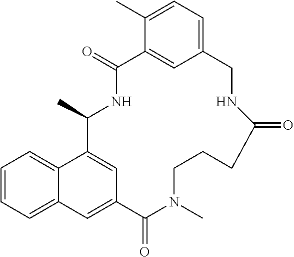 [SMILES: Cc1ccc2cc1C(=O)N[C@H](C)c1cc(cc3ccccc13)C(=O)N(C)CCCC(=O)NC2] |
| 13 | 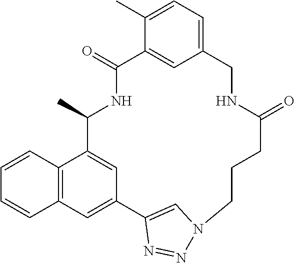 [SMILES: Cc1ccc2cc1C(=O)N[C@H](C)c1cc(cc3ccccc13)-c1cn(nn1)CCCC(=O)NC2] | 14 | 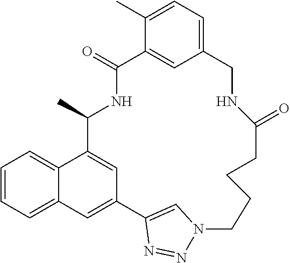 [SMILES: Cc1ccc2cc1C(=O)N[C@H](C)c1cc(cc3ccccc13)-c1cn(nn1)CCCCC(=O)NC2] |
| 15 | 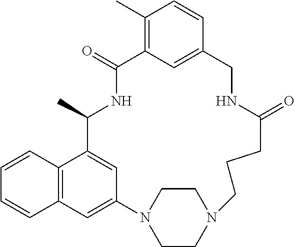 [SMILES: Cc1ccc2cc1C(=O)N[C@H](C)c1cc(cc3ccccc13)N1CCN(CCCC(=O)NC2)CC1] | 16 | 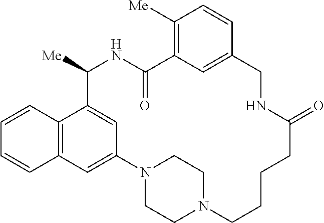 [SMILES: Cc1ccc2cc1C(=O)N[C@H](C)c1cc(cc3ccccc13)N1CCN(CCCCC(=O)NC2)CC1] |
| 17 | 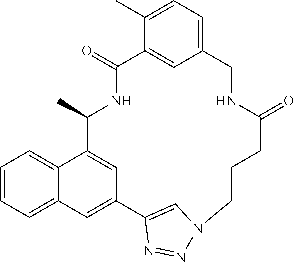 [SMILES: Cc1ccc2cc1C(=O)N[C@H](C)c1cc(cc3ccccc13)-c1cn(nn1)CCCC(=O)NC2] | 18 | 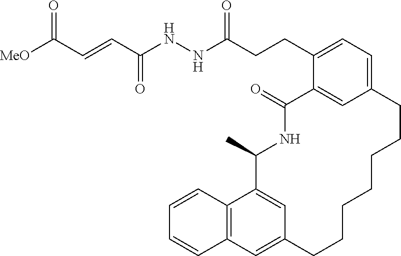 [SMILES: COC(=O)/C=C/C(=O)NNC(=O)CCc1ccc2cc1C(=O)N[C@H](C)c1cc(cc3ccccc13)CCCCCCC2] |
| 19 | 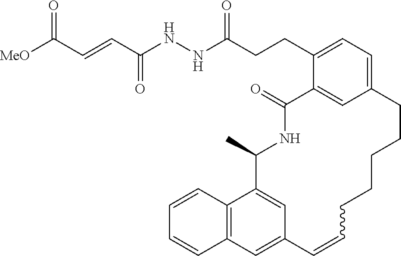 [SMILES: COC(=O)/C=C/C(=O)NNC(=O)CCc1ccc2cc1C(=O)N[C@H](C)c1cc(cc3ccccc13)C=CCCCCC2] | 20 | 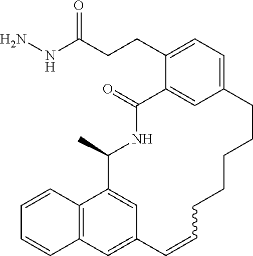 [SMILES: C[C@H]1NC(=O)c2cc(ccc2CCC(=O)NN)CCCCCC=Cc2cc1c1ccccc1c2] |
| 21 | 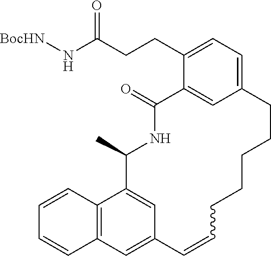 [SMILES: C[C@H]1NC(=O)c2cc(ccc2CCC(=O)NNC(=O)OC(C)(C)C)CCCCCC=Cc2cc1c1ccccc1c2] | 22 | 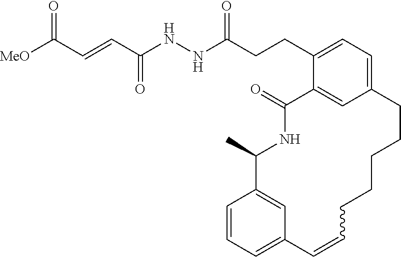 [SMILES: COC(=O)/C=C/C(=O)NNC(=O)CCc1ccc2cc1C(=O)N[C@H](C)c1cccc(c1)C=CCCCCC2] |
| 23 | 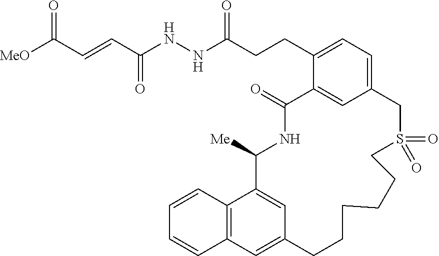 [SMILES: COC(=O)/C=C/C(=O)NNC(=O)CCc1ccc2cc1C(=O)N[C@H](C)c1cc(cc3ccccc13)CCCCCCS(=O)(=O)C2] | 24 | 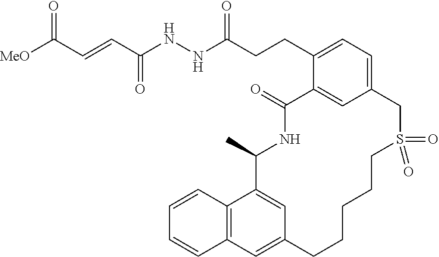 [SMILES: COC(=O)/C=C/C(=O)NNC(=O)CCc1ccc2cc1C(=O)N[C@H](C)c1cc(cc3ccccc13)CCCCCS(=O)(=O)C2] |
| 25 | 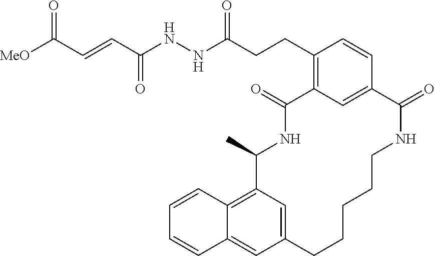 [SMILES: COC(=O)/C=C/C(=O)NNC(=O)CCc1ccc2cc1C(=O)N[C@H](C)c1cc(cc3ccccc13)CCCCCNC2=O] | 26 | 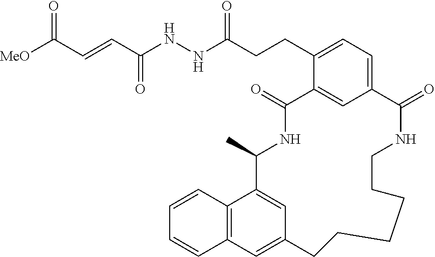 [SMILES: COC(=O)/C=C/C(=O)NNC(=O)CCc1ccc2cc1C(=O)N[C@H](C)c1cc(cc3ccccc13)CCCCCCNC2=O] |
| 27 | 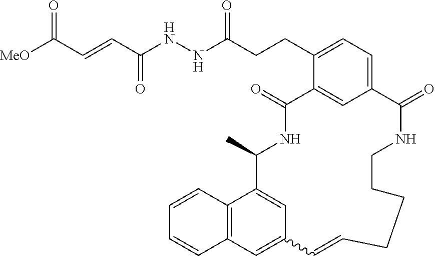 [SMILES: COC(=O)/C=C/C(=O)NNC(=O)CCc1ccc2cc1C(=O)N[C@H](C)c1cc(cc3ccccc13)C=CCCCCNC2=O] | 28 | 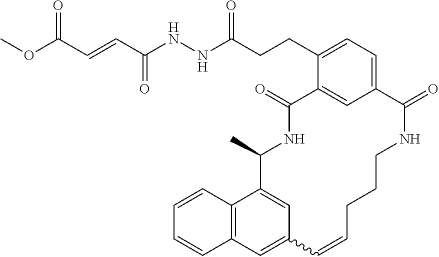 [SMILES: COC(=O)/C=C/C(=O)NNC(=O)CCc1ccc2cc1C(=O)N[C@H](C)c1cc(cc3ccccc13)C=CCCCNC2=O] |
| 29 | 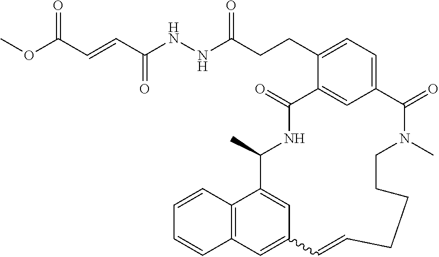 | 30 | 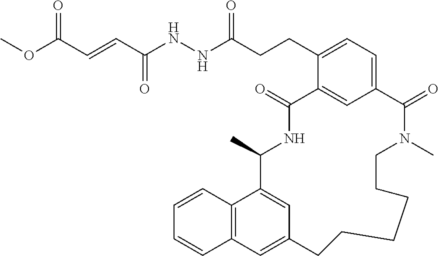 [SMILES: COC(=O)/C=C/C(=O)NNC(=O)CCc1ccc2cc1C(=O)N[C@H](C)c1cc(cc3ccccc13)CCCCCCN(C)C2=O] |
| 31 | 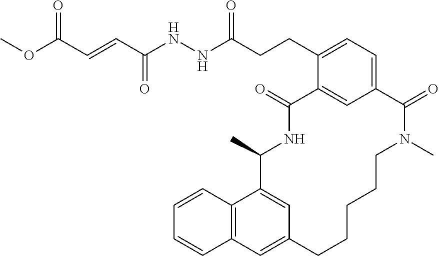 | 32 | 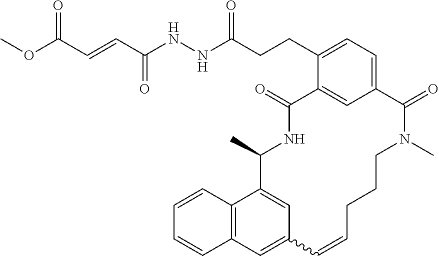 |
| 33 | 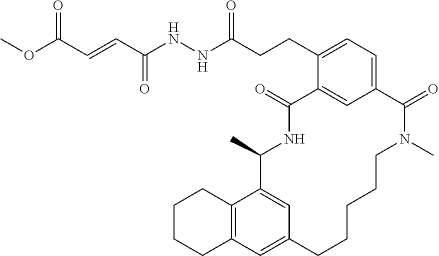 | 34 | 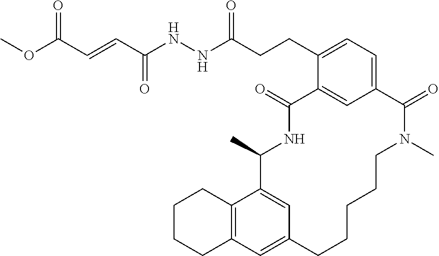 |
| 35 | 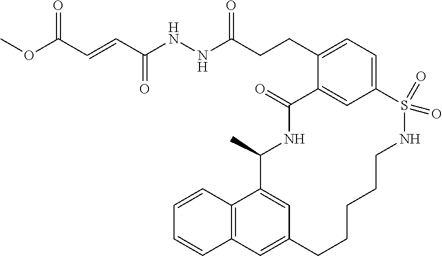 [SMILES: COC(=O)/C=C/C(=O)NNC(=O)CCc1ccc2cc1C(=O)N[C@H](C)c1cc(cc3ccccc13)CCCCCNS2(=O)=O] | 36 | 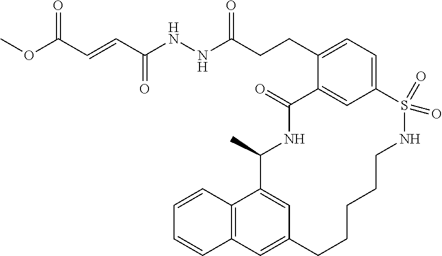 [SMILES: COC(=O)/C=C/C(=O)NNC(=O)CCc1ccc2cc1C(=O)N[C@H](C)c1cc(cc3ccccc13)CCCCCNS2(=O)=O] |
| 37 | 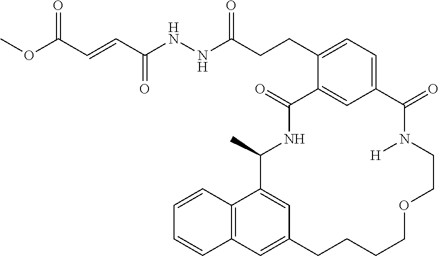 [SMILES: COC(=O)/C=C/C(=O)NNC(=O)CCc1ccc2cc1C(=O)N[C@H](C)c1cc(cc3ccccc13)CCCCOCCNC2=O] | 38 | 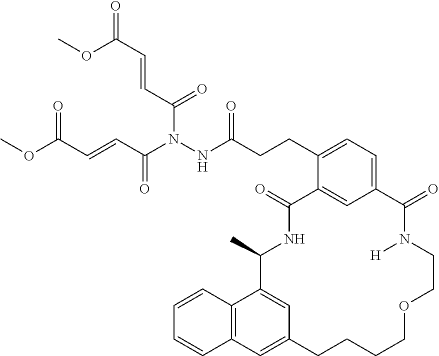 [SMILES: COC(=O)/C=C/C(=O)N(NC(=O)CCc1ccc2cc1C(=O)N[C@H](C)c1cc(cc3ccccc13)CCCCOCCNC2=O)C(=O)/C=C/C(=O)OC] |
| 39 | 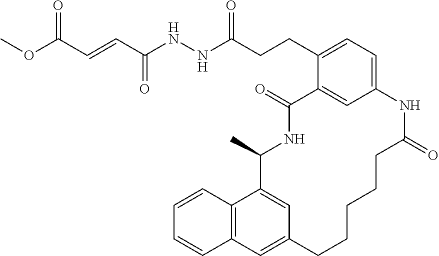 [SMILES: COC(=O)/C=C/C(=O)NNC(=O)CCc1ccc2cc1C(=O)N[C@H](C)c1cc(cc3ccccc13)CCCCCC(=O)N2] | 40 | 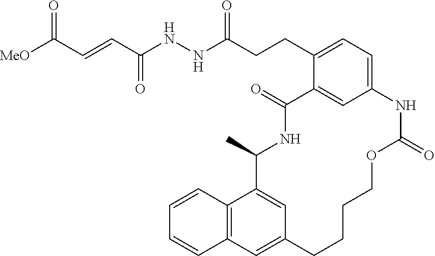 [SMILES: COC(=O)/C=C/C(=O)NNC(=O)CCc1ccc2cc1C(=O)N[C@H](C)c1cc(cc3ccccc13)CCCCOC(=O)N2] |
| 41 | 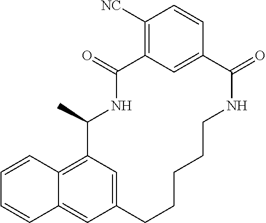 [SMILES: C[C@H]1NC(=O)c2cc(ccc2C#N)C(=O)NCCCCCc2cc1c1ccccc1c2] | 42 | 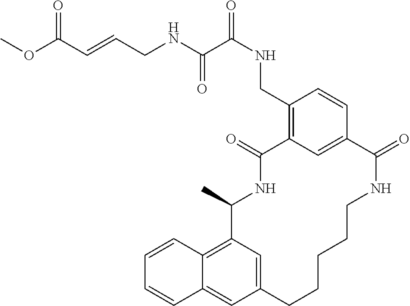 [SMILES: COC(=O)/C=C/CNC(=O)C(=O)NCc1ccc2cc1C(=O)N[C@H](C)c1cc(cc3ccccc13)CCCCCNC2=O] |

7. A pharmaceutical composition comprising a compound according to claim 1 and a pharmaceutically acceptable carrier or excipient.

8. A method of treating or preventing an infection from an RNA-based virus, a coronavirus, a rhinovirus or a norovirus, in a subject in need thereof, the method comprising administering to the subject a therapeutically effective amount of a compound according to claim 1.

9. A method of treating or preventing a coronavirus infection in a subject in need thereof, comprising administering to the subject a therapeutically effective amount of a compound or a combination of compounds according to claim 1.

10. A method of inhibiting viral Papain-Like protease in a subject, comprising administering to said subject an effective amount of a compound according to claim 1.

11. The method according to claim 10, wherein the subject is a human.

12. A method of treating a respiratory disorder selected from the group consisting of acute asthma, lung disease secondary to environmental exposures, acute lung infection, and chronic lung infection, in a subject in need thereof, comprising administering to the subject an effective amount of a compound of claim 1.

13. The method according to claim 12, wherein the compound or pharmaceutical composition is administered orally, subcutaneously, intravenously or by inhalation.
